(19) United States
(12) Patent Application Publication
(10) Pub. No.: US 2024/0371528 A1
(43) Pub. Date: Nov. 7, 2024

(54) MACHINE LEARNING FOR PREDICTING MUTATIONAL DRIVERS AND LIKELY ONSET OF FUTURE PANDEMICS

(71) Applicant: Vir Biotechnology, Inc., San Francisco, CA (US)

(72) Inventors: Michael Cyrus Riley Maher, San Mateo, CA (US); Amalio Telenti, La Jolla, CA (US)

(21) Appl. No.: 18/572,474

(22) PCT Filed: Jun. 17, 2022
(86) PCT No.: PCT/US2022/033964

§ 371 Date: Dec. 20, 2023

Related U.S. Application Data
(60) Provisional application No. 63/212,945, filed on Jun. 21, 2021.

(51) Int. Cl. G16H 50/80; G16B 20/00; G16B 40/20

(52) U.S. Cl. CPC G16H 50/80; G16B 20/00; G16B 40/20

(57) ABSTRACT

Methods disclosed herein involve forecasting mutations that will lead to pathogenic spread in the near future (e.g., 1 month, 2 months, 3 months, 4 months, or more). Using prior surveillance data including a previous spread of the pathogen, informative features of a mutation are identified for the pathogen and used to predict whether the mutation is likely to lead to future pathogenic spread. Thus, this enables early identification of future strains of prevalent pathogens which can be used to develop therapeutics (e.g., vaccines) before the spread has occurred.

### CROSS-REFERENCE TO RELATED APPLICATIONS

This application claims the benefit of and priority to U.S. Provisional Patent Application No. 63/212,945 filed Jun. 21, 2021, the entire disclosure of which is hereby incorporated by reference in its entirety for all purposes.

### BACKGROUND

Pathogenic evolution, such as influenza or SARS-COV-2 evolution, presents an ongoing challenge to public health. Many parts of the influenza and SARS-COV-2 genomes are constantly changing over time. Therefore, understanding the relative importance of mutations in viral proteins is key to allocating preparedness efforts.

In regards to SARS-COV-2, mutations in the viral Spike protein have received particular attention, because Spike is the target of antibody-mediated immunity, and is the primary antigen in current vaccines¹. As of Apr. 24, 2021, more than 6,200 distinct amino acid substitutions, insertions or deletions have been reported in Spike². These mutations occur at all but two positions in the protein, in different combinations, creating over 45,000 unique Spike protein sequences. A subset of these mutations have been classified by the Centers for Disease Control as being components of either “Variants of Interest” (VOIs) or “Variants of Concern” (VOCs). The distinction between VOIs and the higher alert VOCs is whether negative clinical impact is suspected or confirmed³.

To date, there are no efficient methods for identifying mutations in viral proteins that will lead to future pathogen spread (e.g., spread of SARS-COV-2). Given the vast number of unique protein sequences, and the continued possibility of additional mutations which further increases the number of protein sequences, it remains difficult to prepare against pathogenic spread prior to the occurrence of the spread.

### SUMMARY

Early identification of key amino acid changes contributing to future putative VOI/VOCs would be a boon to public health strategy. Such predictions could enhance the identification of liabilities for antibody-based therapeutics, vaccines and diagnostics. Predicting future mutations in variants that spread would extend the time available to develop proactive responses at earlier stages of spread. It would also complement existing forecasting efforts which seek to predict overall SARS-COV-2 incidence, hospitalizations, and death over time⁴⁻⁶. As an indication of the need for mutation-centered models, the CDC aggregates results from 25 models that predict the number of new COVID-19 cases⁷. No comparable models are in use for predicting SARS-COV-2 mutations contributing to VOI/VOCs.⁴⁻⁶

Methods disclosed herein involve forecasting mutations that will spread in the near future. This would also allow simultaneous identification of the dominant biological drivers of viral evolution over time. These two goals are mutually reinforcing: the features that are most useful for forecasting can be inferred as measuring viral fitness. Conversely, a better understanding of evolutionary dynamics can make modeling more accurate and robust. Methods involve (i) describing patterns of rapid mutation spread both globally and within the United States, (ii) elucidating the relative predictive importance of amino acid mutational features comprising immunity, transmissibility, evolution, language model, and epidemiology; (iii) utilizing data from previous waves to train and back-test a forecasting model that anticipates future spreading mutations, and (iv) illustrating how forecasted mutations could differentially affect clinical antibodies.

Evolution of pathogens, such as SARS-COV-2, threatens vaccine—and natural infection-derived immunity, and the efficacy of therapeutic antibodies. Herein Spike mutations that will occur in future variants of concern are predicted. Methods involve testing the importance of features comprising epidemiology, evolution, immunology, and neural network-based protein sequence modeling, and further involve identifying the primary biological drivers of SARS-COV-2 intra-pandemic evolution. Here, evidence was found that resistance to immune response has increasingly shaped SARS-COV-2 evolution over time. The predictive model was designed to be robust to these shifting evolutionary forces and to eliminate sources of overfitting. Using historical data sets, mutations were identified that will spread, at up to four months in advance, across different phases of the pandemic. Behavior of the model is consistent with a plausible causal structure wherein epidemiological variables integrate the effects of diverse and shifting drivers of viral fitness. The model was applied to forecast mutations that will spread in the future and characterize how these mutations could affect the binding of therapeutic antibodies. These findings demonstrate that it may be possible to forecast the mutations that will appear in future SARS-2 variants of concern. This modeling approach may be applied to any pathogen with genomic surveillance data, and so may address other mutationally diverse pathogens such as influenza, and as yet unknown future pandemic viruses.

Disclosed herein is a method for predicting spread of a mutation of a pathogen, the method comprising: obtaining features of the mutation of the pathogen; applying a predictive model to features of the mutation to predict a score indicative of a likelihood of spread of the mutation, wherein the predictive model is generated using training data derived from prior genomic, transcriptomic, or proteomic surveillance data of the pathogen corresponding to one or more previous spreads of the pathogen; and determining whether the mutation of the pathogen will spread according to the predicted score. In various embodiments, features of the mutation comprise one or more of epidemiology features, evolution features, transmissibility features, language model features, or immune features. In various embodiments, epidemiology features comprise one or more of mutation frequency, the fraction of unique variant sequences that contain an amino acid mutation, the number of countries in which a mutation was been observed, or an epidemiology score representing an exponentially weighted mean ranking across mutation frequency, the fraction of unique variant sequences that contain an amino acid mutation, and the number of countries in which a mutation was been observed. In various embodiments, language model features comprise one or more of grammaticality or semantic change scores. In various embodiments, transmissibility features comprise one or more of change in receptor binding domain (RBD) expression or ACE2 binding change. In various embodiments, immune features comprise one or more of frequency of a mutation in cytotoxic lymphocyte epitopes, percent or average CD8+ T-cell response to an epitope, percent or average CD4+ T-cell response to an epitope, an antibody binding score representing percent contribution of a site to binding of an antibody, or a maximum escape fraction for a mutation. In various embodiments, evolution features comprise one or more of positive selection features, Codon-SHAPE feature, or viral entropy features.

In various embodiments, applying the predictive model to features of the mutation comprises applying the predictive model only to epidemiology features. In various embodiments, applying the predictive model comprises applying the predictive model only to an epidemiology score. In various embodiments, the predictive model exhibits an area under the receiving operating curve (AUROC) value of at least 0.90 for predicting 1 month in advance of a forecasted spread. In various embodiments, the predictive model exhibits an area under the receiving operating curve (AUROC) value of at least 0.85 for predicting 2 months in advance of a forecasted spread. In various embodiments, the predictive model exhibits an area under the receiving operating curve (AUROC) value of at least 0.80 for predicting at least 3 months in advance of a forecasted spread. In various embodiments, the predictive model exhibits an area under the receiving operating curve (AUROC) value of at least 0.60 for predicting at least 4 months in advance of a forecasted spread. In various embodiments, the predictive model exhibits an area under the receiving operating curve (AUROC) value of at least 0.70 for predicting at least 4 months in advance of a forecasted spread. In various embodiments, the predictive model exhibits an area under the receiving operating curve (AUROC) value of at least 0.80 for predicting at least 4 months in advance of a forecasted spread. In various embodiments, the predictive model exhibits an area under the receiving operating curve (AUROC) value of at least 0.85 for predicting at least 4 months in advance of a forecasted spread.

In various embodiments, the predictive model exhibits an area under the receiving operating curve (AUROC) value of at least 0.87 for predicting at least 4 months in advance of a forecasted spread. In various embodiments, the mutation is an amino acid mutation of a protein of the pathogen. In various embodiments, the mutation is a nucleic acid mutation corresponding to an amino acid change of a protein of the pathogen. In various embodiments, methods disclosed herein further comprise predicting impact of the mutation on therapeutic efficacy of therapeutic antibody. In various embodiments, predicting impact of the mutation comprises: mapping the mutation to a specific amino acid of a protein of the pathogen; and determining a contribution of the mutation of the specific amino acid to a binding energy between the therapeutic antibody and the protein of the pathogen.

In various embodiments, methods disclosed herein further comprise: subsequent to determining that the mutation of the pathogen will spread according to the predicted score, identifying a pathogen variant likely to spread, the pathogen variant comprising at least the determined mutation that will spread. In various embodiments, the pathogen variant further comprises at least two, at least three, at least four, at least five, at least six, at least seven, at least eight, at least nine, at least ten, at least eleven, at least twelve, at least thirteen, at least fourteen, at least fifteen at least sixteen, at least seventeen, at least eighteen, at least nineteen, at least twenty, at least twenty five, at least thirty, at least thirty five, at least forty, at least forty five, at least fifty, at least fifty five, at least sixty, at least sixty five, at least seventy, at least seventy five, at least eighty, at least eighty five, at least ninety, at least ninety five, or at least a hundred additional mutations that are predicted to likely spread. In various embodiments, the identification of the pathogen variant likely to spread is based on one or more prior variants of interest or variants of concern. In various embodiments, the identification of the pathogen variant likely to spread is based on one or more prior variants of interest or variants of concern and additional one or more mutations that occur at a rate of at least a threshold percentage of a most prevalent variant in the lineage. In various embodiments, the threshold percentage is at least 50%, at least 55%, at least 60%, at least 65%, at least 70%, at least 75%, at least 80%, at least 85%, at least 90%, or at least 95%.

Additionally disclosed herein is a method for training a predictive model capable of forecasting one or more spreading mutations of a pathogen, the method comprising: obtaining one or more of prior surveillance data of the pathogen; defining spread of one or more mutations in the prior surveillance data of the pathogen; performing a feature selection process to identify one or more features informative for predicting spread of the defined one or more mutations; and training a predictive model using training data comprising values of the identified one or more features, the training data derived from the surveillance data of the pathogen. In various embodiments, defining spread of one or more mutations comprises: for a mutation, determining one or more fold changes in frequency of the mutation within a time window in comparison to a previous time window; and comparing the determined one or more fold changes to a threshold fold-change value.

In various embodiments, each of the one or more fold changes in frequency of the mutation is calculated for a country. In various embodiments, each of the one or more fold changes in frequency of the mutation is calculated for a state. In various embodiments, defining spread of one or more mutations comprises: determining spread of a first mutation of the pathogen corresponding to a first wave; and determining spread of a second mutation of the pathogen corresponding to a second wave. In various embodiments, the first wave and the second wave occur within 1 year. In various embodiments, the first wave and the second wave are separated by at least 1 year.

In various embodiments, wherein the one or more features informative for predicting spread of the defined one or more mutations comprise one or more of epidemiology features, evolution features, transmissibility features, language model features, or immune features. In various embodiments, epidemiology features comprise one or more of mutation frequency, the fraction of unique variant sequences that contain an amino acid mutation, the number of countries in which a mutation was been observed, or an epidemiology score representing an exponentially weighted mean ranking across mutation frequency, the fraction of unique variant sequences that contain an amino acid mutation, and the number of countries in which a mutation was been observed. In various embodiments, language model features comprise one or more of grammaticality or semantic change scores. In various embodiments, transmissibility features comprise one or more of change in receptor binding domain (RBD) expression or ACE2 binding change. In various embodiments, immune features comprise one or more of frequency of a mutation in cytotoxic lymphocyte epitopes, percent or average CD8+ T-cell response to an epitope, percent or average CD4+ T-cell response to an epitope, an antibody binding score representing percent contribution of a site to binding of an antibody, or a maximum escape fraction for a mutation. In various embodiments, evolution features comprise one or more of positive selection features, Codon-SHAPE feature, or viral entropy features. In various embodiments, the pathogen is an epidemic or pandemic causing pathogen. In various embodiments, the pathogen is either influenza or SARS-COV-2. In various embodiments, the surveillance data comprises one or more of genomic, transcriptomic, or proteomic surveillance data.

Additionally disclosed herein is a non-transitory computer readable medium for predicting spread of a mutation of a pathogen, the non-transitory computer readable medium comprising instructions that, when executed by a processor, cause the processor to: obtain features of the mutation of the pathogen; apply a predictive model to features of the mutation to predict a score indicative of a likelihood of spread of the mutation, wherein the predictive model is generated using training data derived from prior surveillance data of the pathogen corresponding to one or more previous spreads of the pathogen; and determine whether the mutation of the pathogen will spread according to the predicted score. In various embodiments, features of the mutation comprise one or more of epidemiology features, evolution features, transmissibility features, language model features, or immune features. In various embodiments, epidemiology features comprise one or more of mutation frequency, the fraction of unique variant sequences that contain an amino acid mutation, the number of countries in which a mutation was been observed, or an epidemiology score representing an exponentially weighted mean ranking across mutation frequency, the fraction of unique variant sequences that contain an amino acid mutation, and the number of countries in which a mutation was been observed. In various embodiments, language model features comprise one or more of grammaticality or semantic change scores. In various embodiments, transmissibility features comprise one or more of change in receptor binding domain (RBD) expression or ACE2 binding change. In various embodiments, immune features comprise one or more of frequency of a mutation in cytotoxic lymphocyte epitopes, percent or average CD8+ T-cell response to an epitope, percent or average CD4+ T-cell response to an epitope, an antibody binding score representing percent contribution of a site to binding of an antibody, or a maximum escape fraction for a mutation. In various embodiments, evolution features comprise one or more of positive selection features, Codon-SHAPE feature, or viral entropy features. In various embodiments, the instructions that cause the processor to apply the predictive model to features of the mutation further comprises instructions that, when executed by the processor, cause the processor to apply the predictive model only to epidemiology features. In various embodiments, the instructions that cause the processor to apply the predictive model comprises instructions that, when executed by the processor, cause the processor to apply the predictive model only to an epidemiology score.

In various embodiments, the predictive model exhibits an area under the receiving operating curve (AUROC) value of at least 0.90 for predicting 1 month in advance of a forecasted spread. In various embodiments, the predictive model exhibits an area under the receiving operating curve (AUROC) value of at least 0.85 for predicting 2 months in advance of a forecasted spread. In various embodiments, the predictive model exhibits an area under the receiving operating curve (AUROC) value of at least 0.80 for predicting at least 3 months in advance of a forecasted spread. In various embodiments, the predictive model exhibits an area under the receiving operating curve (AUROC) value of at least 0.60 for predicting at least 4 months in advance of a forecasted spread.

In various embodiments, the predictive model exhibits an area under the receiving operating curve (AUROC) value of at least 0.70 for predicting at least 4 months in advance of a forecasted spread. In various embodiments, the predictive model exhibits an area under the receiving operating curve (AUROC) value of at least 0.80 for predicting at least 4 months in advance of a forecasted spread. In various embodiments, the predictive model exhibits an area under the receiving operating curve

(AUROC) value of at least 0.85 for predicting at least 4 months in advance of a forecasted spread. In various embodiments, the predictive model exhibits an area under the receiving operating curve (AUROC) value of at least 0.87 for predicting at least 4 months in advance of a forecasted spread.

In various embodiments, the mutation is an amino acid mutation of a protein of the pathogen. In various embodiments, the mutation is a nucleic acid mutation corresponding to an amino acid change of a protein of the pathogen. In various embodiments, the non-transitory computer readable medium further comprises instructions that, when executed by the processor, cause the processor to predict impact of the mutation on therapeutic efficacy of therapeutic antibody. In various embodiments, the instructions that cause the processor to predict impact of the mutation further comprises instructions that, when executed by the processor, cause the processor to: map the mutation to a specific amino acid of a protein of the pathogen; and determine a contribution of the mutation of the specific amino acid to a binding energy between the therapeutic antibody and the protein of the pathogen.

In various embodiments, the non-transitory computer readable medium disclosed herein, further comprises instructions that, when executed by the processor, cause the processor to: subsequent to the determination that the mutation of the pathogen will spread according to the predicted score, identify a pathogen variant likely to spread, the pathogen variant comprising at least the determined mutation that will spread. In various embodiments, the pathogen variant further comprises at least two, at least three, at least four, at least five, at least six, at least seven, at least eight, at least nine, at least ten, at least eleven, at least twelve, at least thirteen, at least fourteen, at least fifteen at least sixteen, at least seventeen, at least eighteen, at least nineteen, at least twenty, at least twenty five, at least thirty, at least thirty five, at least forty, at least forty five, at least fifty, at least fifty five, at least sixty, at least sixty five, at least seventy, at least seventy five, at least eighty, at least eighty five, at least ninety, at least ninety five, or at least a hundred additional mutations that are predicted to likely spread. In various embodiments, the identification of the pathogen variant likely to spread is based on one or more prior variants of interest or variants of concern. In various embodiments, the identification of the pathogen variant likely to spread is based on one or more prior variants of interest or variants of concern and additional one or more mutations that occur at a rate of at least a threshold percentage of a most prevalent variant in the lineage. In various embodiments, the threshold percentage is at least 50%, at least 55%, at least 60%, at least 65%, at least 70%, at least 75%, at least 80%, at least 85%, at least 90%, or at least 95%.

Additionally disclosed herein is a non-transitory computer readable medium for training a predictive model capable of forecasting one or more spreading mutations of a pathogen, the non-transitory computer readable medium comprising instructions that, when executed by a processor, cause the processor to: obtain one or more of prior surveillance data of the pathogen; define spread of one or more mutations in the prior surveillance data of the pathogen; perform a feature selection process to identify one or more features informative for predicting spread of the defined one or more mutations; and train a predictive model using training data comprising values of the identified one or more features, the training data derived from the surveillance data of the pathogen. In various embodiments, the instructions that cause the processor to define spread of one or more mutations further comprises instructions that, when executed by the processor, cause the processor to: for a mutation, determine one or more fold changes in frequency of the mutation within a time window in comparison to a previous time window; and compare the determined one or more fold changes to a threshold fold-change value.

In various embodiments, each of the one or more fold changes in frequency of the mutation is calculated for a country. In various embodiments, each of the one or more fold changes in frequency of the mutation is calculated for a state. In various embodiments, the instructions that cause the processor to define spread of one or more mutations further comprises instructions that, when executed by the processor, cause the processor to: determine spread of a first mutation of the pathogen corresponding to a first wave; and determine spread of a second mutation of the pathogen corresponding to a second wave. In various embodiments, the first wave and the second wave occur within 1 year. In various embodiments, the first wave and the second wave are separated by at least 1 year.

In various embodiments, the one or more features informative for predicting spread of the defined one or more mutations comprise one or more of epidemiology features, evolution features, transmissibility features, language model features, or immune features. In various embodiments, epidemiology features comprise one or more of mutation frequency, the fraction of unique variant sequences that contain an amino acid mutation, the number of countries in which a mutation was been observed, or an epidemiology score representing an exponentially weighted mean ranking across mutation frequency, the fraction of unique variant sequences that contain an amino acid mutation, and the number of countries in which a mutation was been observed. In various embodiments, language model features comprise one or more of grammaticality or semantic change scores. In various embodiments, transmissibility features comprise one or more of change in receptor binding domain (RBD) expression or ACE2 binding change. In various embodiments, immune features comprise one or more of frequency of a mutation in cytotoxic lymphocyte epitopes, percent or average CD8+ T-cell response to an epitope, percent or average CD4+ T-cell response to an epitope, an antibody binding score representing percent contribution of a site to binding of an antibody, or a maximum escape fraction for a mutation. In various embodiments, evolution features comprise one or more of positive selection features, Codon-SHAPE feature, or viral entropy features. In various embodiments, the pathogen is an epidemic or pandemic causing pathogen. In various embodiments, the pathogen is either influenza or SARS-COV-2. In various embodiments, the surveillance data comprises one or more of genomic, transcriptomic, or proteomic surveillance data.

Additionally disclosed herein is a method for identifying a pathogen variant likely to spread, the method comprising: obtaining values of features of one or more mutations of a pathogen; for one of the one or more mutations: applying a predictive model to values of features of the mutation to predict a score indicative of a likelihood of spread of the mutation, wherein the predictive model is generated using training data derived from prior surveillance data of the pathogen corresponding to one or more previous spreads of the pathogen; and determining that the mutation will spread according to the predicted score; and identifying a pathogen variant likely to spread, the pathogen variant comprising at least the determined mutation that will spread. In various embodiments, the pathogen variant further comprises at least two, at least three, at least four, at least five, at least six, at least seven, at least eight, at least nine, at least ten, at least eleven, at least twelve, at least thirteen, at least fourteen, at least fifteen at least sixteen, at least seventeen, at least eighteen, at least nineteen, at least twenty, at least twenty five, at least thirty, at least thirty five, at least forty, at least forty five, at least fifty, at least fifty five, at least sixty, at least sixty five, at least seventy, at least seventy five, at least eighty, at least eighty five, at least ninety, at least ninety five, or at least a hundred additional mutations that are predicted to likely spread. In various embodiments, the identification of the pathogen variant likely to spread is based on one or more prior variants of interest or variants of concern. In various embodiments, the identification of the pathogen variant likely to spread is based on one or more prior variants of interest or variants of concern and additional one or more mutations that occur at a rate of at least a threshold percentage of a most prevalent variant in the lineage. In various embodiments, the threshold percentage is at least 50%, at least 55%, at least 60%, at least 65%, at least 70%, at least 75%, at least 80%, at least 85%, at least 90%, or at least 95%.

### BRIEF DESCRIPTION OF THE DRAWINGS

These and other features, aspects, and advantages of the present invention will become better understood with regard to the following description, and accompanying drawings, where:

FIG. 1A depicts a block diagram of a pathogen prediction system, in accordance with an embodiment.

FIG. 1B shows an example schematic for analyzing various windows of surveillance data to predict whether mutations are likely to spread in the future, in accordance with an embodiment.

FIG. 2A is an example flow process for predicting whether a mutation of a pathogen will spread, in accordance with an embodiment.

FIG. 2B is an example flow process for training a predictive model for use in predicting future spread of mutations.

FIG. 3 illustrates an example computing device 300 for implementing methods and systems described in FIGS. 1, 2A, and 2B.

FIGS. 4A-4C depict an example baseline study design using sliding windows to predict spread of pathogens.

FIGS. 5A and 5B describe spreading mutations in the third SARS-COV-2 wave.

FIG. 6A shows the most predictive variables within each feature group for the receptor binding domain (RBD) and Spike protein.

FIG. 6B shows RBD classification accuracy over time for the top GISAID-based feature (Epi score), and the top transmission and immune variables.

FIG. 6C shows performance for identifying which mutations will spread during the baseline analysis period.

FIGS. 7A-7D show the early detection and causal mediation of spreading mutations.

FIG. 8 shows all non-canonical variants highlighted for third wave of the SARS-COV-2 analysis.

FIGS. 9A-9C show changes in predictiveness of various features over time. In particular, AUROC for all variables, over 10 sliding window periods are shown for RBD (FIG. 9A) and Spike (FIG. 9B).

FIG. 10 shows the predictive performance of integrated feature sets at baseline.

FIG. 11 shows graphs predicting local and global spreading mutations in two separate waves.

FIG. 12 shows graphs of the performance as a function of number of variants.

FIG. 13 is a depiction of growth trajectories of various mutations and when they were first forecast to spread.

FIG. 14 shows the effect of the length of the feature calculation window on predictive performance.

FIG. 15 shows population size-adjusted mutation frequencies for mutations forecasted to spread.

FIG. 16 shows the relationship between predicted probability of spread and actual prevalence of certain mutations. For example, a number of Omicron variants (e.g., BA.2, BA.2.12, BA.2.12.1, BA.4, BA.5) were predicted to spread, which matches with their actual high prevalence.

FIG. 17 shows predictive performance of the model over 18 sliding window periods.

It must be noted that, as used in the specification and the appended claims, the singular forms “a,” “an” and “the” include plural referents unless the context clearly dictates otherwise.

### DETAILED DESCRIPTION OF THE INVENTION

### Overview

Generally, methods disclosed herein are useful for predicting whether particular pathogenic mutations will likely lead to future spread of pathogen variants including the one or more of the mutations. By identifying pathogenic mutations that are likely to spread prior to their actual spread, new therapeutic modalities can be developed and rapidly deployed to combat the identified pathogen variants that harbor one or more of the identified mutations. Given the significant toll that recent pandemics, such as the SARS-COV-2 pandemic, has had on society, methods disclosed herein present promising strategies for effective curtailing and shortening the duration of pathogenic spread.

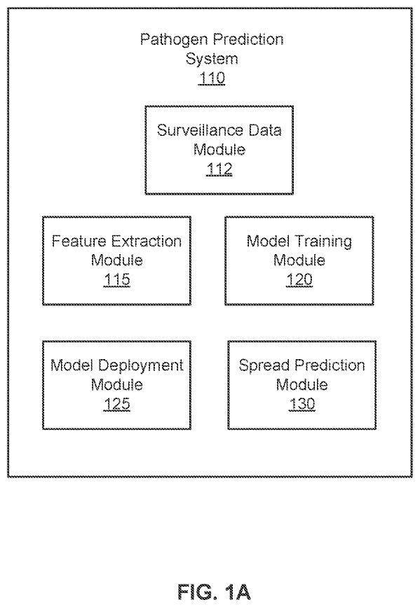

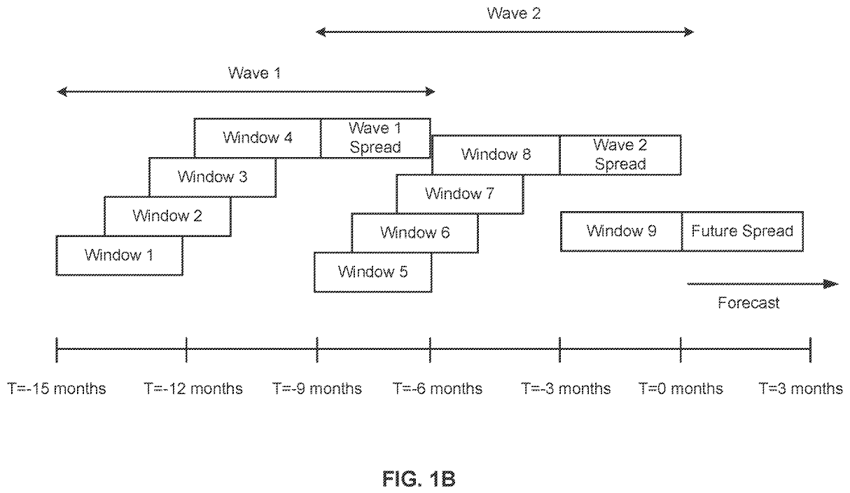

Figure (FIG. 1A depicts a block diagram of a pathogen prediction system 110, in accordance with an embodiment. The pathogen prediction system 110 can perform the methods described herein for predicting the likely spread of pathogens with one or more pathogenic mutations. As used herein, a “mutation” includes any of polymorphisms, single nucleotide polymorphisms (SNPs), single nucleotide variants (SNVs)), insertions, deletions, knock-ins, knock-outs, copy number variations (CNVs), duplications, translocations, and loss of heterozygosity (LOH). In particular embodiments, a “mutation” refers to a single nucleotide variant (SNV). Such pathogenic mutations may cause a change in the pathogen, such as increasing or decreasing the transmissibility of the pathogen and/or increasing or decreasing the severity of an infection caused by the pathogen.

The pathogen prediction system 110 is shown here to introduce the modules of the pathogen prediction system 110, which includes, in various embodiments, a surveillance data module 112, a feature extraction module 115, a model training module 120, a model deployment module 125, and a spread prediction module 130. In various embodiments, the pathogen prediction system 110 may be differently configured than as shown in FIG. 1A. For example, the model training module 120 may be implemented by a different party/system and need not be included in the pathogen prediction system 110. For example, in a scenario where the training and deployment of predictive models are performed by different parties, the model training module 120, which implements methods for training a predictive model, can be implemented by a first party and the model deployment module 125 may be a part of the pathogen prediction system 110 that is operated by a second party.

Generally, the surveillance data module 112 obtains and processes the surveillance data. In various embodiments, the surveillance data module 112 divides up the surveillance data into time windows, such that individual time windows of the surveillance data are more manageable and can be separately analyzed. This enables the subsequent training of predictive models and deployment of predictive models on particular time windows. The feature extraction module 115 analyzes surveillance data (e.g., genomic, transcriptomic, or proteomic surveillance data) and extracts values of features from the surveillance data. The model training module 120 trains one or more predictive models using training data generated e.g., by the feature extraction module 115 from surveillance data. For example, in various embodiments, the feature extraction module 115 performs a feature extraction to generate training data that can be used by the model training module 120 for training a predictive model. The model deployment module 125 deploys a trained predictive model to predict whether a mutation is likely to spread in the future. For example, in various embodiments, the feature extraction module 115 performs a feature extraction such that the model deployment module 125 can apply a trained predictive model to analyze the extracted feature values to predict a probability that a mutation is likely to spread. The spread prediction module 130 analyzes outputs of the predictive models and determines which pathogen (e.g., a pathogen variant) is likely to spread in the future. In various embodiments, a pathogen that is likely to spread in the future are haplotypes of concern and therefore, can be deemed a variant of interest (VOI) or a variant of concern (VOC).

In various embodiments, the spread prediction module 130 determines that a particular mutation will lead to likely spread of a pathogen variant harboring the particular mutation. In various embodiments, the spread prediction module 130 determines that one or more mutations will lead to likely spread of a pathogen variant harboring the one or more mutations. For example, the spread prediction module 130 can determine that a particular combination of mutations is highly likely to spread, and therefore, a pathogen variant that harbors this combination of mutations is likely to spread in the future. Further details of the steps performed by the surveillance data module 112, feature extraction module 115, the model training module 120, the model deployment module 125, and the spread prediction module 130 are described herein.

Methods for Identifying Mutations and/or Pathogen Variants that are Likely to Spread

Disclosed herein are methods for identifying mutations and/or pathogen variants that are likely to spread in the future. Methods for identifying mutations and/or pathogen variants that are likely to spread in the future can involve two separate phases: 1) a training phase and 2) a deployment phase. In various embodiments, during the training phase, methods involve obtaining prior surveillance data of the pathogen, defining one or more prior spreads of the pathogen in the prior surveillance data, and identifying features that are informative for predicting the prior spread of the pathogen. Methods further involve training a predictive model using the features that are informative for predicting the prior spread of the pathogen. In various embodiments, during the deployment phase, methods involve extracting values of features for a mutation from surveillance data and then deploying a trained predictive model to predict whether the mutation is likely to spread in the future. Pathogen variants harboring one or more mutations that are likely to spread in the future can be identified as likely to spread.

### Analyzing Surveillance Data

FIG. 1B shows an example schematic for analyzing various time windows of prior surveillance data to predict whether mutations are likely to spread in the future. In various embodiments, the surveillance data can include any of genomic surveillance data, transcriptomic surveillance data, or proteomic surveillance data of the pathogen. In particular embodiments, the surveillance data includes genomic surveillance data. In various embodiments, the surveillance data module 112 (shown in FIG. 1A) divides up the surveillance data into separate time windows (e.g., shown in FIG. 1B as Window 1, Window 2 . . . . Window 8). In some embodiments, the surveillance data module 112 divides up the surveillance data into additional or fewer time windows in comparison to the 8 windows shown in FIG. 1B. In various embodiments, the surveillance data module 112 divides up the surveillance data into at least two, at least three, at least four, at least five, at least six, at least seven, at least eight, at least nine, at least ten, at least eleven, at least twelve, at least thirteen, at least fourteen, at least fifteen at least sixteen, at least seventeen, at least eighteen, at least nineteen, at least twenty, at least twenty five, at least thirty, at least thirty five, at least forty, at least forty five, at least fifty, at least fifty five, at least sixty, at least sixty five, at least seventy, at least seventy five, at least eighty, at least eighty five, at least ninety, at least ninety five, or at least a hundred time windows.

In various embodiments, the surveillance data module 112 divides up the surveillance data into equal time windows. For example, each time window may correspond to a time period of 1 month. As another example, each time window may correspond to a time period of 2 months. As yet another example, each time window may correspond to a time period of 3 months. In various embodiments, each time window may correspond to a time period of at least 1 day, at least 2 days, at least 3 days, at least 4 days, at least 5 days, at least 6 days, at least 1 week, at least 2 weeks, at least 3 weeks, at least 4 weeks, at least 5 weeks, at least 6 weeks, at least 7 weeks, at least 8 weeks, at least 9 weeks, or at least 10 weeks. As shown in FIG. 1B, a time window corresponds to a time period of 3 months. In various embodiments, the surveillance data module 112 divides up the surveillance data into non-equal time windows. For example, a first time window may correspond to 1 month whereas a second time window may correspond to 2 months.

In various embodiments, the surveillance data module 112 analyzes the surveillance data and defines one or more prior spreads of one or more mutations of a pathogen. In particular, FIG. 1B shows that a particular pathogen may have had two prior waves (termed “Wave 1” and “Wave 2”) at two different time intervals in the prior surveillance data. Specifically, Wave 1 occurred between 6 and 15 months ago (e.g., identified as T=−15 months and T=−6 months) whereas Wave 2 occurred between 0 months and 9 months ago (e.g., identified as T=−9 months and T=0 months). In various embodiments, the prior surveillance data can include fewer or additional prior waves in which the pathogen may have spread. Here, Wave 1 includes a mutation that spread between T=−9 months and T=−6 months (labeled as Wave 1 Spread). Wave 2 includes a mutation that spread between T=−3 months and T=0 months (labeled as Wave 2 spread). Thus, the surveillance data module 112 analyzes the surveillance data and defines the Wave 1 spread and/or the Wave 2 spread according to embodiments of the method described below.

In various embodiments, the surveillance data module 112 defines a spread of a mutation according to a frequency of the mutation present in a geographical location. In various embodiments, a geographical location may be worldwide and thus, the surveillance data module 112 defines spread according to a frequency of the mutation across the world. In various embodiments, a geographical location may be a continent (e.g., Asia, Europe, North America, South America, Africa, etc.). In various embodiments, a geographical location may be a country (e.g., United

States, Germany, France, United Kingdom, China, etc.). Thus, in such embodiments, the surveillance data module 112 defines a country-specific spread. In various embodiments, a geographical location may be a state (e.g., one of the 50 states in the USA). Thus, in such embodiments, the surveillance data module 112 defines state-specific spread.

In various embodiments, the surveillance data module 112 defines whether a mutation has spread /according to an absolute frequency of a mutation in a geographical location. For example, if the absolute frequency of a mutation is above a threshold frequency, then the surveillance data module 112 defines the mutation as having spread. Alternatively, if the absolute frequency of a mutation is below a threshold frequency, then the surveillance data module 112 does not define the mutation as having spread. In various embodiments, the threshold frequency is a frequency of 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1.0%. In particular embodiments, the surveillance data module 112 defines the mutation as having spread if the absolute frequency of the mutation across the world is above a threshold frequency of 0.1%

In various embodiments, the surveillance data module 112 defines whether a mutation has according to a fold change of the frequency of the mutation present in one or more geographical locations. For example, to determine a fold change of the frequency of the mutation, the surveillance data module 112 may determine the frequency of the mutation at a first time window and a second time window. The first time window and second time window may be consecutive time windows and therefore, the fold change represents the change in frequency of the mutation across consecutive time windows. In various embodiments, the surveillance data module 112 defines spread of a mutation if at least one country experiences at least a 5-fold, at least a 10-fold, at least a 15-fold, or at least a 20-fold change in the mutation frequency. In particular embodiments, the surveillance data module 112 defines spread of a mutation if at least one country experiences at least a 10-fold change in the mutation frequency. In various embodiments, the surveillance data module 112 defines spread of a mutation if at least 2 countries experience at least a 2-fold, 3-fold, 4-fold, or 5-fold change in the mutation frequency. In various embodiments, the surveillance data module 112 defines spread of a mutation if at least 3 countries experience at least a 2-fold, 3-fold, 4-fold, or 5-fold change in the mutation frequency. In particular embodiments, the surveillance data module 112 defines spread of a mutation if at least 3 countries experience at least a 2-fold change in the mutation frequency.

Once the surveillance data module 112 has defined one or more prior spreads (e.g., Wave 1 spread and/or Wave 2 spread as shown in FIG. 1B), the feature extraction module 115 can perform an analysis of features of the surveillance data to identify features that are informative for predictive the prior spreads. In various embodiments, the feature extraction module 115 analyzes surveillance data of one or more windows preceding the prior spread. For example, referring to FIG. 1B, for the Wave 1 spread which occurs between T=−9 months and T=−6 months, the feature extraction module 115 may analyze the time windows preceding the Wave 1 spread, including one or more of Window 1, Window 2, Window 3, and/or Window 4. Here, the feature extraction module 115 extracts values of features and can perform feature selection to identify the features that are informative for predicting the Wave 1 Spread.

In various embodiments, the feature extraction module 115 analyzes surveillance data of multiple windows preceding the prior spread and identifies features from the multiple windows that are informative for predicting the prior spread. For example, referring to FIG. 1B, the feature extraction module 115 analyzes surveillance data from two or more, three or more, or each of of Window 1, Window 2, Window 3, and Window 4 and identifies features that are most informative for predicting the Wave 1 spread. Thus, these most informative features can be selected for inclusion in the predictive model.

In various embodiments, the feature extraction module 115 can perform separate feature selections using the different preceding time windows. For example, referring again to FIG. 1B, the feature extraction module 115 can extract values of features from Window 1 to identify features that are informative for predicting the Wave 1 Spread. Here, given that Window 1 occurs between 3-6 months prior to Wave 1 spread, the identified features from Window 1 can be informative for predicting spread of a mutation approximately 3-6 months before the mutation actually spreads. As another example, the feature extraction module 115 can extract values of features from Window 2 to identify features that are informative for predicting the Wave 1 Spread. Here, given that Window 2 occurs between 2-5 months prior to Wave 1 spread, the identified features from Window 1 can be informative for predicting spread of a mutation approximately 2-5 months before the mutation actually spreads. Similarly, the features identified from Window 3 can be informative for predicting spread of a mutation approximately 1-4 months before actual spread, and the features identified from Window 4 can be informative for predicting spread of a mutation approximately 0-3 months before actual spread.

As shown in FIG. 1B, there is also a more recent Wave 2 spread. Thus, the feature extraction module 115 can similarly extract features and perform a feature selection to identify features from Windows 5, 6, 7, and/or 8 that are informative for predicting the Wave 2 spread.

In various embodiments, the feature extraction module 115 may select the features that are most informative for predicting a prior spread (e.g., Wave 1 Spread or Wave 2 Spread). In various embodiments, the feature extraction module 115 selects the features that are most informative for predicting multiple prior spreads (e.g., features that are informative for predicting both Wave 1 Spread and Wave 2 spread). Thus, the features selected by the feature extraction module 115 can be included in a model for training the model to accurately predict whether a mutation is likely to spread in the future.

### Training a Predictive Model

Embodiments disclosed herein describe the training of predictive models for predicting mutations and/or pathogen variants that are likely to spread in the future. Referring to FIG. 1A, the model training module 120 performs the steps to train the predictive model. In various embodiments, the predictive model is any one of a regression model (e.g., linear regression, logistic regression, or polynomial regression), decision tree, random forest, support vector machine, Naïve Bayes model, k-means cluster, or neural network (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks). In particular embodiments, the predictive model is a logistic regression model. In particular embodiments, the predictive model is a random forest model. The predictive model can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, and gradient boosting algorithm.

In various embodiments, the predictive model has one or more parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, support vectors in a support vector machine, and coefficients in a regression model. The model parameters of the machine learning model are trained (e.g., adjusted) using the training data to improve the predictive power of the predictive model.

In various embodiments, the predictive model is structured to receive, as input, values for one or more features. As described herein, example features may be categorized in any one of epidemiology features, evolution features, transmissibility features, language model features, or immune features. In various embodiments, the predictive model is structured to receive, as input, values of features comprising epidemiology features. In various embodiments, the predictive model is structured to receive, as input, values of features comprising evolution features. In various embodiments, the predictive model is structured to receive, as input, values of features comprising immune features. In various embodiments, the predictive model is structured to receive, as input, values of features comprising transmissibility features. In various embodiments, the predictive model is structured to receive, as input, values of features comprising evolution, immune, and transmissibility features. In various embodiments, the predictive model is structured to receive, as input, values of features including only epidemiology features. In various embodiments, the predictive model is structure to receive, as input, a value of an Epi Score. Generally, the predictive model analyzes the values of one or more features and predicts a probability that the mutation is likely to spread within a time period in the future (e.g., within 1 month, within 2 months, within 3 months, within 4 months, within 5 months, within 6 months, within 7 months, within 8 months, within 9 months, within 10 months, within 11 months, or within 12 months).

In various embodiments, the model training module 120 provides ground truth data to train the model. For example, given that the surveillance data module 112 had previously defined prior spreads (e.g., Wave 1 Spread and/or Wave 2 Spread), the ground truth data can reflect that a prior spread had occurred. In various embodiments, the ground truth data can be a binary value (e.g., “0” or “1”). For example, a ground truth value of 1 can be indicative of a prior spread whereas a ground truth value of 0 can be indicative of the lack of a prior spread. In various embodiments, the ground truth value may be a continuous value, such as a continuous value between 0 and 1, where a value closer to 1 is indicative of a prior spread whereas a value closer to 0 is indicative of the lack of a prior spread.

In various embodiments, the model training module 120 provides, as input to the predictive model, the values of selected features of mutations that were extracted by the feature extraction module 115. The predictive model analyzes the values of the features and predicts whether the mutation is likely to spread. The prediction from the predictive model is compared to the ground truth and the parameters of the predictive model are adjusted to increase the predictive capacity of the predictive model. For example, if the predictive model predicted that a mutation is unlikely to spread when in fact, the mutation did spread, the parameters of the predictive model are adjusted to improve the subsequent predictive accuracy of the model.

### Deploying a Predictive Model to Predict Future Spread

Referring again to FIG. 1A, the model deployment module 125 deploys a trained predictive model to predict whether a mutation is likely to spread. Here, the model deployment module 125 retrieves a predictive model that was trained using surveillance data including one or more prior spreads. Thus, the predictive model can predict whether a mutation of the pathogen can likely lead to future spread. Referring to FIG. 1B, the predictive model may have been trained using surveillance data of the prior spreads. For example, the predictive model may have been trained using surveillance data from windows 1, 2, 3, and/or 4 e.g., to predict likely spread of mutations in the Wave 1 Spread. As another example, the predictive model may have been trained using surveillance data from windows 5, 6, 7, and/or 8 e.g., to predict likely spread of mutations in the Wave 2 Spread. As another example, the predictive model may have been trained using surveillance data from windows 1, 2, 3, 4, 5, 6, 7, and/or 8 e.g., to predict likely spread of mutations in the Wave 1 and Wave 2 Spread.

The model deployment module 125 deploys the trained predictive model to forecast the future likely spread of the mutation. For example, as shown in FIG. 1B, the model deployment module 125 may deploy the trained predictive model to predict future spread, which occurs between T=0 and T=3 months. Although FIG. 1B shows a prediction of future spread of a mutation between 0 and 3 months, in some embodiments, the prediction may be for future spread of a mutation at different time points in the future. In various embodiments, the predictive model predicts the likelihood of spread at least 1 month in advance of a forecasted spread. In various embodiments, the predictive model predicts the likelihood of spread at least 2 months in advance of a forecasted spread. In various embodiments, the predictive model predicts the likelihood of spread at least 3 months in advance of a forecasted spread. In various embodiments, the predictive model predicts the likelihood of spread at least 4 months in advance of a forecasted spread. In various embodiments, the predictive model predicts the likelihood of spread at least 5 months in advance of a forecasted spread. In various embodiments, the predictive model predicts the likelihood of spread at least 6 months in advance of a forecasted spread. In various embodiments, the predictive model predicts the likelihood of spread at least 7 months in advance of a forecasted spread. In various embodiments, the predictive model predicts the likelihood of spread at least 8 months in advance of a forecasted spread. In various embodiments, the predictive model predicts the likelihood of spread at least 9 months in advance of a forecasted spread. In various embodiments, the predictive model predicts the likelihood of spread at least 10 months in advance of a forecasted spread. In various embodiments, the predictive model predicts the likelihood of spread at least 11 months in advance of a forecasted spread. In various embodiments, the predictive model predicts the likelihood of spread at least 12 months in advance of a forecasted spread.

In various embodiments, the predictive model predicts the likelihood of spread at least 5 days, at least 8 days, at least 10 days, at least 12 days at least 15 days, at least 20 days, at least 25 days, at least 30 days, at least 1 month, at least 1.5 months, at least 2 months, at least 2.5 months, at least 3 months, at least 3.5 months, at least 4 months, at least 4.5 months, at least 5 months, at least 5.5 months, at least 6 months, at least 6.5 months, at least 7 months, at least 7.5 months, at least 8 months, at least 8.5 months, at least 9 months, at least 9.5 months, at least 10 months, at least 10.5 months, at least 11 months, at least 11.5 months, or at least 12 months in advance of a forecasted spread. In various embodiments, the predictive model predicts the likelihood of spread between 0.5 months and 6 months, between 1 month and 5 months, between 1.5 months and 4.5 months, between 2 months and 4 months, between 2.5 months and 3.5 months, or between 2.75 months and 3.25 months in advance of a forecasted spread.

In various embodiments, the model deployment module 125 deploys the predictive model to analyze values of features from a preceding time window. For example, as shown in FIG. 1B, Window 9 may be the immediately preceding time window to the current time T=0 (e.g., Window 9 is between T=−3 months and T=0 months). Therefore, the feature extraction module 115 may extract values of features from the surveillance data of Window 9 and the model deployment module 125 deploys the predictive model to analyze the values of the features from the surveillance data of Window 9. Thus, the predictive model can predict the likely spread of the mutation in the future based on the current values of features in Window 9.

In various embodiments, the predictive model outputs a score representing a probability of whether the mutation is likely to spread in the future. In various embodiments, the score is a continuous value, such as a continuous value between “0” and “1.” In such embodiments, the score is the probability of future spread of the mutation.

In various embodiments, the model deployment module 125 determines whether the mutation of the pathogen will spread according to the predicted score. In some embodiments, the model deployment module 125 compares the score outputted by the predictive model to a reference score. In various embodiments, the reference score is a pre-defined threshold score. In various embodiments, the pre-defined threshold score is any of 0.1, 0.2, 0.3, 0.4, 0.5, 0.6, 0.7, 0.8, or 0.9. In particular embodiments, the pre-defined threshold score is 0.5. In particular embodiments, the pre-defined threshold score is 0.6. In particular embodiments, the pre-defined threshold score is 0.7.

In various embodiments, the reference score is a score corresponding to prior mutations that have either spread or not spread. Therefore, by comparing the score outputted by the predictive model to a reference score of mutations that have either spread or not spread, the model deployment module 125 can determine whether the mutation is more like a prior mutation that spread, or a prior mutation that did not spread. For example, if the model deployment module 125 determines that the score outputted by the predictive model is statistically significantly different (e.g., p-value<0.05) from a reference score of mutations that previously spread, then the model deployment module 125 can identify the mutation as not likely to spread. As another example, if the model deployment module 125 determines that the score outputted by the predictive model is statistically significantly different (e.g., p-value<0.05) from a reference score of mutations that did not previously spread, then the model deployment module 125 can identify the mutation as likely to spread.

In various embodiments, the predictive model achieves a performance metric when predicting likelihood of spread for a mutation. For example, the predictive model may achieve an area under the receiving operating curve (AUROC) value of at least 0.6, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, at least 0.74, at least 0.75, at least 0.76, at least 0.77, at least 0.78, at least 0.79, at least 0.80, at least 0.81, at least 0.82, at least 0.83, at least 0.84, at least 0.85, at least 0.86, at least 0.87, at least 0.88, at least 0.89, at least 0.90, at least 0.91, at least 0.92, at least 0.93, at least 0.94, at least 0.95, at least 0.96, at least 0.97, at least 0.98, or at least 0.99.

In various embodiments, the predictive model may achieve an area under the receiving operating curve (AUROC) value of at least at least 0.6, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, at least 0.74, at least 0.75, at least 0.76, at least 0.77, at least 0.78, at least 0.79, at least 0.80, at least 0.81, at least 0.82, at least 0.83, at least 0.84, at least 0.85, at least 0.86, at least 0.87, at least 0.88, at least 0.89, at least 0.90, at least 0.91, at least 0.92, at least 0.93, at least 0.94, at least 0.95, at least 0.96, at least 0.97, at least 0.98, or at least 0.99 when predicting 1 month in advance of a forecasted spread. In particular embodiments, the predictive model achieve an AUROC value of at least 0.90 for predicting 1 month in advance of a forecasted spread.

In various embodiments, the predictive model may achieve an area under the receiving operating curve (AUROC) value of at least at least 0.6, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, at least 0.74, at least 0.75, at least 0.76, at least 0.77, at least 0.78, at least 0.79, at least 0.80, at least 0.81, at least 0.82, at least 0.83, at least 0.84, at least 0.85, at least 0.86, at least 0.87, at least 0.88, at least 0.89, at least 0.90, at least 0.91, at least 0.92, at least 0.93, at least 0.94, at least 0.95, at least 0.96, at least 0.97, at least 0.98, or at least 0.99 when predicting 2 months in advance of a forecasted spread. In particular embodiments, the predictive model achieves an AUROC value of at least 0.85 for predicting 2 months in advance of a forecasted spread.

In various embodiments, the predictive model may achieve an area under the receiving operating curve (AUROC) value of at least at least 0.6, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, at least 0.74, at least 0.75, at least 0.76, at least 0.77, at least 0.78, at least 0.79, at least 0.80, at least 0.81, at least 0.82, at least 0.83, at least 0.84, at least 0.85, at least 0.86, at least 0.87, at least 0.88, at least 0.89, at least 0.90, at least 0.91, at least 0.92, at least 0.93, at least 0.94, at least 0.95, at least 0.96, at least 0.97, at least 0.98, or at least 0.99 when predicting 3 months in advance of a forecasted spread. In particular embodiments, the predictive model achieves an AUROC value of at least 0.80 for predicting 3 months in advance of a forecasted spread.

In various embodiments, the predictive model may achieve an area under the receiving operating curve (AUROC) value of at least at least 0.6, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, at least 0.74, at least 0.75, at least 0.76, at least 0.77, at least 0.78, at least 0.79, at least 0.80, at least 0.81, at least 0.82, at least 0.83, at least 0.84, at least 0.85, at least 0.86, at least 0.87, at least 0.88, at least 0.89, at least 0.90, at least 0.91, at least 0.92, at least 0.93, at least 0.94, at least 0.95, at least 0.96, at least 0.97, at least 0.98, or at least 0.99 when predicting 4 months in advance of a forecasted spread. In particular embodiments, the predictive model achieves an AUROC value of at least 0.60 for predicting 4 months in advance of a forecasted spread. In particular embodiments, the predictive model achieves an AUROC value of at least 0.70 for predicting 4 months in advance of a forecasted spread. In particular embodiments, the predictive model achieves an AUROC value of at least 0.80 for predicting 4 months in advance of a forecasted spread. In particular embodiments, the predictive model achieves an AUROC value of at least 0.85 for predicting 4 months in advance of a forecasted spread. In particular embodiments, the predictive model achieves an AUROC value of at least 0.87 for predicting 4 months in advance of a forecasted spread.

Returning again to FIG. 1A, the spread prediction module 130 identifies a pathogen variant (e.g., a VOC or VOI) that is likely to spread based on one or more mutations that are predicted to spread. For example, the spread prediction module 130 can identify a pathogen variant (e.g., a VOC or VOI) that includes a combination of the mutations that are predicted to spread. By identifying a pathogen variant (e.g., a VOC or VOI) that is likely to spread before it is prevalent (e.g., prevalent in a country or across the world), this presents an opportunity to develop therapeutic interventions to address the pathogen variant before it spreads.

In various embodiments, the spread prediction module 130 may identify a pathogen variant (e.g., a VOC or VOI) with at least two, at least three, at least four, at least five, at least six, at least seven, at least eight, at least nine, at least ten, at least eleven, at least twelve, at least thirteen, at least fourteen, at least fifteen at least sixteen, at least seventeen, at least eighteen, at least nineteen, at least twenty, at least twenty five, at least thirty, at least thirty five, at least forty, at least forty five, at least fifty, at least fifty five, at least sixty, at least sixty five, at least seventy, at least seventy five, at least eighty, at least eighty five, at least ninety, at least ninety five, or at least a hundred mutations that are predicted by the predictive model to likely spread in the future.

In various embodiments, the spread prediction module 130 may identify a pathogen variant that is likely to spread (e.g., a VOC or VOI) based on prior pathogen variants. For example, the spread prediction module 130 may identify a pathogen variant that is likely to spread (e.g., a VOC or VOI) based on a prior VOC or VOI (e.g., a prior VOC or VOI specified by a public health agency (e.g., the Centers for Disease Control and Prevention (CDC)). In various embodiments, the spread prediction module 130 may identify a pathogen variant that is likely to spread (e.g., a VOC or VOI) as a prior VOC or VOI plus additional mutations that are predicted to spread (e.g., predicted to spread based on a predictive model). In various embodiments, the spread prediction module 130 may identify a pathogen variant that is likely to spread (e.g., a VOC or VOI) as a prior VOC or VOI plus additional mutations occur at a rate of at least a threshold percentage of the most prevalent variant in the lineage. In various embodiments, the threshold percentage is at least 50%, at least 55%, at least 60%, at least 65%, at least 70%, at least 75%, at least 80%, at least 85%, at least 90%, or at least 95%.

To provide an example, to date, the Omicron BA.2 variant remains the prevalent SARS-CoV-2 strain worldwide. By analyzing at least the most recent surveillance data relative to the Omicron BA.2 variant, the predictive model may identify one or more mutations that are likely to spread in the future. The spread prediction module 130 may identify a new Omicron variant with the identified one or more mutations such that the new Omicron variant is likely to be the prevalent strain in the future. One example is the Omicron BA.4/5 variant which is defined by the following mutations: T19I, Δ24-26, Δ27S, Δ69/70, G142D, V213G, G339D, S371F, S373P, S375F, T376A, D405N, R408S, K417N, N440K, L452R, S477N, T478K, E484A, F486V, Q498R, N501Y, Y505H, D614G, H6SSY, N679K, P681H, N764K, D796Y, Q954H, N969K. Thus, the spread prediction module 130 can identify the new Omicron variant (e.g., Omicron BA.4/5 variant) prior to actual spread of the new Omicron variant.

### Example Features

Methods disclosed herein involve identifying features of mutations and/or analyzing values of features of mutations for predicting whether mutations are likely to spread. Features of mutations may refer to a characteristic of a mutation. In various embodiments, features may be categorized in any one of epidemiology features, evolution features, transmissibility features, language model features, or immune features.

Generally, epidemiology features of a mutation describe characteristics of a mutation such as the distribution and frequency of a mutation. Example epidemiology features include the variant frequency (also referred to as mutation frequency), the fraction of unique haplotypes with the mutation, and the number of countries in which the mutation has appeared. In various embodiments, an epidemiology feature can be represented as a score. For example, an exemplary epidemiology feature may be herein referred to as an “Epi Score.” In various embodiments, the Epi Score represents the exponentially weighted mean rank across the other epidemiology features. For example, assuming the other epidemiology features of 1) variant frequency, 2) fraction of unique haplotypes, and 3) number of countries, the Epi Score can be calculated as follows: (i) calculating the percentile for each other epidemiology feature, (ii) exponentiating percentile to the power of 10, and (iii) averaging these exponentiated percentiles. The effect of this procedure is to assign highly differentiated weights to high rankings, and relatively small and similar weights to mutations that are not at the top of the list. Thus, use of the Epi score is particularly advantageous if measurements for lower-ranked entities are more noisy than higher ranked ones (e.g., as this increases the weights of higher rankings).

Evolution features refer to features describing the evolution of a mutation. Examples of evolution features include negative selection features, positive selection features, RNA structure features (e.g., Codon-SHAPE), and entropy features. Example positive selection features include parameters from fixed effects likelihood (FEL) and mixed effects model of evolution (MEME) models. Further details of positive selection features (e.g., FEL and MEME models) are described in Pond, S. L. K. et al. HyPhy 2.5-A Customizable Platform for Evolutionary Hypothesis Testing Using Phylogenies. Mol Biol Evol 37, 295-299 (2019), which is hereby incorporated by reference in its entirety. Codon-SHAPE features refer to RNA-SHAPE constraints, which is further described in Manfredonia, I. et al. Genome-wide mapping of therapeutically-relevant SARS-COV-2 RNA structures. Biorxiv 2020.06.15.151647 (2020), which is hereby incorporated by reference in its entirety. Entropy features refer to the Shannon entropy at each codon position for an amino acid site.

Transmissibility features refer to mutations that alter a protein of the pathogen, thereby impacting the transmissibility of the pathogen. In various embodiments, transmissibility features refer to mutations to either the receptor binding domain (RBD) or Spike protein. For example, a transmissibility feature may be referred to herein as a RBD expression change feature, which represents the change in RBD expression due to the mutation. As another example, a transmissibility feature may be referred to herein as an ACE2 binding change features, which represents the change in binding affinity for ACE2 due to the mutation. Further description of RBD expression change and ACE2 binding change is detailed in Starr, T. N. et al. Deep mutational scanning of SARS-COV-2 receptor binding domain reveals constraints on folding and ACE2 binding. Cell (2020), which is hereby incorporated by reference in its entirety.

Language model features generally refer to linguistic analogs of pathogen escape. For example, linguistic analogs can refer to viability and infectivity due to the mutation (e.g., referred to as grammaticality) and/or the evasiveness of a pathogen due to the mutation (e.g., referred to as semantic change). For example, a natural language processing (NLP) neural network can be implemented to derive features involving grammaticality and semantic change. Further details of grammaticality and semantic changes of mutations is described in Hie, B., Zhong, E. D., Berger, B. & Bryson, B. Learning the language of viral evolution and escape. Science 371, 284-288 (2021), which is hereby incorporated by reference in its entirety.

Immune features generally refer to the impact of a mutation on the immune response. Example immune features include a CD8 epitope escape feature, CD8 response features, CD4 response features, antibody binding score features, and maximum escape fraction in vitro. The CD8 epitope escape feature refers to the frequency of a mutation in cytotoxic lymphocyte (CTL) epitopes. Further details of the CD8 epitope escape is described in Agerer, B. et al. SARS-COV-2 mutations in MHC-I-restricted epitopes evade CD8+ T cell responses. Sci Immunol 6, eabg6461 (2021), which is hereby incorporated by reference in its entirety. The CD8 response feature refers to the percent and average CD8+ T-cell response to an epitope with the mutation in patients. The CD4 response feature refers to the percent and average CD4+ T-cell response to an epitope with the mutation in patients. Further details of the CD8 response and CD4 response are described in Tarke, A. et al. Comprehensive analysis of T cell immunodominance and immunoprevalence of SARS-CoV-2 epitopes in COVID-19 cases. Cell Reports Medicine 2, 100204 (2021), which is hereby incorporated by reference in its entirety. The maximum escape fraction in vitro refers to the maximum escape fraction across all conditions for that mutation. Further details of the maximum escape fraction in vitro is described in Greaney, A. J. et al. Complete Mapping of Mutations to the SARS-COV-2 Spike Receptor-Binding Domain that Escape Antibody Recognition. Cell Host Microbe 29, 44-57.e9 (2021), which is hereby incorporated by reference in its entirety.

The antibody binding score features represent the estimated percent contribution of a site to binding of the indicated antibody, as estimated by Molecular Operating Environment (MOE). Example antibodies can be therapeutic antibodies developed for binding to pathogens, examples of which include S309, S304, S2M11, S2H14, S2H13, S2E12, S2A4, REGN10987, REGN10933, S2M28, LY-CoV555, LY-COV016, S2L28, CT-P59, BD-368-2, and Brii-196.

Specifically, antibody binding scores can be calculated using Molecular modeling software MOE²⁵ (v2019.0102). To produce the antibody binding score, the first step may involve calculating pairwise binding energies (the sum of van der Waals, ionic, aromatic, and hydrogen-bond interactions) between each residue in the antigen epitope and each residue in the corresponding antibody Fab paratope, including all residues within a cutoff distance of 5.0 Å from the epitope/paratope interface. Structures are prepared prior to these calculations using the structure preparation, protonation and energy minimization steps in MOE, with default settings. The binding energies of each epitope residue that interacted with multiple Fab residues can be added together and the percentage of the binding energy contributed by each epitope residue to the total binding energy was calculated. When more than one copy of the complex is present in the asymmetric unit, binding energy contributions can be averaged across all copies. An overall binding energy per site is calculated as the max score across all antibodies.

Additional details and examples of features are described below in Tables 1 and 7.

### Example Process for Predicting Pathogen Spread

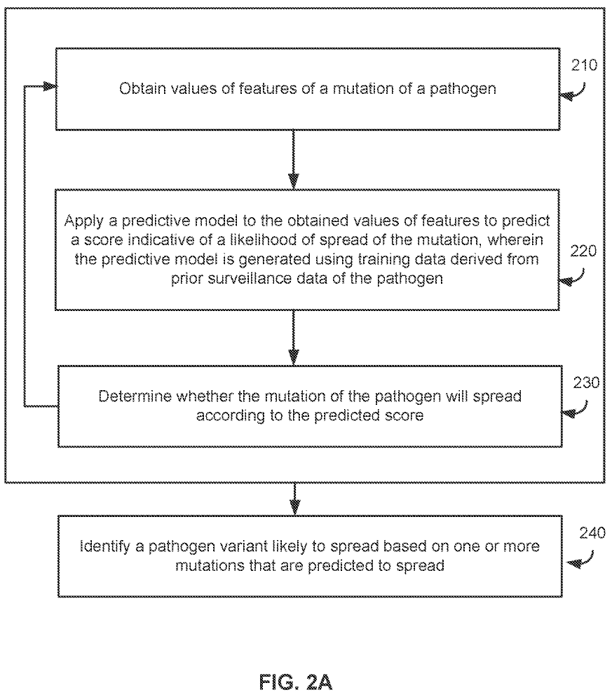

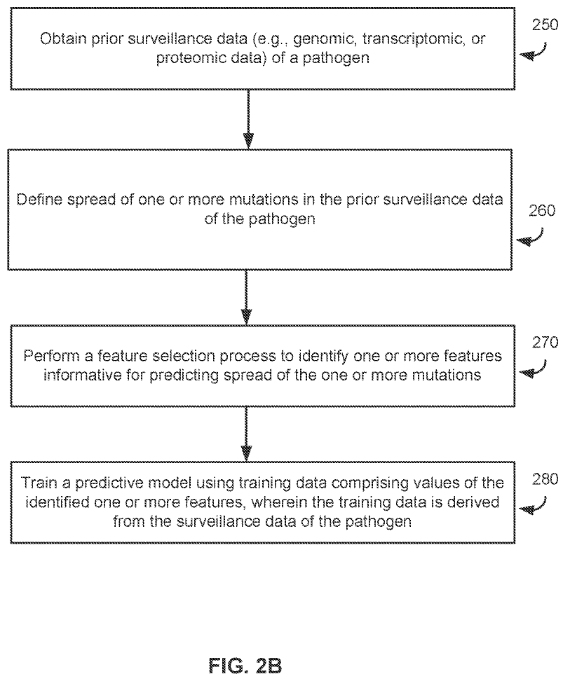

FIG. 2A is an example flow process for predicting whether a mutation of a pathogen will spread, in accordance with an embodiment. Step 210 involves obtaining values of features of a mutation of the pathogen. The phrase “obtaining values of features of a mutation” encompasses obtaining values of features (e.g., via a feature extraction process) from prior surveillance data related to the mutation. The phrase also encompasses receiving values of features, e.g., from a third party that has performed a feature extraction process on surveillance data to determine values of features.

As discussed herein, the features can include one or more different categories of features, examples of which include epidemiology features, evolution features, transmissibility features, language model features, or immune features. In particular embodiments, step 210 involves obtaining values of features comprising epidemiology features. In particular embodiments, step 210 involves obtaining values of only epidemiology features. In particular embodiments, step 210 involves obtaining values of features comprising evolution features, immune features, and/or transmissibility features. In particular embodiments, step 210 involves obtaining values of features comprising each of epidemiology features, evolution features, transmissibility features, language model features, and immune features

Step 220 involves applying a predictive model to the obtained values of features to predict a score indicative of a likelihood of spread of the mutation. In various embodiments, step 220 involves applying the predictive model to analyze values of features comprising epidemiology features. In particular embodiments, step 220 involves applying the predictive model to analyze only values of epidemiology features. In various embodiments, step 230 involves applying the predictive model to analyze values of features comprising evolution features, immune features, and/or transmissibility features. In various embodiments, step 230 involves applying the predictive model to analyze values of features comprising each of epidemiology features, evolution features, transmissibility features, language model features, and immune features. As described herein, the predictive model is generated using training data derived from prior surveillance data of the pathogen. For example, the prior surveillance data of the pathogen can include one or more prior waves involving the pathogen during which one or more mutations of the pathogen enabled the pathogen to spread.

Step 230 involves determining whether the mutation of the pathogen will spread according to the predicted score determined by the predictive model. In various embodiments, if the predicted score for the mutation is above a threshold value, the mutation is predicted to likely spread. In various embodiments, if the predicted score for the mutation is below a threshold value, the mutation is predicted to not spread.

As shown in FIG. 2A, after step 230, the flow process may begin again at step 210 for an additional mutation. Thus, steps 210, 220, and 230 can be repeated for an additional mutation of the pathogen to predict whether the additional mutation is likely to spread. The steps 210, 220, and 230 can be iteratively repeated for further additional mutations of the pathogen.

Step 240 involves identifying a pathogen variant that is likely to spread based on one or more mutations that were determined, over repeated iterations at step 230, to likely spread. For example, the pathogen variant may harbor the one or more mutations. In various embodiments, step 240 is performed prior to actual spread of the pathogen variant (e.g., at least 1 month, at least 2 months, at least 3 months, at least 4 months, at least 5 months, at least 6 months, at least 7 months, at least 8 months, at least 9 months, at least 10 months, at least 11 months, or at least 12 months prior to actual spread of the pathogen variant). Thus, identification of the pathogen variant is useful for developing possible therapeutic interventions to prevent the upcoming spread of the pathogen variant.

### Example Process for Training a Predictive Model

FIG. 2B is an example flow process for training predictive model for use in predicting pathogenic spread. Step 250 involves obtaining prior surveillance data for a pathogen. In various embodiments, the prior surveillance data comprises genomic, transcriptomic, or proteomic surveillance data of the pathogen.

Step 260 involves defining spread of one or more mutations in the prior surveillance data of the pathogen. In various embodiments, spread of a mutation is defined according to an absolute frequency or a fold change of the frequency of the mutation present in one or more geographical locations (e.g., worldwide or one or more countries). In various embodiments, spread of a mutation is defined according to a fold change in frequency of the mutation across time windows.

Step 270 involves performing a feature selection process to identify one or more features informative for predicting the defined spread of the one or more mutations. In various embodiments, the identified features may include features from one or more categories of features, examples of which include epidemiology features, evolution features, transmissibility features, language model features, or immune features. In particular embodiments, step 270 involves performing a feature selection process that identifies at least epidemiology features that are informative for predicting the defined spread of the one or more mutations.

Step 280 involves training a predictive model using training data comprising values of the identified one or more features. Here, the training data is derived from the obtained prior surveillance data of the pathogen. In various embodiments, the training data is derived from prior surveillance data comprising data at least 1 month, at least 2 months, at least 3 months, at least 4 months, at least 5 months, at least 6 months, at least 7 months, at least 8 months, at least 9 months, at least 10 months, at least 11 months, or at least 12 months prior to the spread of one or more mutations of the pathogen. Thus, the predictive model is trained to predict whether a mutation is likely to spread or not spread based on the values of the features of the training data.

### Example Pathogens and Diseases

Disclosed herein are methods useful for predicting likely spread of particular pathogens with one or more mutations. In various embodiments, a pathogen refers to any one of a virus, bacteria, fungus, or a parasite. In particular embodiments, the pathogen is a virus. In various embodiments, the pathogen is capable of causing any one of the following diseases: severe acute respiratory syndrome-related coronavirus (SARS), severe acute respiratory syndrome coronavirus 2 (SARS-COV-2), influenza, Ebola, human immunodeficiency virus (HIV), Hepatitis B virus (HBV), Hepatitis C virus (HCV), Human papillomavirus (HPV), tuberculosis, or herpes simplex virus infection (HSV). In particular embodiments, the pathogen is a SARS-COV-2 virus that causes SARS-COV-2. In particular embodiments, the pathogen is an influenza virus (Influenza A virus, influenza B virus, influenza C virus, or influenza D virus) that causes influenza.

The pathogen may include one or more mutations. Such mutations represent genetic changes that may occur spontaneously as the pathogen replicates. In various embodiments, the one or more mutations confer an advantage to the pathogen in comparison to other mutations. In various embodiments, a pathogen can be characterized according to the one or more mutations that are present. In various embodiments, various lineages of pathogens can be determined or characterized based on the presence of one or more mutations. For example, referring to the Omicron variant of SARS-COV-2, the Omicron parent, presumably identified in 2021, includes 39 mutations shared across its sublineages (e.g., BA.1, BA.2, and BA.3 Omicron subvariants). The BA. 1 subvariant contains an additional 20 mutations on top of the 39 of the Omicron parent, the BA.2 subvariant contains an additional 27 mutations, and the BA.3 subvariant contains an additional 13 mutations. Thus, the lineage of the SARS-COV-2 Omicron variant can be defined according to the presence of different mutations.

In various embodiments, the one or more mutations may occur at a particular location of protein that leads to a change in the protein of the pathogen. For example, referring again to SARS-CoV-2, a mutation may be present in a receptor-binding domain (RBD) of SARS-COV-2 virus. In various embodiments, a mutation may be present in a Spike protein, signal peptide, or N-terminal domain of the SARS-COV-2 virus.

As described in further detail herein, example SARS-COV-2 mutations useful for predicting whether SARS-COV-2 is likely to spread include one or more of: D614G, N501Y, P681H, H69-, V70-, T716I, Y144-, S982A, D1118H, A570D, A222V, E484K, L18F, A701V, L5F, L452R, T95I, Q677H, S477N, D80A, N439K, L242-, S98F, K417N, A243-, D215G, L241-, D138Y, H655Y, P26S, V1176F, S13I, T478K, T1027I, Q675H, D253G, W152C, A67V, S494P, V143-, R190S, T20N, P681R, G142-, K417T, T732A, S939F, G769V, M153T, A262S, A845S, D138H, K1191N, L189F, F888L, L141-, Q52R, V1228L, S12F, A1078S, T572I, P272L, L54F, H49Y, A688V, V1264L, L176F, V772I, A653V, E583D, W152L, T20I, A522S, N501T, Q613H, S640F, W258L, A520S, V622F, G1219V, D80Y, N679K, T859N, G75V, T22I, M1237I, Q675R, M1229I, M153I, T859I, T76I, P812S, P812L, N440K, D796Y, W152R, D1163Y, P1263L, P1162S, F157L, G1219C, D936Y, F490S, A1020S, S221L, S254F, E484Q, V367F, T29I, G181V, F157S, S255F, A27S, A879S, T11171, T7911, Q1071L, Y144F, A899S, E1202Q, V308L, P1162L, P384L, P809S, S704L, H1101Y, H245Y, D950H, P26L, S256L, P9L, L938F, S1252F, K1073N, D796H, T19I, T3071, A706V, T547I, V1104L, D215Y, L822F, M1771, S940F, A684V, T51I, L452Q, K558N, E96D, S94F, V1122L, L1063F, Y144V, D1118Y, G142D, T240I, A27V, G1124V, E154K, D80G, 168-, H69Y, L216F, T323I, D936N, T1273-, V70I, S6891, Y1272-, T678I, A67S, H1271-, A672V, T299I, A846V, F140-, L1270-, V1268-, S71-, K1269-, A771S, A1070V, A1020V, A892V, C1247F, P330S, F565L, Q414K, D215H, G1267-, 1818V, M731I, G446V, R214L, D1257-, Q1071H, E1262-, P1263-, K1266-, and V1264-.

In particular embodiments, SARS-COV-2 variants include mutations relative to the Wuhan reference strain (NCBI Ref: 43740568) and are designated as follows:
Alpha variant (alterations: 469/70, 4144, N501Y, A570D, D614G, P681H, T716I, S982A, D111SH)
Beta variant (alterations: L18F, D80A, D215G, Δ242-244, R246I, K417N, E484K, N501Y, D614G, A701V)
Delta variant (alterations: T19R, G142D, E156G, A157/158, K417N, L452R, T478K, D614G, P681R, D950N)
Omicron BA.1 variant (alterations: A67V, 469/70, T95I, G142D, A143-145, Δ211, L212I, ins214EPE, G339D, S371L, S373P, S375F, K417N, N440K, G446S, S477N, T478K, E484A, Q493R, G496S, Q498R, NSO1Y, YSOSH, T547K, D614G, H65SY, N679K, P681H, N764K, D796Y, N856K, Q954H, N969K, L981F)
Omicron BA.2 variant (alterations: T19I, Δ24-26, A27S, G142D, V213G, G339D, S371F, S373P, S375F, T376A, D405N, R408S, K417N, N440K, S477N, T478K, E484A, Q493R, Q498R, N501Y, Y505H, D614G, H655Y, N679K, P681H, N764K, D796Y, Q954H, N969K)
Omicron BA.4/5 variant (alterations: T191, Δ24-26, A27S, Δ69/70, G142D, V213G, G339D, S371F, S373P, S375F, T376A, D405N, R408S. K417N, N440K, L452R, S477N, T478K, E484A, F486V, Q498R, N501Y, Y505H, D614G, H655Y, N679K, P681H, N764K, D796Y, Q954H, N969K)

As another example, referring to influenza, a mutation may be present in any of surface proteins of the influenza virus, the influenza hemagglutinin (HA) protein, and/or viral neuraminidase. Thus, such mutations present in the influenza virus can be useful for predicting whether the influenza virus is likely to spread in the future.

### Non-transitory Computer Readable Medium

Also provided herein is a computer readable medium comprising computer executable instructions configured to implement any of the methods described herein. In various embodiments, the computer readable medium is a non-transitory computer readable medium. In some embodiments, the computer readable medium is a part of a computer system (e.g., a memory of a computer system). The computer readable medium can comprise computer executable instructions for implementing a machine learning model for the purposes of predicting a clinical phenotype.

### Computing Device

The methods described above, including the methods for predicting mutations that are likely to spread, are, in some embodiments, performed on a computing device. Examples of a computing device can include a personal computer, desktop computer laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, and the like.

In various embodiments, the different methods described above in relation to FIGS. 1, 2A, 2B such as the methods for predicting mutations that are likely to spread, may be implemented using one or more computing devices. For example, the pathogen prediction system 110 may be embodied as one or more computing devices.

The methods for predicting mutations that are likely to spread can be implemented in hardware or software, or a combination of both. In one embodiment, a non-transitory machine-readable storage medium, such as one described above, is provided, the medium comprising a data storage material encoded with machine readable data which, when using a machine programmed with instructions for using said data, is capable of displaying any of the datasets and execution and results e.g., a prediction of one or more mutations that are likely to spread or not spread.

Embodiments of the methods described above can be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high-level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information.

“Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

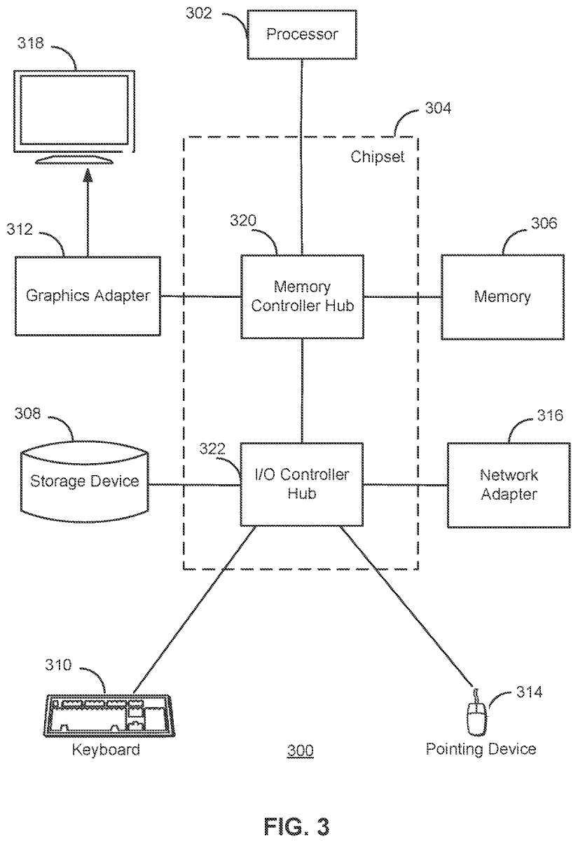

FIG. 3 illustrates an example computing device 300 for implementing methods and systems described in FIGS. 1, 2A, and 2B. In some embodiments, the computing device 300 includes at least one processor 302 coupled to a chipset 304. The chipset 304 includes a memory controller hub 320 and an input/output (I/O) controller hub 322. A memory 306 and a graphics adapter 312 are coupled to the memory controller hub 320, and a display 318 is coupled to the graphics adapter 312. A storage device 308, an input interface 314, and network adapter 316 are coupled to the I/O controller hub 322. Other embodiments of the computing device 300 have different architectures.

The storage device 308 is a non-transitory computer-readable storage medium such as a hard drive, compact disk read-only memory (CD-ROM), DVD, or a solid-state memory device. The memory 306 holds instructions and data used by the processor 302. The input interface 314 is a touch-screen interface, a mouse, track ball, or other type of input interface, a keyboard, or some combination thereof, and is used to input data into the computing device 300. In some embodiments, the computing device 300 may be configured to receive input (e.g., commands) from the input interface 314 via gestures from the user. The graphics adapter 312 displays images and other information on the display 318. The network adapter 316 couples the computing device 300 to one or more computer networks.

The computing device 300 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 308, loaded into the memory 306, and executed by the processor 302.

The types of computing devices 300 can vary from the embodiments described herein. For example, the computing device 300 can lack some of the components described above, such as graphics adapters 312, input interface 314, and displays 318. In some embodiments, a computing device 300 can include a processor 302 for executing instructions stored on a memory 306.

### EXAMPLES

### Example 1: Defining and Predicting Spread of Mutations

### I. Defining Spreading Mutations

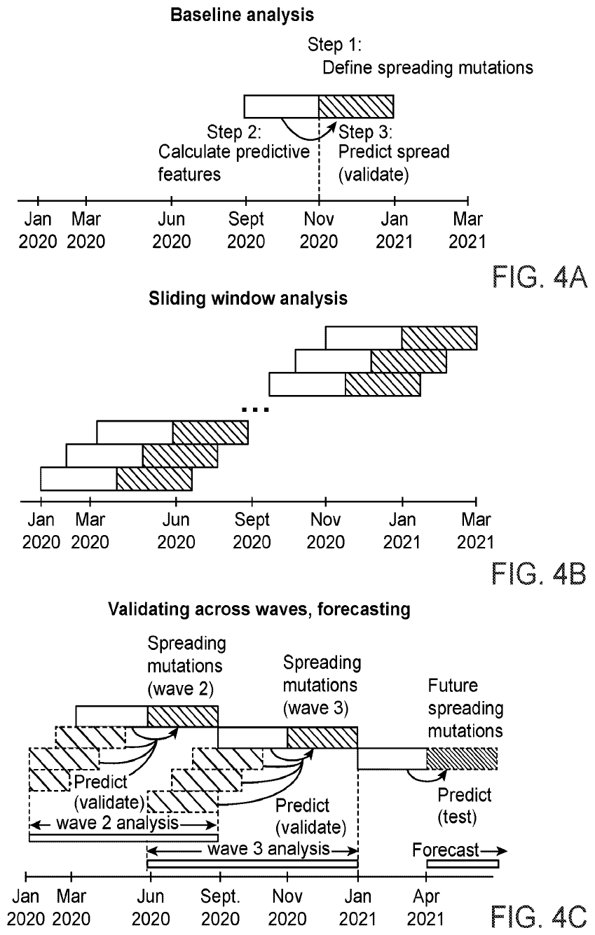

For the purpose of developing the models, spreading amino acid mutations were defined as a specified fold change in frequency across multiple countries, comparing time windows before and after a chosen date. Over 900,000 SARS-COV-2 Spike sequences available from GISAID² as of Apr. 24, 2021 were analyzed to characterize mutational spread within recognized and potential VOCs globally and regionally within the United States. A baseline analysis involved analyzing growth during the third wave of the pandemic (the three months after November 1; FIGS. 4A and 4C), using the three months prior as reference. Specifically, FIGS. 4A-4C depict the baseline study design. (A) The core analysis included three steps. First, a working definition for spreading mutations was created. Second, features that can predict future spread were created using a window of prior data. Third, models were constructed on training data, predictions of future spread were generated, and the results were then interpreted. (B) For the top univariate predictors, their performance over time was examined using a sliding window approach. (C) For wave 2 and wave 3, the same steps as A were conducted: defining spreading mutations, calculating predictive features, and predicting spread. To assess the possible lead time provided by the model, predictive features at 1, 2, 3, and 4 months in advance were calculated. To query robustness, predictions were analyzed for both wave 2 and wave 3. Finally, predictive features were generated based on contemporary data and the model was deployed to forecast the mutations that could spread in the future.

Fisher's exact test was used for frequency fold-change per country, adjusted for multiple comparisons, to identify a list of potentially spreading mutations. Within each country, the number of sequences containing the mutation of interest were tabulated, versus those that did not; in the three months before and after a date of interest (FIG. 4A). This was summarized as a 2×2 table per country, from which a fold change was calculated and an associated comparison-adjusted p-value. Mutations with a significant adjusted p-value from any country were retained. The number of comparisons for the adjustment was conservatively defined as the number of countries times the number of observed mutations worldwide. To account for violations in test assumptions (e.g., correlated counts due to biased sampling), and to enrich for the most concerning mutations, this set was further filtered using the following empirical criteria: (i) a fold change (FC) from baseline of at least 10.0 in at least one country, (ii) FC of at least 2.0 across three or more countries, (iii) a minimum global frequency of 0.1% in the later time window. Sequences used to calculate fold change from baseline and minimum frequency were all collected after those used for model training or feature calculation, with no overlap or interleaving between the two datasets.

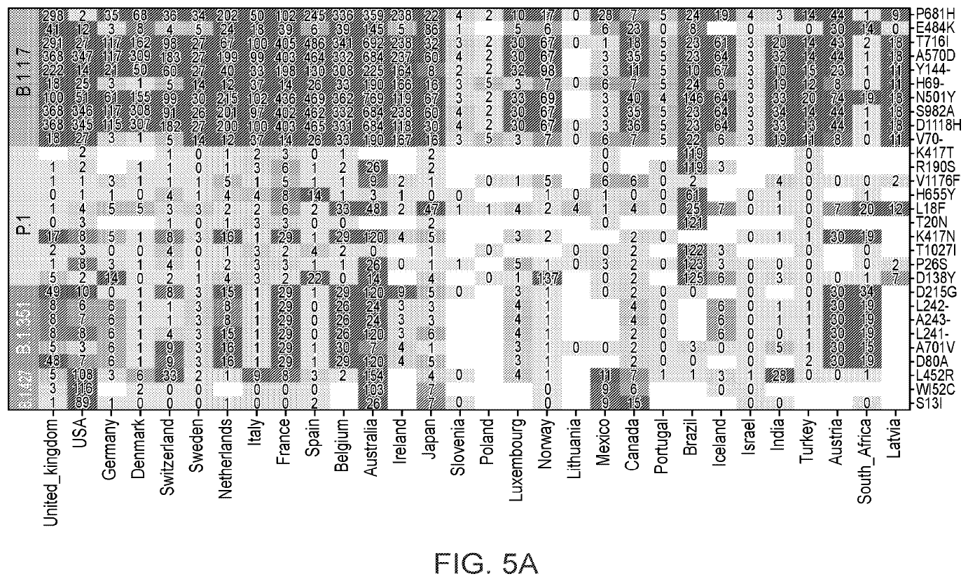

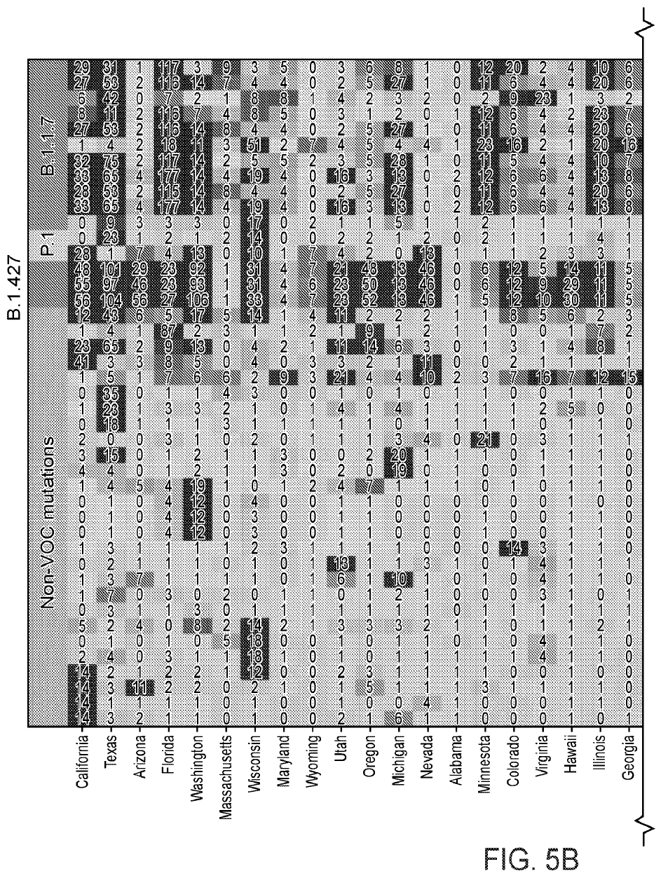

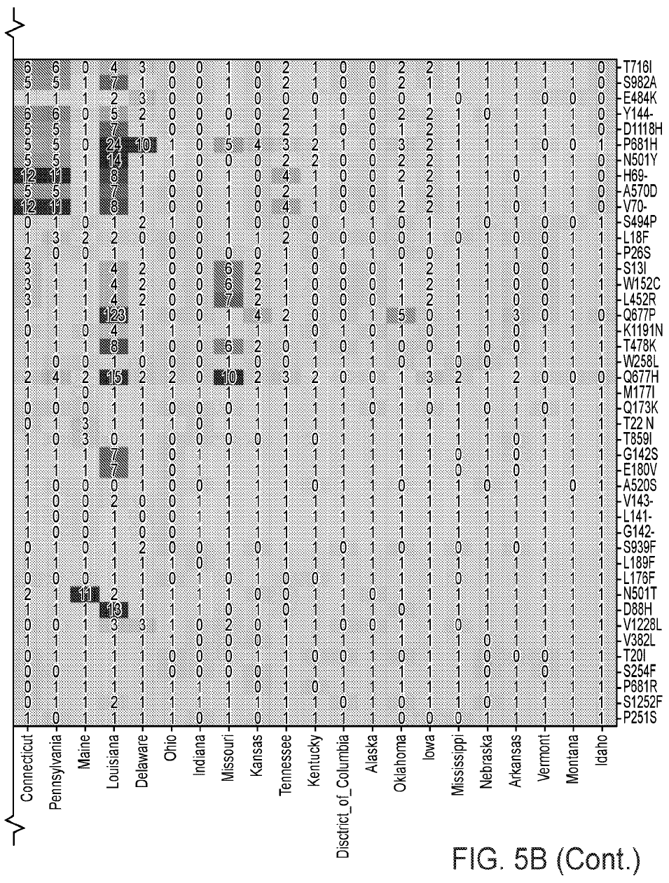

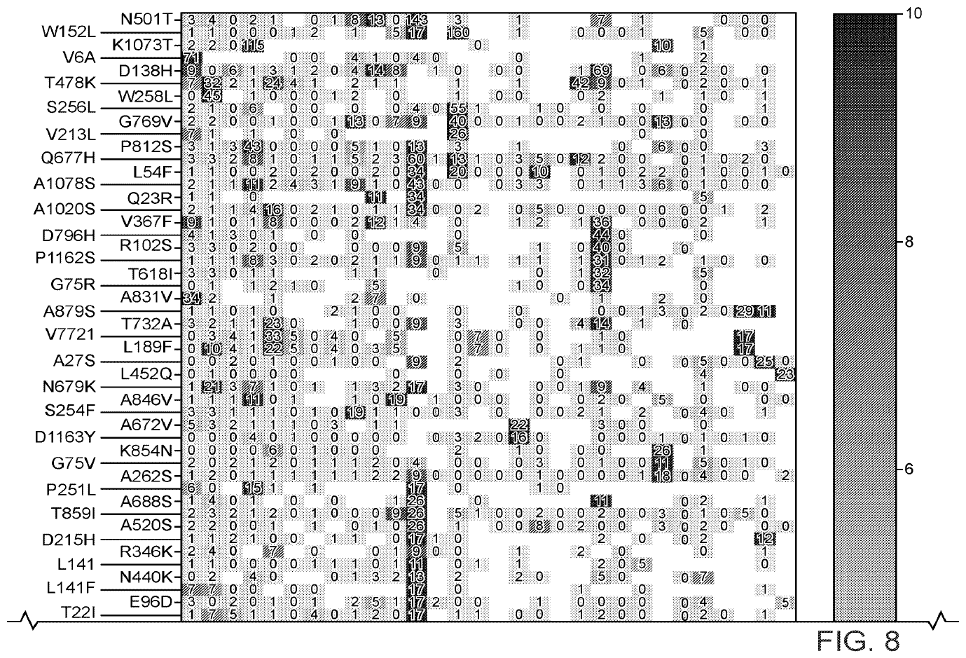

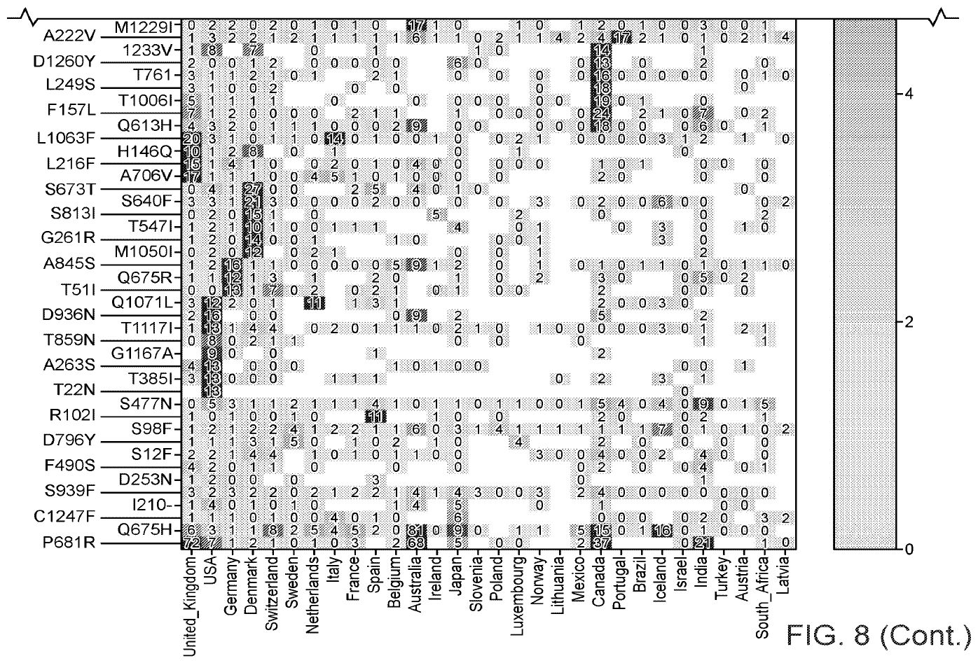

This definition of spreading mutations captured the current expansion of VOI/VOCs globally (FIG. 5A) as well as the growth of a number of lesser-known mutations (FIG. 8). Specifically, FIGS. 5A and 5B describe spreading mutations in the third wave. The working definition of spreading mutations captures the expansion of variants of concern during wave 3 at the country level (e.g., FIG. 5A) and at the state level within the United States (e.g., FIG. 5B). Leading variants of concern are denoted by colored bars on the left-hand side. Green: UK B.1.1.7; Yellow: Brazil P.1; Blue: South African B.1.351; Pink: California B.1.427/B.4.429. The emergence of E484K in association with B.1.1.7 is depicted in panel B. The California B.1.427/B.4.429 is observed across multiple US states. Previously unidentified potential spreading mutations are denoted by a beige colored bar on the left-hand side (e.g., “Non-VOC mutations”). The x-axis (countries or states) are ordered left to right according to decreasing number of GISAID submissions being represented. FIG. 8 shows all non-canonical variants highlighted for the Wave 3 analysis. Rows are clustered by relative risk profiles across countries. Columns are sorted by the amount of sequencing data. A table view for all mutations can be found in Table 8.

For example, identified mutations include R346K and V367F mutations, which increase Spike expression and contribute to immune escape¹⁴. V367F frequency increased at least 8× in the United Kingdom and Switzerland, 12× in Spain, and 36× in Canada. R346K increased 7× in Switzerland, 8× in Austria, and 21× in Chile. The broadest geographic increases were observed for P681R, which increased over 4× in 15 countries, and over 20× in 7 countries. P681R adds a basic amino acid adjacent to the Spike furin cleavage site and enhances the fusion activity of Spike in vitro¹⁹. This mutation is now dominant in the B. 1.617 lineage first detected in India. Thus, increases in the frequency of this mutation were detected well before the current wave of VOC-associated disease in India.

Furthermore, methods involve detecting additional spreading mutations (N501T, D138H, and W152L) at sites in the Spike already associated with another spreading mutation (N501Y, D138Y and W152C). N501T, like N501Y, also improves ACE2 binding of Spike²⁰. Together, these data indicate that the definition of globally spreading mutations in wave 3 of the pandemic surfaces pandemic-relevant changes in Spike protein function.

Next, regional patterns of mutation spread within the United States (FIG. 5B) were analyzed, such as the spread of both UK B.1.1.7 and California B.1.427/B.1.429 VOC mutations. Most of the increase in VOI/VOC mutations was observed in 14/50 states. Michigan, Florida, and Texas showed the most pronounced fold changes in mutations. Additionally, there was a set of less well-known mutations that appeared to be spreading in some regions (FIG. 5B). For example, T478K expanded over 60× in Texas (and 41× in neighboring Mexico). This mutation also increased at least 10× in Washington, California, and Oregon. T478K increases in vitro Spike expression and ACE2 binding¹⁴

Additionally, some mutations (e.g., A222V) are declining globally. However, per-country breakdowns demonstrated that A222V was still increasing in frequency in some countries, where other perhaps more fit mutations had not yet become entrenched. Using the interpretable statistical criteria therefore successfully identified the dynamics of mutations in VOCs and detected the spread of lesser known mutations globally and within the United States. 14

### II. Predicting Spreading Mutations

Methods further involve determining features of amino acid mutations that are useful for predicting their emergence in successful viral variants. This analysis itself can be viewed as a type of validation. An accurate forecast of future events can involve both a well-defined event inputs to the prediction that contain substantial information about the outcome of interest. For example, if in vitro measures of viral fitness accurately predict spreading mutations, this is supportive evidence for the definition of spreading mutations, the input data quality, and the predictive methodology.

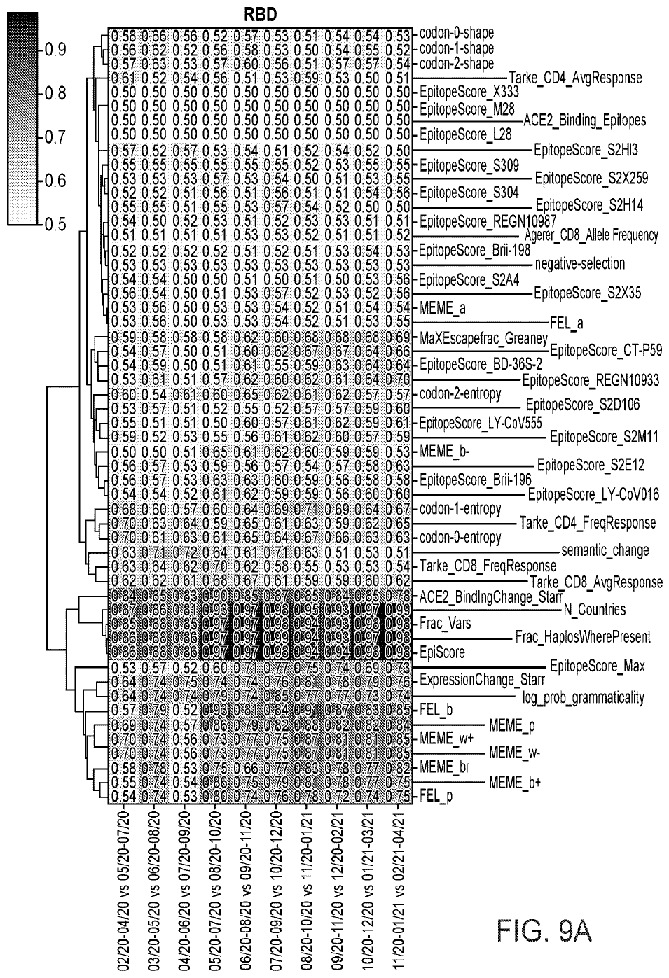

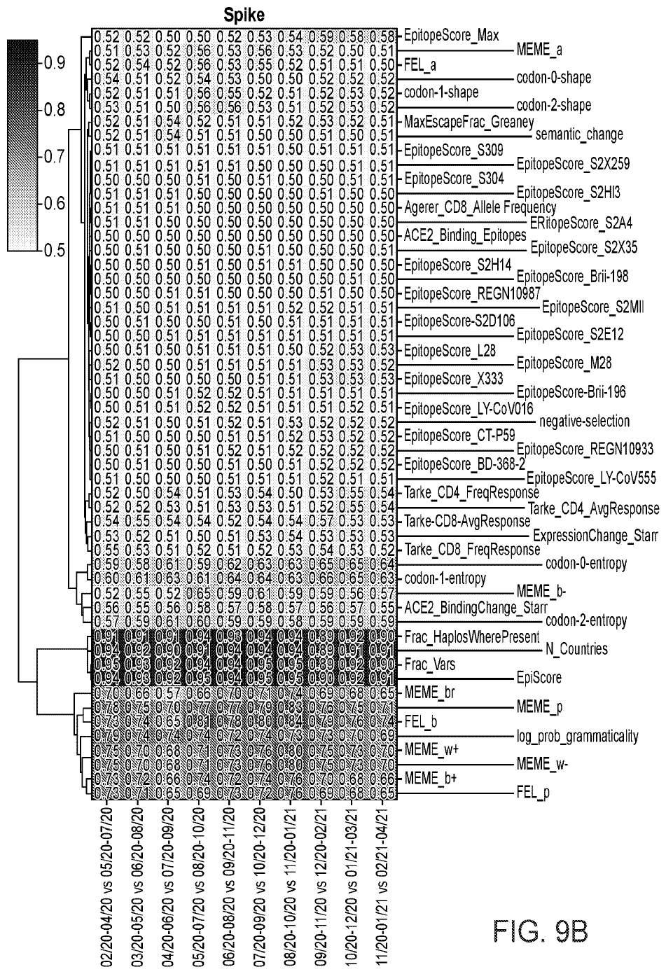

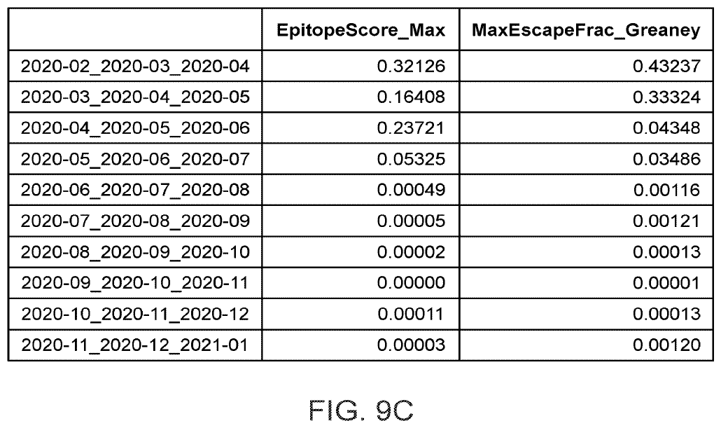

The predictiveness of individual features, and sets of features, were analyzed. These features were grouped together by the type of information they convey (Table 1 and Table 7). Specifically, features were categorized into the following groups: immunity, transmissibility, evolution, language model, and epidemiology. Of these groups, SARS-COV-2 evolution, language model, and epidemiology variables were further categorized as “GISAID-based”, because they are produced from the same input of the GISAID database. Here, methods involved testing performance for predicting mutation spread during the third wave of the pandemic, which was defined as the three months after November 1^{st}, using only data from the preceding three months. To assess robustness and ascertain changes in evolutionary dynamics, a similar examination of predictive performance in the second wave (June-August 2020) was performed, and more generally, in sliding windows across all periods of the pandemic. This enabled the determination of which features of a given mutation cause it to be more likely to spread, and whether this information could reliably be used to predict the spread of mutations several months in advance. Features from all groups were predictive of mutation spread, but the strength of their associations varied widely (FIG. 9A and FIG. 9B). Specifically, FIGS. 9A-9C show changes in predictiveness over time. Trends in RBD (FIG. 9A) and Spike (FIG. 9B) AUROC for all variables, over 10 sliding window periods. Baseline analysis ROCs correspond to the 08/20-10/20 vs 11/20-01/21 analysis. FIG. 9C shows the p-values for difference from random prediction over time, for the immune escape variables. Explanations of variable names can be found in Table 7.

### III. Assessment of Individual Features

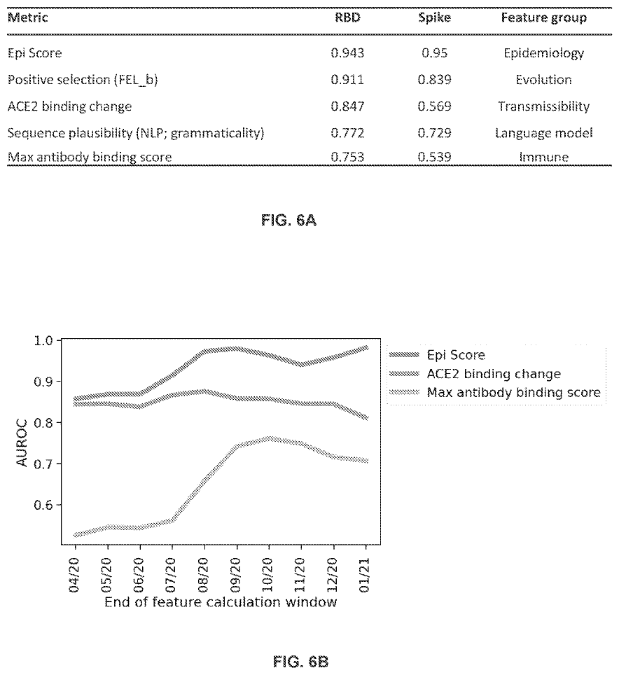

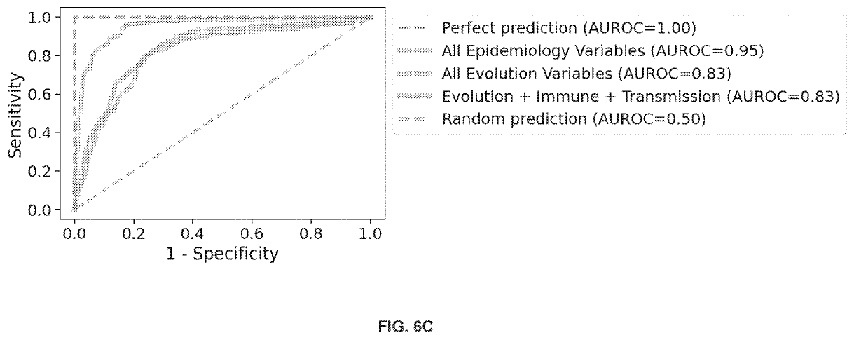

To predict spreading mutations from individual features, mutations were ranked directly using each feature with no model fitting step. Since the Receptor Binding Domain (RBD) region of Spike had the most complete data, analysis began there. FIGS. 6A-C shows the top predictors of mutation spread. Each variable was used to directly rank mutations for future spread. FIG. 6A shows the most predictive variables within each feature group (see Table 1 and Table 4), ranked by performance within the receptor binding domain (RBD), where the most data are available. Scores are Area Under the Receiver Operating Characteristic curve (AUROC). FIG. 6B shows RBD classification accuracy over time for the top GISAID-based feature (Epi score), and the top transmission and immune variables (Table 1). AUROCs in panel B are smoothed with a rolling window of two analysis periods. FIG. 6C shows performance for identifying which mutations will spread during the baseline analysis period (see FIG. 4A).

Within the RBD, ACE2 binding affinity was a slightly better predictor of mutation spread (AUROC=0.84; FIG. 6A) than changes in Spike expression (AUROC=0.81; FIG. 9A). Among measures of immune escape, the binding contributions of known antibody epitopes (antibody binding score; see materials and methods) to anti-SARS-COV-2 antibodies were most predictive of mutation spread (AUROC=0.75; FIG. 6A). Natural Language Processing (NLP) scores for sequence plausibility (grammaticality)¹⁸ were similarly predictive (AUROC-0.77; FIG. 6A) 18. However, CD4+ or CD8+ T-cell immunogenicity did not offer significant explanatory power for mutation spread (AUROC=0.59; FIG. 9A). The best evolutionary feature for prediction of spread (AUROC=0.91; FIG. 6A) was obtained from Fixed Effects Likelihood (FEL²¹) from the Hyphy package [http://www.hyphy.org]²² which tests for pervasive negative or positive selection across the internal branches of a phylogenetic tree. Positive selection occurs when new mutations are more fit than wild-type, leading to an increase in amino acid diversity over time, whereas negative selection eliminates non-wild-type amino acids at that site.

The highest predictive performance, however, was obtained from epidemiological features; i.e., variables which more directly take into account sampled mutation counts (Table 1). Although multiple epidemiology-based features were highly predictive, the most predictive variable in this feature category was “Epi Score”, the exponentially weighted mean ranking across the other epidemiological variables (AUROC=0.94). This metric captures both lineage expansion and recurrent mutation that occurs in multiple variant lineages by convergent evolution. Both of these aspects of spread indicate mutation fitness. The utility of recurrent mutation signals is consistent with recent findings that convergent evolution plays a significant role in SARS-COV-2 adaptation²³.

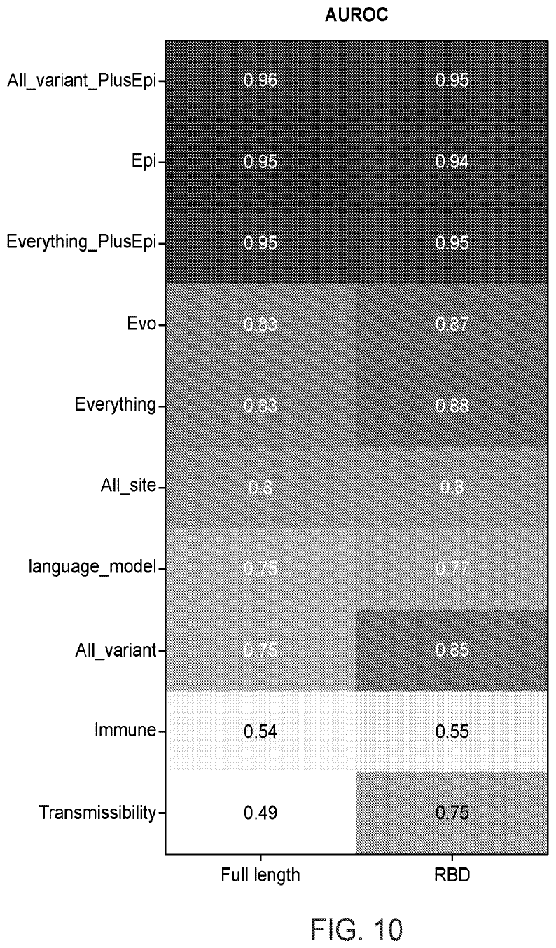

Outside of the RBD, there is less experimental annotation of amino acid mutations. CD4+ and CD8+ T-cell immunogenicity had little explanatory power across the full-length Spike sequence (max AUROC of 0.54). Language model grammaticality performance was slightly reduced in predictive impact compared to the same measure from RBD alone, with an AUROC of 0.73. As observed for the RBD alone, within Spike the best predictive performance were obtained with evolutionary (AUROC=0.84) and epidemiologic (AUROC=0.94) measures (FIG. 6A). The performance of other feature sets is presented in FIG. 10. Specifically, FIG. 10 shows the predictive performance of integrated feature sets at baseline. AUROC values within the RBD and across full-length Spike for a variety of feature sets tested. The exact variables included in each feature set can be found in Table 7.

Next, the robustness of this approach was interrogated in response to changing drivers of SARS-COV-2 evolution. For example, it is possible that selection due to immune pressure will increase with time as more individuals become immune through infection or vaccination. For example, the P.1 lineage is thought to have spread rapidly in Brazil largely due to immune selection in a population with high seroprevalence²⁴. Thus, the predictive performance of antibody binding scores was measured by taking as a proxy for B-cell immunodominance (Table 1)²⁵. Taking the maximum of this value across antibodies at a given site yields the maximum antibody binding score. The predictiveness of this metric increased from nearly uninformative early in the pandemic (p-value for difference from random=0.53), to an AUROC of 0.75 (p<le-4; FIG. 9C) for predicting spreading mutations during the third wave of the pandemic (FIG. 6B).

A similar analysis for all variables in both Spike and RBD is presented in FIGS. 9A and 9B. Throughout this transition from lower to higher levels of immune selection, epidemiological features maintain their performance, achieving an AUROC of 0.98 for the final evaluation period (FIG. 6B). A summary of performance for all features across time and within the RBD and across full-length Spike can be found in FIGS. 9A and 9B.²⁴

In summary, immunity, transmissibility, evolution, language model, and epidemiologic features all predicted mutation spread. The analyses suggested predictive performance of features related to immune escape increased significantly over time, which could be indicative of changing evolutionary pressures as rates of natural—and vaccine-derived immunity rise. These observations indicate that it is possible to predict which mutations may spread rapidly, and that the methodology for doing so can accommodate changes to the underlying selective forces over the course of the pandemic. Epidemiologic features in particular display superior accuracy and robustness over time.

### III. Assessment of Feature Sets Across the Pandemic

Methods further involve training a predictive model to predict spreading mutations using sets of features identified above. In one embodiment, a logistic regression model with baseline features as inputs was constructed. Within each feature set, the features used for prediction were selected using forward feature selection, cross-validated within each training dataset. To minimize correlation between training and test amino acid mutations through shared haplotype structure, model training was arranged so that mutations from the same phylogenetic clade were never split across the training and test datasets, thus minimizing information leakage.

The direct univariate ranking of amino acid mutations described in the previous section further validated that results were not due to overfitting based on correlations between training and test set observations. Since there was no model fitting for the univariate analysis, there could be no overfitting. For the third wave of infections at one month of anticipation (FIG. 4A), the best predictors were positive selection features (AUROC=0.83) and epidemiologic features

(AUROC-0.95; FIG. 6C). Immunity and transmission features did not improve predictive power of positive selection features (AUROC-0.83). No additional variables that improved upon the performance of epidemiological features were identified. The performance of the trained model was comparable to the performance of the Epidemiology Score (“Epi Score”) metric (FIG. 6A vs. FIG. 6C). Therefore, to simplify reproducibility and further minimize the risk of overfitting, the Epi Score was used to predict mutation spread going forward.

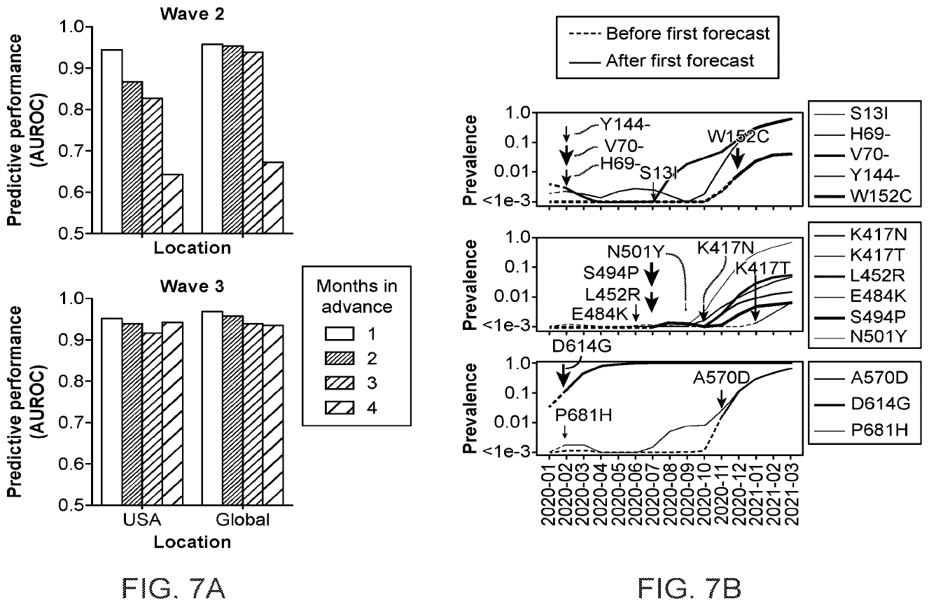

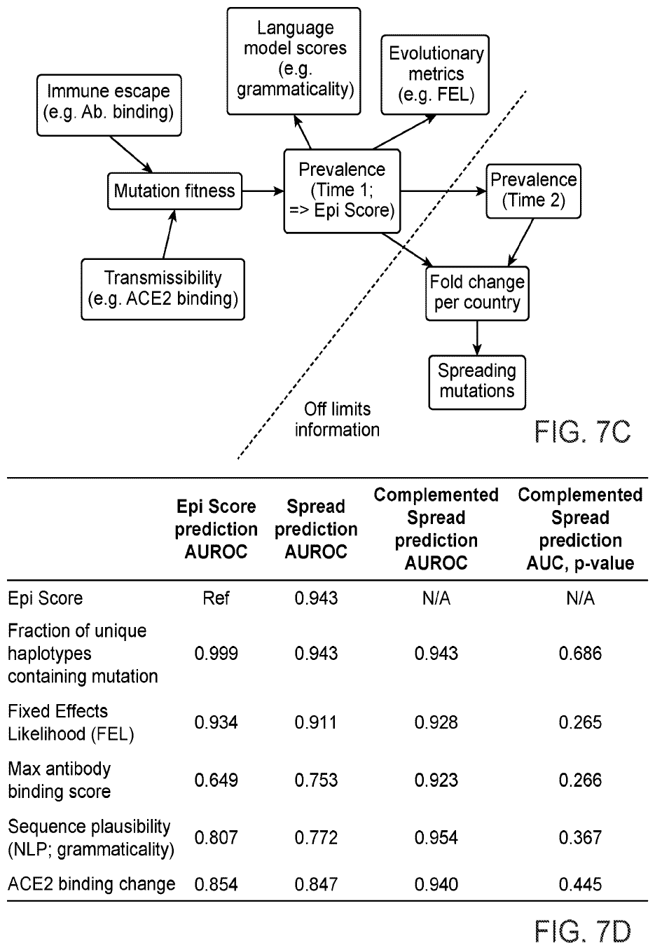

Next, the performance of the Epi Score was tested in its ability to forecast spreading mutations in both the second and third wave; with one, two, three, and four months of anticipation. Here, mutations can be predicted with an AUROC above 0.85 at least two months in advance, in both wave 2 and wave 3 both within the United States and globally. Prediction of mutations in Wave 2 was still better than random four months in advance, despite only having access to the fewer than 600 viral sequences that were available in January and February of 2020. In Wave 3, AUROCs of 0.87 was observed for predicting both United States and globally spreading mutations four months in advance (FIG. 7A).

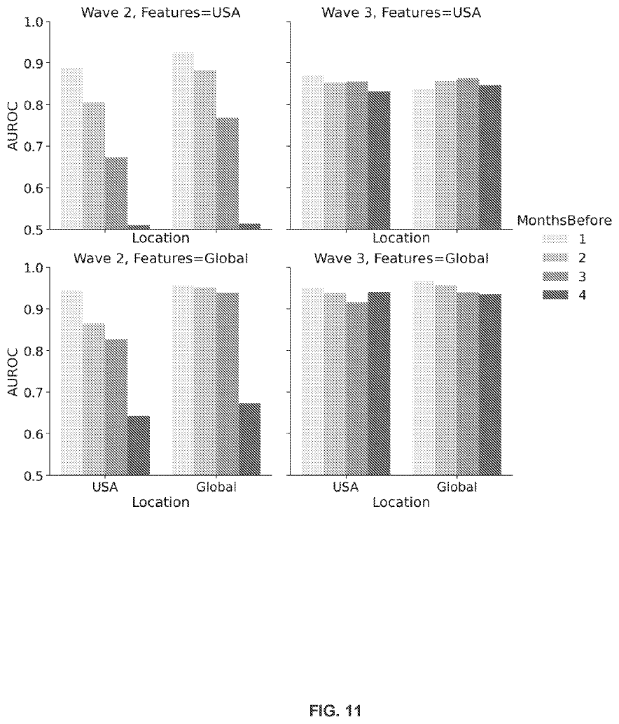

Specifically, FIGS. 7A-D show the early detection and causal mediation of spreading mutations. FIG. 7A shows the performance (AUROC) in predicting spreading mutations in the second and third waves of the epidemic (see FIG. 1C). Predictions are generated using a three-month window of data, preceding the wave by 1-4 months. FIG. 7B is a depiction of where in their growth trajectories VOC mutations were first forecast to spread. Dotted lines denote the part of the curve where the variant had not yet been forecast to spread. Solid lines denote the period after first forecast. A version of this plot with mutations grouped by lineage can be found in FIG. 11. To reduce overplotting, mutations are plotted in genomic order, but split into panels by genomic region: NTD, RBD, and other regions. FIG. 7C is an example model: viral fitness (determined by, e.g. changes to transmissibility and immune escape) drives viral prevalence at time 1 (as measured by global frequency, and geographic and haplotype distribution), which is captured by Epi score. Language model score or evolutionary metrics are summaries of GISAID data and therefore are shaped by mutation prevalence. Prevalence at time 1 predicts prevalence at time 2, which ultimately leads to mutation being defined as spreading. Therefore, prevalence at time 1 (as captured by Epi Score) mediate the effects of the biological variables that enhance viral fitness through transmissibility or escape adaptation. FIG. 7D shows performance of difference features. Specifically, to quantitatively test for mediation, variables were evaluated as to whether they were better at predicting top mutations that are in the top 200 Epi Score, compared to spreading mutations for time 2 versus time 1. Within the RBD, the major variables from each group generally predict Epi Score better than they predict spreading mutations. One exception was antibody binding score. In support of mediation, the analysis found little complementarity, as measured by the AUROC, when combining each variable with the most effective epidemiological variable.

Next, spreading mutations were analyzed to determine whether local (e.g., United States) or global dynamics drive mutation spread (FIG. 11). This examination was repeated for each analysis presented above. Global epidemiology metrics were best overall, and indeed, were generally more predictive of state-level mutation spread than the state-level metrics themselves. Specifically, FIG. 11 shows graphs predicting local and global spreading mutations in waves 2 and 3. Predictive performance (AUROC) for predicting mutations that are spreading i.) within the United States (“USA”, x-axis), and ii.) globally (“Global”, x-axis), as predicted by features calculated i.) in the United States (top row) and ii.) globally (bottom row). The analysis is repeated for Wave 2 (first column) and Wave 3 (second column), and for predictions 1, 2, 3, and 4 months in advance (colored bars).

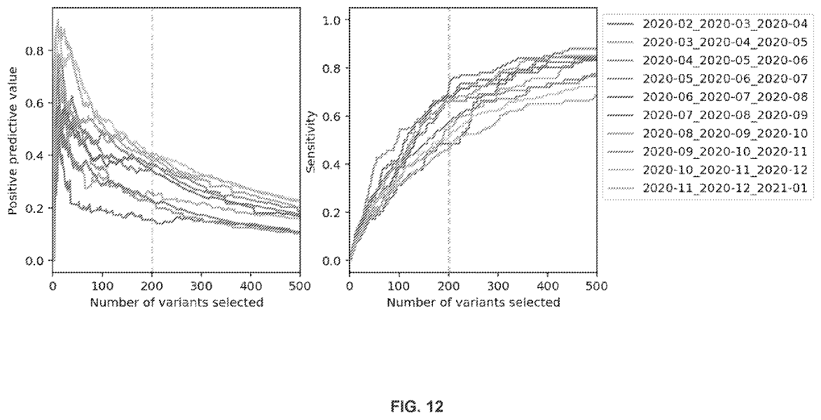

To illustrate the practical utility of Epi Score forecasting using global features, a sliding window analysis was performed to assess how early the spread of Spike mutations contained in current CDC VOCs can be forecasted. To be conservative, the date that a mutation was first forecast was considered as the earliest date at which it was predicted to spread in two subsequent analysis periods. For example, consider a mutation that was predicted to spread in June, October, and November. Since the mutation was not predicted to spread in July, the June date would be disregarded and the earliest forecasted date would instead be October. The chosen cutoff of the 200 top scoring mutations (FIG. 12) corresponded to a historical specificity between 93% and 97% across the sliding window period, with a specificity of 97% in the most recent period. Specifically, FIG. 12 shows graphs of model performance as a function of number of variants. To understand the tradeoff between the fraction of observed spreading mutations correctly forecast (sensitivity), and the fraction of predicted spreading mutations that are correct (positive predictive value), these quantities were examined as a function of the number of top-scoring mutations forecasted to spread. This analysis was repeated across time windows, denoted by the three months prior to the cutoff. 200 mutations was about the point at which increases in sensitivity began to level off, while maintaining a reasonable (>20%) positive predictive value.

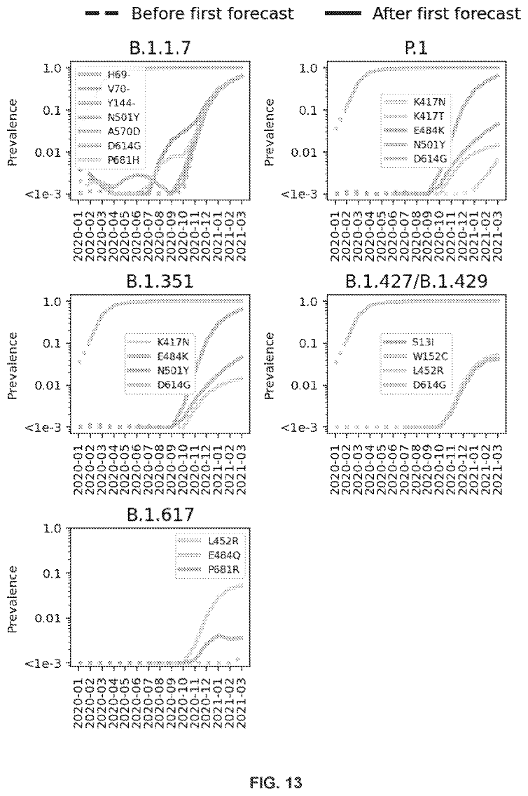

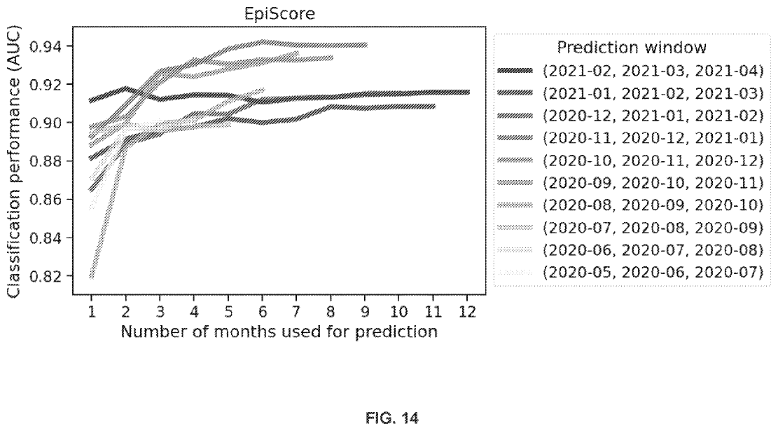

Next, a retrospective analysis was conducted which examined when the spread of current VOC Spike mutations could be predicted (FIG. 7B and FIG. 13). Specifically, FIG. 13 is a depiction of growth trajectories of various mutations and when they were first forecast to spread. Dotted lines denote the part of the curve where the variant had not yet been forecast to spread. Solid lines denote the period after first forecast.

Since D614G was already highly prevalent before sufficient data were available to inform predictions, early forecasting was not meaningful for this mutation. The rise of A570D was also rapid enough that it was not forecast until it reached 1.3% frequency. Even including these mutations, VOC mutations could be predicted an average of more than 5 months in advance of them reaching 1% global frequency. For the mutations forecasted prior to reaching this threshold, the average frequency at the time of first forecast was 0.16% (Table 4). This analysis was repeated for the more recently emerged B.1.617 strain first discovered in India. The L452R mutation from this strain was first forecast in July of 2020, while the P618R was first forecast in October of 2020. Its E484Q mutation was not forecast until March of 2021 (Table 5). Thus, this approach was robust enough to predict spreading mutations in two pandemic waves several months in advance. In particular, early warning of mutations in current VOCs and VOIs would have been possible before reaching worrisome levels of global spread.

### IV. Understanding Performance Through a Causal Lens

Seeking to understand the notably high performance of epidemiologic features, a directed acyclic graph was constructed to visualize the hypothesized causal relationships, and to probe whether relative trends in performance were consistent with the expectations that follow from this model (FIG. 7C). Epidemiologic features mediate the relationship between viral fitness and mutation spread. The rationale was that if a mutation's fitness were sufficient to drive it to appreciable prevalence at one time point (as measured by global frequency and geographic and haplotype distribution), it would likely drive it to higher prevalence in the future as well (unless it were outcompeted by a more fit adaptation, or the fitness landscape changes). This type of mediated relationship (fitness=>current prevalence=>future prevalence) implies that epidemiological prevalence features will capture information from both known and unknown drivers of selection. Their utility extends beyond measuring fitness, too. Even for two adaptive mutations of the same fitness, the one that is currently more prevalent has a higher likelihood of spreading further, since higher prevalence increases the influence of selection relative to genetic drift. This line of reasoning predicts that epidemiologic variables that capture initial prevalence, will provide a robust measure of demonstrated real-world fitness. Epistasis, public policy, and a range of other factors could obscure this relationship. However, by focusing on amino acid mutations across viral variants and geographic locations, these effects could be averaged out.

If this hypothesized model were reasonable, the expected result is that variables whose causal effects are mediated, as defined above, should predict epidemiologic variables at a comparable or even greater accuracy compared to spreading mutations. This is illustrated by comparing the first and second columns of FIG. 7D. With the exception of the maximal antibody binding score, all top variables predict Epi Score better than they predict mutation spread. The lower predictiveness of maximal antibody binding score for Epi Score would be consistent with a slight time lag effect due to shifting evolutionary pressures.

A second criterion for mediation is that information from these variables should not significantly complement the predictiveness of the epidemiologic variables alone. This is assessed by comparing the AUROCs of two-variable models in column 3 of FIG. 7D with the AUROC for Epi Score alone (0.94). The only nominal AUROC increase for a complemented model was observed for NLP sequence plausibility (0.95). This improvement was not statistically significant (p=0.367). Similarly, no other variable was found to exhibit statistically significant complementarity with Epi Score, either within the RBD or across full length Spike (see supplemental section “Mediation Analysis”, Table 6).

This examination of mediated causal relationships begins by assuming a causal graph based on prior knowledge. Such an approach is common to many causal inference methods²⁶ and represents a well-known limitation²⁶. Therefore, this analysis is a tool for more systematically considering the plausibility of the results. While it is generally difficult to verify the structure of proposed causal graphs, the findings described herein support that epidemiological variables mediate the effects of other classes of explanatory variables, and this may explain their high predictive accuracy.²⁶

### V. Forecasting Spreading Mutations

Encouraged by accurate prediction of spreading mutations in the second and third wave, the stability of performance in the face of changing selective dynamics, and the explainability of high predictive performance of epidemiologic features, the next step of analysis involved leveraging Epi Score on the current data to forecast mutations that may contribute to VOIs and VOCs over the coming months. Since global metrics outperformed metrics restricted to the United States, even for forecasting within the United States, global forecasting was used. Although shortening the feature calculation window to further mitigate the effects of shifting evolutionary dynamics was considered, longer feature calculation windows robustly improved performance across all prediction windows (FIG. 14). FIG. 14 shows the effect of the length of the feature calculation window on predictive performance. Colors indicate different time periods in which spreading mutations are predicted. The x-axis shows the number of months prior to this period used for feature calculation, and the y axis presents the classification performance (AUROC).

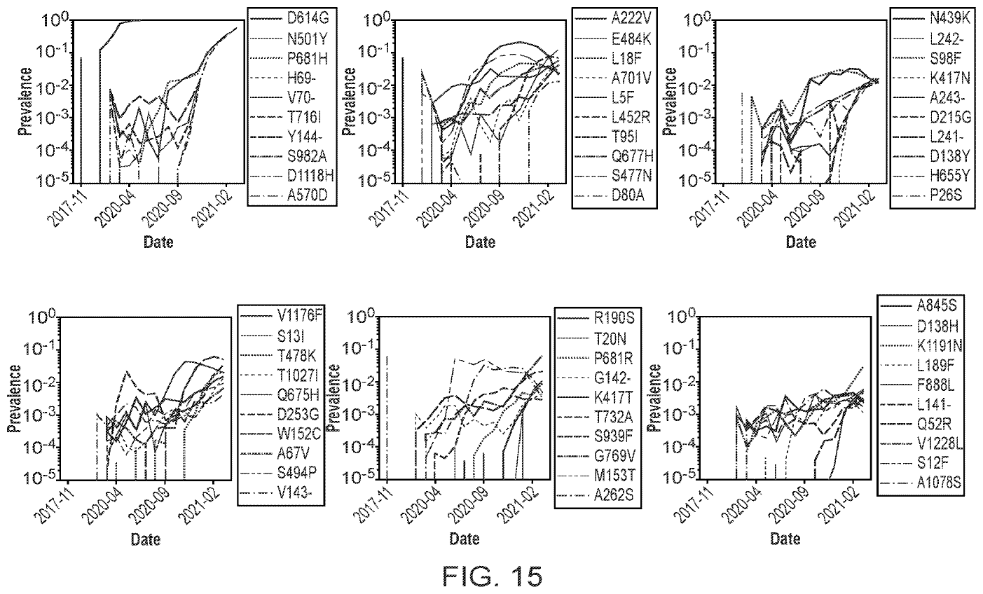

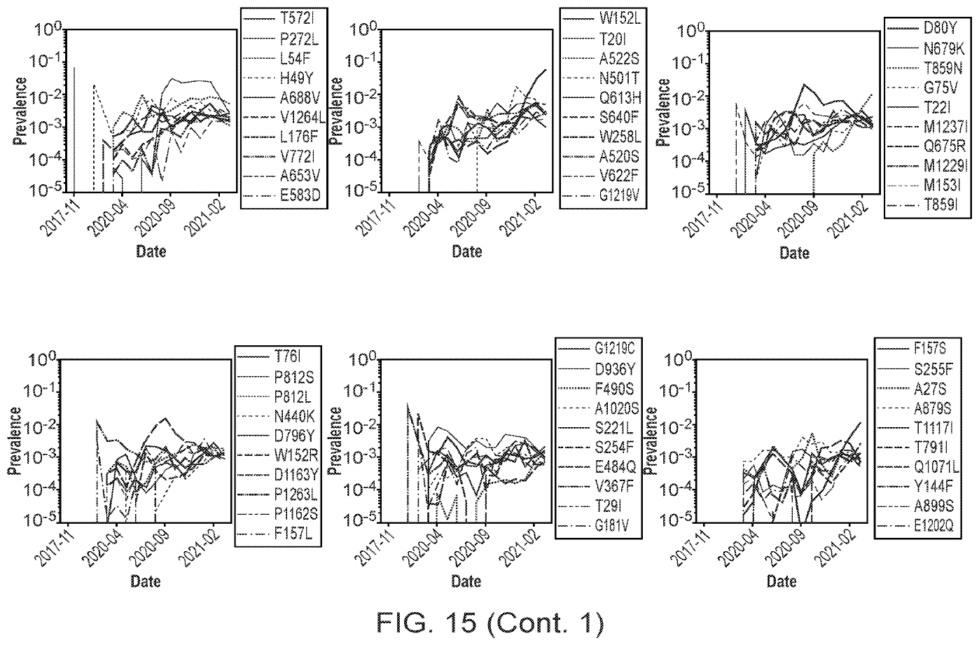

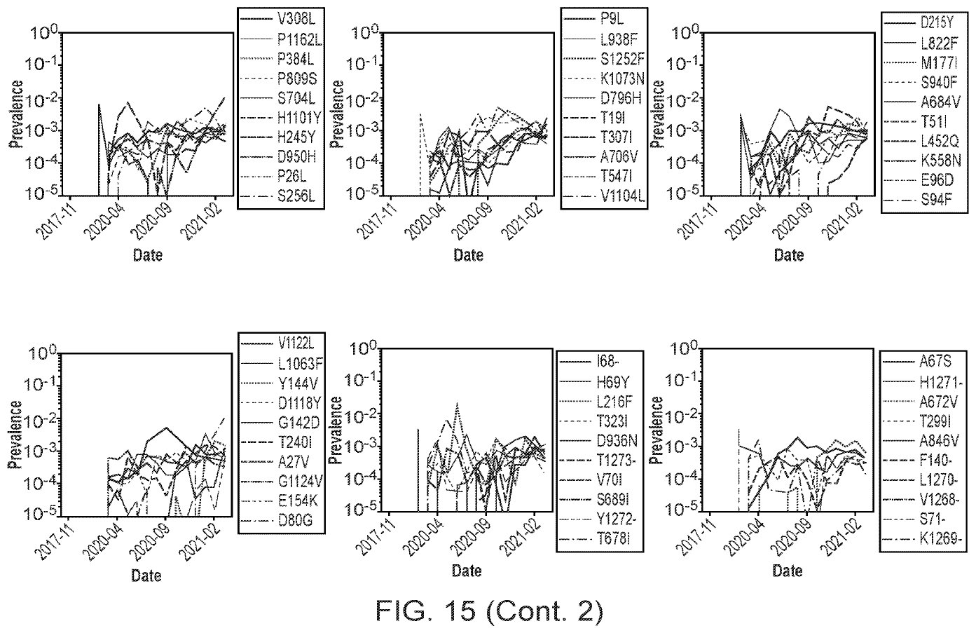

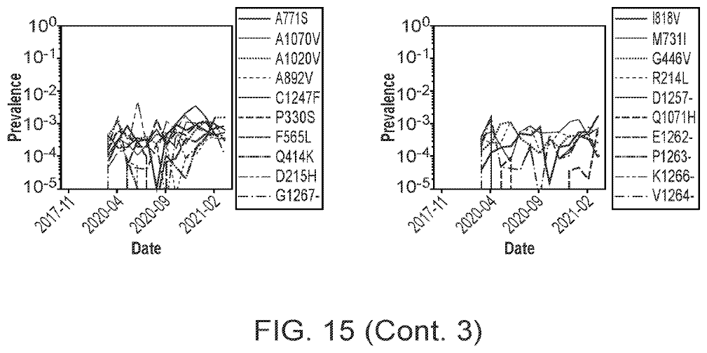

Table 2 shows a subset of predicted mutations that do not belong to the canonical UK B.1.1.7, Brazil P.1, South Africa B.1.351, or California B.1.427/B.1.429 VOC haplotypes, and obtain an Epi Score of at least 9.8 out of 10. A visualization of how the frequency of all forecast mutation have changed over time can be found in FIG. 15. Most forecast amino acid mutations have demonstrated consistent increases in global frequency. FIG. 15 shows population size-adjusted mutation frequencies for mutations forecasted to spread. Since global mutation frequencies are heavily biased by more intensive sequencing in a handful of countries, per-country mutation frequencies were re-weighted so they contributed to the total estimate according to population size rather than number of sequences generated. Panels show global frequencies over time.

Of the 22 highlighted mutations in Table 2, three occurred in the RBD, where high density experimental data are available. Of these, S477N scored in the 97th percentile for increased ACE2 binding among the 1022 observed RBM mutations. It also scored in the 91^{st} percentile for increased Spike expression and escape from therapeutic antibodies. T478K scored in the 92^{nd} percentile for increased ACE binding, while S494P scored in the 88th percentile for this attribute.

Finally, as an application of the forecasting analysis, forecasted mutations were analyzed for their intersection with the binding sites of clinical antibodies. A wide variation in the number of forecasted mutations per antibody epitope (Table 3) was found, ranging from 8 mutations for Celltrillion's CT-P59, to zero mutations for Vir's S309, which was designed to be robust to viral evolution by targeting a region that is conserved across coronaviruses²⁷. Outside of the RBD, a significant proportion of the forecasted mutations (39%) occur in the signal peptide and N-terminal domain (NTD), despite comprising 23% of the Spike sequence. This region is a focus of attention²⁸, as it is known to be subject of considerable immune and selective pressures¹.

In summary, methods disclosed herein are useful for forecasting spreading mutations and for forecasting future contributors to putative VOCs/VOIs. These predictions are consistent with in vitro data where it is available. A subset of forecast mutations could have implications for the continued efficacy of clinical antibodies, but that the level of these concerns varies widely.

### VI. Discussion

This work started with a working definition of a spreading amino acid mutations and leveraged this definition to deliver a systematic analysis of amino acid features predictive of mutation spread. Immunity, transmissibility, viral evolution, language models, and epidemiology features are all predictive of spreading mutations. This modeling framework was further employed to show that immune escape has played a greater role in SARS-COV-2 evolution over time over time.

Epidemiological features (in particular, the proposed “Epi Score” metric) are most sensitive and specific for the prediction of mutations with potential to spread both globally and within the United States. This yielded a simple, explainable, and highly accurate model for forecasting mutations several months in advance, across multiple pandemic waves. This model uses genomic surveillance data (which is sufficient for achieving high model performance). Confidence in the prediction of spreading mutations comes through retrospectively evaluating predictions across multiple waves of the pandemic and verifying consistency with a plausible causal framework. Further, long observed lags between the earliest warning signals and high population frequency of current VOC and VOI mutations gives support for using forecasting to anticipate the spread of future concerning mutations.

Furthermore, a forecast of the amino acid mutations that are most likely to spread over the coming months are provided. These amino acid mutations can differentially impact clinical antibodies. These results provide a foundation for future improvement. For example, progress in the representativeness of population sequencing efforts, early and complete mapping of epitopes, and broader coverage of site directed mutagenesis and downstream functional readouts may improve the performance of this and future predictive models. Although these results have demonstrated Epi Score is robust to the shifts observed in evolutionary pressures during the pandemic so far; performance can be monitored in real-time, and if necessary, re-tuned to capture novel behavior. This approach can also be generalized and improved upon to stay ahead of evolutionary cycles in other pathogens, when sufficiently rich and representative genomic sampling is available.

### VII. Methods

Variable definitions and data sources. The definitions of the variables presented, how they are grouped into categories, and where they can be retrieved, can be found in Table 7.

Code availability and environment. Analyses on GISAID data extracts were conducted in python (Python Software Foundation. Python Language Reference, version 3.7. Available at http://www.python.org). Code was built in Jupyter lab notebooks²⁹ and relied upon a number of common data analysis libraries³⁰⁻³³

Sequence access and alignment. The viral sequences and metadata were obtained from GISAID EpiCoV project (https://www.gisaid.org/). Analysis was performed on sequences submitted to GISAID up to Apr. 24, 2021. The spike protein sequences were either obtained directly from the protein dump provided by GISAID or, for the latest submitted sequences that were not incorporated yet in the protein dump at the day of data retrieval, from the genomic sequences, with Exonerate³⁴ 2.4.0-haf93ef1_3 (https://quay.io/repository/biocontainers/exonerate?tab-tags) using protein to DNA alignment with parameters-m protein2dna--refine full--minintron 999999--percent 20 and using accession YP_009724390.1 as a reference.

Multiple sequence alignment of all human spike proteins was performed with mafft35 7.475--h516909a_0 (https://quay.io/repository/biocontainers/mafft?tab-tags) with parameters-auto--mapout--reorder--keeplength--addfragments using the same reference as above. Spike sequences that contained>10% ambiguous amino acid or that were less than 80% of the canonical protein length were discarded.

A total of 1,104,875 sequences were used for analysis. Mutations were then extracted as compared to the reference with R 4.0.2 (https://www.r-project.org/) using Biostrings 2.56.0 (https://bioconductor.org/packages/Biostrings) and haplotypes were obtained by combining all amino acid mutations (substitutions, insertions, and deletions) identified on the Spike protein when compared to the reference sequence. GISAID reports viral consensus sequences for individuals. Thus, for this analysis the presence of a single virus in a single sample was assumed. This approach will not detect evolution of viral quasi-species within individuals but allows for the characterization of dominant spreading mutations in populations³³.

Defining spreading mutations. As described in the main text, mutations were selected based on a Fisher's exact test for frequency fold change per country, adjusted for multiple comparisons. This approach was selected after exploratory analysis found that more granular trend tests such as the Mann-Kendall trend test were less favorably powered in low-data countries. Comparisons were adjusted using the function statsmodels.stats.multitest.fdrcorrection from the statsmodels package³² with an alpha of 0.05. This applies a Benjamini-Hochberg correction. 2×2 tables were constructed and used for the Fisher's exact test in the following manner. Within each country, four counts were tabulated: the number of sequences containing the mutation of interest, versus those that did not; in one window before, and one after the date cutoff (e.g. Nov. 1^{st}). From each table, a fold change and an associated comparison-adjusted p-value were calculated. Mutations with a significant p-value from any country were accepted. The number of comparisons for the adjustment was taken as the number of countries times the number of observed mutations worldwide.

Analyzed features. B cell epitopes and CD4+ and CD8+ T epitopes in the viral Spike protein^{16,36} were investigated as features that might predict spreading mutations. Additionally integrated were in vitro mutagenesis data quantifying ACE2 binding of the viral Spike protein, expression of the viral spike protein, and escape from monoclonal antibody neutralization as measured in pseudovirus assays and/or binding of monoclonal antibodies to the Spike protein^{14,20}. In addition, features included viral genome conservation such as RNA secondary structure constraint²² and conservation of amino acids, as quantified by Shannon entropy, across the three sarbecovirus clades that encompass both SARS and SARS-COV-2. Additional metrics of positive selection via MEME and FEL²³ were assessed. The variation in the viral proteome as captured by novel natural language learning tools¹⁹ were analyzed. Epidemiologic features were calculated from the training periods, such as mutation frequency, and the distribution of mutations across countries and viral variant backgrounds. Additionally, an integrated epidemiology score (“Epi Score”) was calculated as the exponentially weighted mean ranking across mutation frequency, the fraction of unique variant sequences that contain an amino acid mutation, and the number of countries in which a mutation was been observed. Briefly, to calculate Epi score, the percentile of each component score (p) was calculated, and from this calculated a new score (10**p). The average of these scores between the metric pair resulted in the combined score that ranged between 1 and 10.

Preparing feature sets. Deep mutational scan data^{20,37}, were retrieved from the following repository: https://github.com/brianhie/viral-mutation. For T-cell data where scores are associated to oligonucleotides instead of mutations or sites, overlapping scores were averaged per site. When there were multiple experimental conditions, the maximum value per site was taken.

Antibody binding energies were calculated using Molecular modeling software MOE²⁵ (v2019.0102). To produce the antibody binding score, the first step involved calculating pairwise binding energies (the sum of van der Waals, ionic, aromatic, and hydrogen-bond interactions) between each residue in the antigen epitope and each residue in the corresponding antibody Fab paratope, including all residues within a cutoff distance of 5.0 Å from the epitope/paratope interface. All structures were prepared prior to these calculations using the structure preparation, protonation and energy minimization steps in MOE, with default settings. The binding energies of each epitope residue that interacted with multiple Fab residues were added together and the percentage of the binding energy contributed by each epitope residue to the total binding energy was calculated. When more than one copy of the complex was present in the asymmetric unit, binding energy contributions were averaged across all copies. An overall binding energy per site was calculated as the max score across all antibodies.

Interspecies conservation was calculated from a nucleotide multiple sequence alignment of 44 sarbecoviruses. The Shannon entropy was calculated for each column in the alignment. Non-ATGC letters (including gaps) were ignored. RNA structure SHAPE-seq intensities are downloaded from: http://incarnatolab.com/downloads/datasets/SARS Manfredonia 2020/XML tar gz)³⁸. They are post processed by taking the mean of each 12-nucleotide sliding window and the window centered on a given nucleotide is used.

Natural language processing (NLP) neural network features involve the grammaticality and semantic change scores reported by Hie et al.¹⁸ in which a bidirectional long short-term memory (BiLSTM) model was trained on Spike sequences from GISAID and GenBank. Two versions of the model were obtained: the original model trained on sequences through sampled prior to Jun. 1, 2020, and a second model that was retrained starting from random weight initializations on GISAID Spike sequences sampled prior to Nov. 1, 2020. For all prediction periods after November 1^{st}, the latter model was used. For prediction periods before this time, the former model was employed.

Natural selection features were generated using MEME³⁹ and FEL²¹ methods implemented in the HyPhy package²² (version 2.5.31). Data preparation, alignment, and tree inference were performed using an existing pipeline (https://github.com/veg/SARS-COV-2/tree/compact/). Briefly, the pipeline curates sequences to remove low quality genomes and filter out potential sequencing errors and compresses the input to unique haplotypes over each gene region. A codon-aware mapping and multiple sequence alignment of gene regions is followed by rapid phylogenetic tree inference, and site-level selection analyses applied to internal tree branches (a standard procedure for viral intra-species data). FEL tests for pervasive negative or positive selection, while MEME tests for episodic positive selection. Both tests report p-values (based on the likelihood ratio tests); MEME further reports the number of branches which provide support for the positive selection model component.

For epidemiologic variables, the “fraction of unique haplotypes” metric was defined as the proportion of the known haplotype backgrounds in which a given mutation occurred. The “mutation frequency” metric is defined as the fraction of sequenced individuals who had a mutation at that site. The “number of countries” metric is defined as the number of countries in which a mutation is observed in at least two sequences.

Epi Score was calculated as an exponentially weighted mean of the mutation ranks according to mutation frequency, fraction of unique haplotypes in which the mutation occurs, and the number of countries in which it occurs. Specifically, this involved (i) calculating the percentile for each score, for each metric, (ii) exponentiating percentile to the power of 10, and (iii) averaging these exponentiated percentiles. The effect of this procedure is to assign highly differentiated weights to high rankings, and relatively small and similar weights to mutations that are not at the top of the list. For example, top ranked mutation versus a 90^{th} percentile mutation will have a score difference of 2.1 (10 vs 7.9), whereas a mutation at the 50^{th} percentile and one at the 40^{th} percentile will have a score difference of 0.65 (3.16 vs 2.51). This scheme is particularly advantageous if measurements for lower-ranked entities are more noisy than higher ranked ones, and/or if one wants to up-weight high rankings.

For conversion from mutation-to site-level scores, the site level score was taken to be the maximum of mutation scores at that position. For the conversion of site-to mutation-level scores, the site-level score was assigned to all observed mutation at that position. In cases where data needed to be imputed, min-imputation was performed. For example, all sites without measured antibody binding energies were assigned a binding energy of zero. For all metrics, in cases of multiple experimental conditions, the max score per site or mutation (as appropriate) was taken. This was appropriate because the few metrics where lower scores implied a higher probability of spread (e.g. MEME p-values) did not have missing values.

Quantifying predictive performance. Predictive performance was quantified using the area under the receiver operator characteristic curve (AUROC). This quantity can be interpreted as the probability that a given score correctly ranks a random pair of positive and negative examples. Performance was assessed by two methods: (i) direct univariate ranking and (ii) model fitting with sets of features. The AUROC for univariate ranking was calculated as the maximum AUROC upon sorting by that metric in either ascending or descending order. Receiver Operator Characteristic (ROC) curves were generated by varying the numerical cutoff ‘C’ on each metric beyond which a mutation is called to be spreading. Given these calls, sensitivity and specificity values were calculated for each value of C tested. Plotting sensitivity versus specificity yields the ROC curve. The area under the ROC (AUROC) was then used to quantify the capacity for that variable to distinguish spreading from non-spreading amino acid mutations.

For model fitting, performance was assessed by cross-validation. This involves partitioning the data into chunks or “folds” and iteratively predicting each (test) fold based on training with all the other chunks. In this procedure, it is important to make sure that correlated observations are kept in the same fold so that information does not leak between the training folds and the test fold. As a hypothetical example, a model could memorize the attributes of one identical twin in a training set to predict the values of the other in the test set. In the case of mutations, the co-occurrence of mutations on the same haplotypes could introduce a correlation in their metrics. To mitigate this issue, mutations from the same clade were always included in the same fold. Clades were defined according to GISAID annotation. The following clades were used to define folds: G, GH, GR, GRY. The remaining smaller clades were pooled into a single fold. This resulted in five folds ranging in size from around 700 to 2000 mutations. AUROC values were then calculated within each test fold and averaged across test folds to yield an overall performance.

Predictive performance of sets of features. Prediction was performed using forward feature selection followed by logistic regression. The criterion for forward selection was cross-validated AUROC of the logistic regression model within the training set. Feature selection and model fitting were performed separately within each fold of the outer cross validation loop. Logistic regression was chosen due to its sample efficiency. Random forest classifiers obtained worse performance. Combined models did worse than individual features if there was no feature selection step. A select K best feature selection strategy was also employed, which generally recapitulated the performance of the single best feature. These results suggest that limited sample size amplifies the effect of noisy features, and that greedily selecting for high AUROC features does not do a good job of selecting for complementarity. The members of each of the feature sets are enumerated in Table 7.

Selected features. Since a different model is fit for each cross-validation fold, a single model was retrained on all data to produce a single set of selected features for each feature set. Mediation analysis. The strength of predictions based solely on the epidemiological

features led the study to consider a hypothesized causal model (FIG. 7C) to explain the effectiveness of these features relative to the contribution of biological measurements. The biological factors determine viral fitness, which in turn drives spread, as measured via epidemiology. As illustrated in (FIG. 7C), epidemiology and evolution-based measures both draw on empirical variation, as captured by GISAID. Epidemiologic variables likely demonstrate superior performance because they are most proximal to the outcome variable, and therefore mediate the effects of the other variables. In causal inference, a mediated variable is a quantity that indirectly contributes to an outcome of interest (in this case spreading mutations) by altering an intermediate factor (a mediator; e.g., initial mutation spread). The classical Baron and Kenny test for mediation can be divided into three steps (i) make sure the variable of interest predicts the outcome, (ii) verify that the variable of interest predicts the mediator, and (iii) show that the variable of interest does not add to the predictive performance of the mediator when including both in a single model.

Step 1 was performed as part of the baseline analysis, and the complete results of this can be found in FIGS. 9A and 9B. For step 2, since few variables showed above-random performance outside of the RBD, analysis was focused on the RBD to predict the putative mediator (Epi Score). This surrogate outcome was predicted by first binarizing it to indicate whether the mutation score was in the top N mutations, where N is two times the number of mutations that spread in the observed dataset. This was multiplied by two after consulting the positive predictive values in FIG. 12. These results, shown by comparing the first and second columns in FIG. 7C, demonstrate that variables that are predictive of spread are also predictive of the epidemiologic predictor, with approximately the same magnitude. Therefore, criteria 1 & 2 have been fulfilled.

Finally, predictive models with each variable were fit in addition to the epidemiologic predictor to test for complementarity. Supervised models trained on full length spike tended to perform poorly with variables that are only observed within the RBD. Specifically, supervised models significantly decreased in performance when including these variables, indicating overfitting. To address this issue, and to make the results more comparable to the univariate analysis, a single score was generated from the variable pairs by exponentially weighting the ranks of each metric. This was performed according to the same procedure as the Epi Score. Specifically, the percentile of each score (p) was calculated, and from this a new score (10**p) was calculated. The average of these scores between the metric pair resulted in the combined score.

Testing integrated predictive models across waves and time lags. For testing predictive models across different waves and time lags, below are the time periods that were used for wave 2. The first group denotes the feature calculation window, and the second group of dates in each set denote the time window in which variant growth was assessed.
[(“2020-01”, “2020-02”), (“2020-06”, “2020-07”, “2020-08”)],
[(“2020-01”, “2020-02”, “2020-03”), (“2020-06”, “2020-07”, “2020-08”)],
[(“2020-02”, “2020-03”, “2020-04”), (“2020-06”, “2020-07”, “2020-08”)],
[(“2020-03”, “2020-04”, “2020-05”), (“2020-06”, “2020-07”, “2020-08”)]

Below are the time periods used for wave 3.
[(“2020-05”, “2020-06”, “2020-07”), (“2020-11”, “2020-12”, “2021-01”)],
[(“2020-06”, “2020-07”, “2020-08”), (“2020-11”, “2020-12”, “2021-01”)],
[(“2020-07”, “2020-08”, “2020-09”), (“2020-11”, “2020-12”, “2021-01”)],
[(“2020-08”, “2020-09”, “2020-10”), (“2020-11”, “2020-12”, “2021-01”)]

Forecasting spreading mutations. The list of forecast mutations was generated by calculating Epi Score on the most recent three months of data and taking the top 200 ranked mutations. The threshold of 200 mutations was chosen based on the analysis presented in FIG. 12.

Definition of Variants of Concern. Variants of concern were defined as those specified by the CDC, plus additional mutations which occurred at a rate of 80% of the most prevalent variant in the lineage3:
B.1.1.7 (CDC VOCs): H69-, V70-, Y144-, N501Y, A570D, D614G, P681H, T716I, S982A, D1118H
B.1.351 (CDC VOCs): K417N, E484K, N501Y, D614G
B.1.351 (additional associated mutations: D80A, D215G, L241-, L242-, A243-, A701V
P.1 (CDC VOCs): K417T, E484K, N501Y, D614G
P.1 (additional associated mutations): L18F, T20N, P26S, D138Y, R190S, H655Y, T1027I, V1176FB.1.427 (CDC VOCs): L452R, D614G
B.1.427 (additional associated mutations): S13I, W152C B.1.429 (CDC VOCs): S13I, W152C, L452R, D614G

### Example 2: Predicting Spread of SARS-COV-2 Pathogens including Delta and Omicron Variants

In this Example, various mutations of SARS-COV-2 were retrospectively analyzed to predict likelihood of spread and then compared to the actual prevalence of the various mutations. In particular, various mutations of SARS-COV-2 variants (e.g., Alpha, Beta, Delta, and Omicron) were analyzed to predict likelihood of their spread. This was of particular interest given the recent spread of both the Delta and Omicron variants in late 2021 and early 2022. In particular, mutations of the Delta variant included AY.20, AY.33, AY.127, and AY98.1. Mutations of the Omicron variant include BA.2, B.2.12, BA.2.12.1, and BA.5.

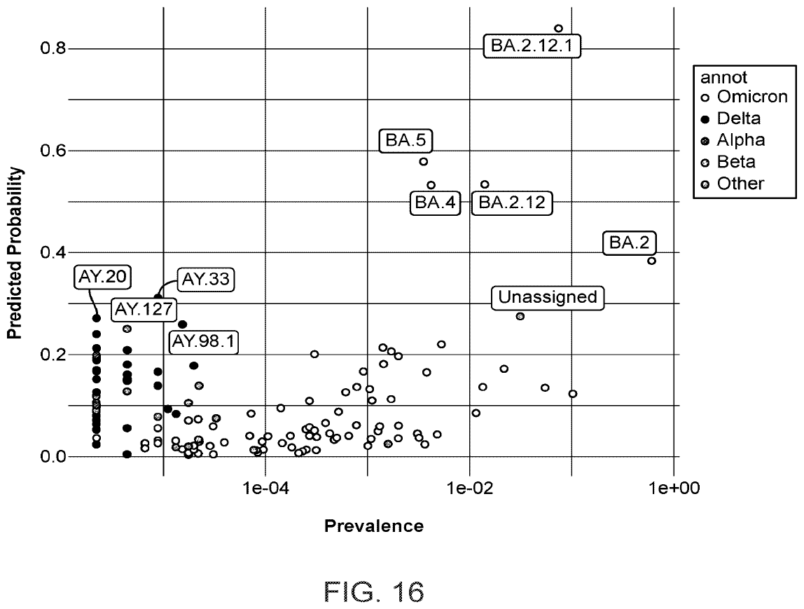

FIG. 16 shows the relationship between predicted probability of spread and actual prevalence of certain mutations. For example, a number of Delta variants (e.g., AY.20, AY.33, AY.98.1, and AY.127) as well as Omicron variants (e.g., BA.2, BA.2.12, BA.2.12.1, BA.4, BA.5) were predicted to spread, which aligns with their actual high prevalence. In particular, the predictive model correctly predicted spread of the AY.33 Delta variant, which became prevalent in the United States in Q4 2021. Similarly, the predictive model correctly predicted spread of the BA.2 Omicron variant, as well as the BA.2.12 and BA.2.12.1 subvariants (characterized by L452Q and S: S704F mutations), which were prevalent in both the US and the world in Q1 2022 (and continue to be highly prevalent to date).

Finally, the predictive model predicted high likelihoods of the spread of both the BA.4 and BA.5 omicron variants (characterized by L452R and F486V mutations). These variants were identified in January 2022 in South Africa, and to date (e.g., June 2022), the BA.4 and B.5 variants have not yet become the dominant omicron variant in either the US or across the world. However, based on the predicted likelihoods, BA.4 and BA.5 may become the more dominant variant in the future.

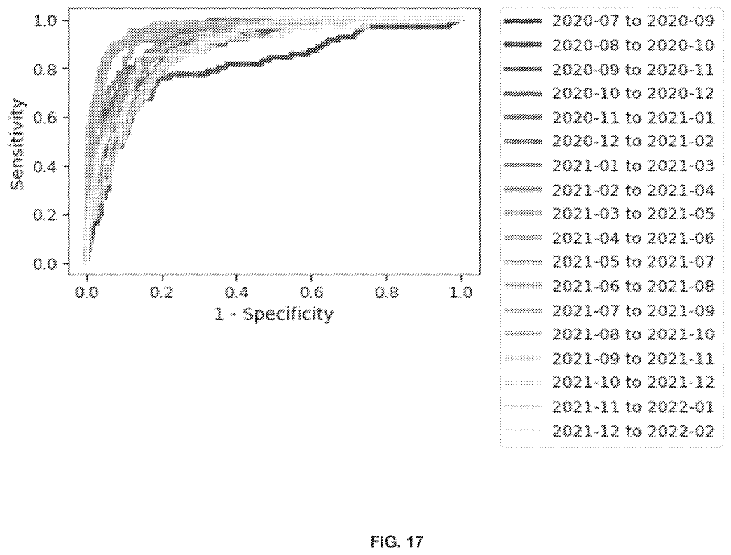

FIG. 17 shows predictive performance of the model over 18 sliding window periods. Specifically, each window period represents a 3 month period of time across the time frame between July 2020 to February 2022. Generally, as shown in FIG. 17, the performance of the predictive model, as characterized by the area under the curve (AUC) values, increased over successive window periods. For example, the predictive model exhibited higher performance over the most recent 3 month window period (e.g., December 2021 to February 2022) in comparison to the earlier 3 month window periods (e.g., July 2020 to September 2020). This indicates that over time and with additional training data, the predictive model is able to more accurately predict mutations that are likely to lead to pathogenic spread in the future.

### REFERENCES

1. McCallum, M. et al. N-terminal domain antigenic mapping reveals a site of vulnerability for SARS-COV-2. Cell (2021) doi: 10.1016/j.cell.2021.03.028.
2. Elbe, S. & Buckland-Merrett, G. Data, disease and diplomacy: GISAID's innovative contribution to global health. Global Challenges 1, 33-46 (2017).
3. Control, C. for D. SARS-COV-2 Variants of Concern. https://www.cdc.gov/coronavirus/2019-ncov/cases-updates/variant-surveillance/variant-info.html (n.d.).
4. Adiga, A. et al. All Models Are Useful: Bayesian Ensembling for Robust High Resolution COVID-19 Forecasting. Medrxiv 2021.03.12.21253495 (2021) doi: 10.1101/2021.03.12.21253495
5. Zhao, H. et al. COVID-19: Short term prediction model using daily incidence data. Plos One 16, e0250110 (2021).
6. Ray, E. L. et al. Ensemble Forecasts of Coronavirus Disease 2019 (COVID-19) in the U.S. Medrxiv 2020.08.19.20177493 (2020) doi: 10.1101/2020.08.19.20177493.
7. Control, C. for D. COVID-19 Forecasts: Cases. https://www.cdc.gov/coronavirus/2019-ncov/cases-updates/forecasts-cases.html (n.d.).
8. Padane, A. et al. First detection of the British variant of SARS-COV-2 in Senegal. New Microbes New Infect 100877 (2021) doi: 10.1016/j.nmni.2021.100877.
9. Valesano, A. L. et al. Temporal dynamics of SARS-COV-2 mutation accumulation within and across infected hosts. Plos Pathog 17, e1009499 (2021).
10. Charkiewicz, R. et al. The first SARS-COV-2 genetic variants of concern (VOC) in Poland: The concept of a comprehensive approach to monitoring and surveillance of emerging variants. Adv Med Sci 66, 237-245 (2021).
11. Dejnirattisai, W. et al. Antibody evasion by the P.1 strain of SARS-COV-2. Cell (2021) doi: 10.1016/j.cell.2021.03.055.
12. Collier, D. A. et al. Sensitivity of SARS-COV-2 B.1.1.7 to mRNA vaccine-elicited antibodies. Nature 1-10 (2021) doi: 10.1038/s41586-021-03412-7.
13. Starr, T. N., Greaney, A. J., Dingens, A. S. & Bloom, J. D. Complete map of SARS-COV-2 RBD mutations that escape the monoclonal antibody LY-CoV555 and its cocktail with LY-CoV016. Cell Reports Medicine 100255 (2021) doi: 10.1016/j.xcrm.2021.100255.
14. Starr, T. N. et al. Deep mutational scanning of SARS-COV-2 receptor binding domain reveals constraints on folding and ACE2 binding. Cell (2020) doi: 10.1016/j.cell.2020.08.012.
15. Starr, T. N. et al. Prospective mapping of viral mutations that escape antibodies used to treat COVID-19. Science 371, 850-854 (2021).
16. Agerer, B. et al. SARS-COV-2 mutations in MHC-I-restricted epitopes evade CD8+ T cell responses. Sci Immunol 6, eabg6461 (2021).
17. Tarke, A. et al. Negligible impact of SARS-COV-2 variants on CD4+ and CD8+ T cell reactivity in COVID-19 exposed donors and vaccinees. Biorxiv 2021.02.27.433180 (2021) doi: 10.1101/2021.02.27.433180.
18. Hie, B., Zhong, E. D., Berger, B. & Bryson, B. Learning the language of viral evolution and escape. Science 371, 284-288 (2021).
19. Hoffmann, M., Kleine-Weber, H. & Pohlmann, S. A Multibasic Cleavage Site in the Spike Protein of SARS-COV-2 Is Essential for Infection of Human Lung Cells. Mol Cell 78, 779-784.e5 (2020).
20. Greaney, A. J. et al. Complete Mapping of Mutations to the SARS-COV-2 Spike Receptor-Binding Domain that Escape Antibody Recognition. Cell Host Microbe 29, 44-57.e9 (2021).
21. Pond, S. L. K. & Frost, S. D. W. Not So Different After All: A Comparison of Methods for Detecting Amino Acid Sites Under Selection. Mol Biol Evol 22, 1208-1222 (2005).
22. Pond, S. L. K. et al. HyPhy 2.5-A Customizable Platform for Evolutionary Hypothesis Testing Using Phylogenies. Mol Biol Evol 37, 295-299 (2019).
23. Martin, D. P. et al. The emergence and ongoing convergent evolution of the N501Y lineages coincides with a major global shift in the SARS-COV-2 selective landscape. Medrxiv 2021.02.23.21252268 (2021) doi: 10.1101/2021.02.23.21252268.
24. Faria, N. R. et al. Genomics and epidemiology of the P.1 SARS-COV-2 lineage in Manaus, Brazil. Science eabh2644 (2021) doi: 10.1126/science.abh2644.
25. Vilar, S., Cozza, G. & Moro, S. Medicinal Chemistry and the Molecular Operating Environment (MOE): Application of QSAR and Molecular Docking to Drug Discovery. Curr Top Med Chem 8, 1555-1572 (2008).
26. Pearce, N. & Lawlor, D. A. Causal inference-so much more than statistics. Int J Epidemiol 45, 1895-1903 (2016).
27. Cathcart, A. L. et al. The dual function monoclonal antibodies VIR-7831 and VIR-7832 demonstrate potent in vitro and in vivo activity against SARS-COV-2. Biorxiv 2021.03.09.434607 (2021) doi: 10.1101/2021.03.09.434607.
28. Peacock, T. P. et al. The furin cleavage site in the SARS-COV-2 spike protein is required for transmission in ferrets. Nat Microbiol 1-11 (2021) doi: 10.1038/s41564-021-00908-w.
29. Kluyver, T. et al. Jupyter Notebooks-a publishing format for reproducible computational workflows-ePrints Soton. in 20^{th} International Conference on Electronic Publishing (2016).
30. Harris, C. R. et al. Array programming with NumPy. Nature 585, 357-362 (2020).
31. Pedregosa, F. et al. Scikit-learn: Machine Learning in Python. Journal of Machine Learning Research 12, 2825-2830 (2011).
32. Seabold, S. & Perktold, J. statsmodels: Econometric and statistical modeling with python. in 9^{th} Python in Science Conference (2010).
33. Mckinney, W. Data Structures For Statistical Computing in Python. in Proceedings of the 9^{th} Python Science Conference (eds. Walt, S. van der & Millman, J.) 56-61 (2010). doi: 10.25080/majora-92bf1922-00a.
34. Slater, G. S. C. & Birney, E. Automated generation of heuristics for biological sequence comparison. Bmc Bioinformatics 6, 31 (2005).
35. Katoh, K. & Standley, D. M. MAFFT Multiple Sequence Alignment Software Version 7: Improvements in Performance and Usability. Mol Biol Evol 30, 772-780 (2013).
36. Tarke, A. et al. Comprehensive analysis of T cell immunodominance and immunoprevalence of SARS-COV-2 epitopes in COVID-19 cases. Cell Reports Medicine 2, 100204 (2021).
37. Greaney, A. J. et al. Comprehensive mapping of mutations in the SARS-COV-2 receptor-binding domain that affect recognition by polyclonal human plasma antibodies. Cell Host Microbe 29, 463-476.e6 (2021).
38. Manfredonia, I. et al. Genome-wide mapping of therapeutically-relevant SARS-COV-2 RNA structures. Biorxiv 2020.06.15.151647 (2020) doi: 10.1101/2020.06.15.151647.
39. Murrell, B. et al. Detecting Individual Sites Subject to Episodic Diversifying Selection. Plos

Genet 8, e1002764 (2012).

### Tables

TABLE 1
Summary of analytical features. A total of 48 parameters for 14 variables were created for
5 feature groups. These capture evolutionary, immune, epidemiologic, transmissibility, and
language model predictors of spread. A detailed description of all parameters is in Table 7.

| | | | | Number |
|---|---|---|---|---|
| Feature | | | Source or | of |
| group | Variable | Meaning | reference | parameters |
| Evolution | Positive selection (FEL, | Parameters from Fixed Effects | HyPhy²² | 11 |
| | MEME) | Likelihood (FEL) and Mixed | | |
| | | Effects Model of Evolution | | |
| | | (MEME) | | |
| | Codon-SHAPE | RNA SHAPE constraint | Manfredonia et | 3 |
| | | | al. 2020³⁸ | |
| | Viral entropy | Shannon entropy at each codon | This work | 3 |
| | | position for an amino acid site | | |
| Immune | CD8 epitope escape | The frequency of SARS CoV-2 | Agerer et al. | 1 |
| | | mutations in cytotoxic | 2021¹⁶ | |
| | | lymphocyte (CTL) epitopes | | |
| | CD8 response | The percent and average CD8+ | Tarke et al. | 2 |
| | | T-cell response to an epitope in | 2021³⁶ | |
| | | patients | | |
| | CD4 response | The percent and average CD4+ | Tarke et al. | 2 |
| | | T-cell response to an epitope in | 2021³⁶ | |
| | | patients | | |
| | Antibody binding score | The estimated percent | This work | 17 |
| | | contribution of a site to binding | | |
| | | of the indicated antibody, as | | |
| | | estimated by Molecular | | |
| | | Operating Environment (MOE) | | |
| | Maximum escape | The maximum escape fraction | Greaney et al. | 1 |
| | fraction in vitro | across all conditions for that | 2021²⁰ | |
| | | mutation | | |
| Epidemiology | Variant frequency | The percent of sequences with | Calculated | 1 |
| | | the mutation | from GISAID² | |
| | Fraction of unique | The fraction of unique Spike | Calculated | 1 |
| | haplotypes | haplotypes in which a mutation is | from GISAID² | |
| | | observed | | |
| | Number of countries | The number of countries where it | Calculated | 1 |
| | | has been observed. | from GISAID² | |
| | Epi Score | The exponentially weighted | Calculated | 1 |
| | | mean rank across the other | from GISAID² | |
| | | epidemiology variables | | |
| Transmissibility | RBD expression change | Change in RBD expression due | Starr et al. | 1 |
| | | to the mutation | 2020¹⁴ | |
| | ACE2 binding change | The change in binding affinity | Starr et al. | 1 |
| | | for ACE2 | 2020¹⁴ | |
| Language | Language model | Grammaticality and semantic | Hie et al. | 2 |
| model | | change of a mutation | 2021¹⁸ | |

TABLE 2
Selected forecasted mutations. Included are forecasted mutations that are
not associated to CDC variants of concern and score above 9.7 in Epi score.

| | | | | Most | Number | |
|---|---|---|---|---|---|---|
| | Epi | | Date first | prevalent | of | |
| Mutation | Score | Spike region | reported | lineage | lineages | Counts (n) on VOCs |
| S12F | 9.8 | Signal peptide + | 2020 March | B.1.1.7 | 122 | B.1.1.7 (761), B.1.351 (9), |
| | | NTD | | | | B.1.427 (3), P.1 (70) |
| Q52R | 9.8 | Signal peptide + | 2020 May | B.1.525 | 27 | B.1.1.7 (50), B.1.427 (1) |
| | | NTD | | | | |
| S98F | 9.9 | Signal peptide + | 2020 January | B.1.221 | 166 | B.1.1.7 (4875), B.1.351 (57), |
| | | NTD | | | | B.1.427 (14) |
| D138H | 9.8 | Signal peptide + | 2020 February | B.1.1.7 | 60 | B.1.1.7 (2765), B.1.427 (1) |
| | | NTD | | | | |
| L141- | 9.8 | Signal peptide + | 2020 February | A.2.5 | 153 | B.1.1.7 (50), B.1.351 (16), |
| | | NTD | | | | B.1.427 (2), P.1 (16) |
| G142- | 9.8 | Signal peptide + | 2020 February | B.1.1.7 | 158 | B.1.1.7 (1070), B.1.351 (16), |
| | | NTD | | | | B.1.427 (2), P.1 (17) |
| M153T | 9.8 | Signal peptide + | 2020 January | B.1.1.284 | 77 | B.1.1.7 (307), B.1.427 (1) |
| | | NTD | | | | |
| L189F | 9.8 | Signal peptide + | 2020 May | B.1.258.17 | 36 | B.1.1.7 (20), P.1 (1) |
| | | NTD | | | | |
| A222V | 10 | Signal peptide + | 2020 February | B.1.177 | 284 | B.1.1.7 (449), B.1.351 (22), |
| | | NTD | | | | B.1.427 (45), P.1 (2) |
| A262S | 9.8 | Signal peptide + | 2020 February | B.1.177 | 98 | B.1.1.7 (91), B.1.351 (4), |
| | | NTD | | | | B.1.427 (1), P.1 (11) |
| S477N | 9.9 | RBM | 2020 January | B.1.160 | 145 | B.1.1.7 (22), B.1.427 (1), |
| | | | | | | P.1 (1) |
| T478K | 9.9 | RBM | 2020 April | B.1.1.519 | 38 | B.1.1.7 (3), B.1.351 (1), |
| | | | | | | B.1.427 (1) |
| S494P | 9.8 | RBM | 2020 March | B.1.575 | 112 | B.1.1.7 (1184), B.1.427 (27) |
| Q675H | 9.9 | Unannotated 2 | 2020 February | B.1.1.214 | 178 | B.1.1.7 (486), B.1.351 (40), |
| | | | | | | B.1.427 (2), P.1 (4) |
| Q677H | 9.9 | Unannotated 2 | 2020 January | B.1.2 | 240 | B.1.1.7 (2048), B.1.351 (26), |
| | | | | | | B.1.427 (51), P.1 (8) |
| T732A | 9.8 | Unannotated 2 | 2020 April | B.1.1.519 | 31 | B.1.1.7 (4), B.1.351 (1) |
| G769V | 9.8 | Unannotated 2 | 2020 March | R.1 | 154 | B.1.1.7 (53), B.1.351 (16) |
| A845S | 9.8 | Fusion | 2020 March | B.1.1.317 | 132 | B.1.1.7 (306), B.1.351 (102), |
| | | Peptides | | | | B.1.427 (1), P.1 (24) |
| F888L | 9.8 | Unannotated 3 | 2020 March | B.1.525 | 20 | B.1.1.7 (5) |
| S939F | 9.8 | Heptad | 2020 February | B.1.1.7 | 174 | B.1.1.7 (1140), B.1.351 (12), |
| | | repeat 1 | | | | B.1.427 (1), P.1 (2) |
| K1191N | 9.8 | Heptad | 2020 March | B.1.1.7 | 94 | B.1.1.7 (8097), B.1.351 (3), |
| | | repeat 2 | | | | B.1.427 (59), P.1 (2) |
| V1228L | 9.8 | Unannotated 5 | 2020 January | B.1.596 | 133 | B.1.1.7 (443), B.1.351 (2), |
| | | | | | | B.1.427 (9), P.1 (6) |

RBM = Receptor binding motif; NTD = N-terminal domain.

TABLE 3
Forecasted mutations for therapeutic antibodies. The unfiltered forecasted mutations
(including VOC mutations) were intersected with the binding epitopes of all therapeutic
antibodies for which data existed. Mutations were included if they intersected with
sites contributing at least 1% of the total binding energy of a given antibody, as
estimated by Molecular Operating Environment (MOE) program. Mutations present at less
than 1% global frequency in the most recent three months are presented in bold.

| Clinical therapeutic antibody | Forecasted mutations in epitopes |
|---|---|
| S309 | None |
| LY-CoV016 | K417N, K417T |
| REGN10987 | N439K, N440K, G446V |
| BD-368-2 | L452R, L452Q, E484K, E484Q, F490S |
| LY-CoV555 | L452R, L452Q, E484K, E484Q, F490S, S494P |
| REGN10933 | K417N, K417T, S477N, T478K, E484K, E484Q, F490S |
| CT-P59 | K417N, K417T, L452R, L452Q, E484K, E484Q, F490S, S494P |

TABLE 4
When mutations in VOCs would have been predicted to spread. This table summarizes
FIG. 7B and is subset to CDC VOC variants. The “Date first forecast to spread”
corresponds to the transition between dotted and solid lines in that graph.
“Difference (months)” is the difference in months between when a mutation
was first forecast to spread and when it reached greater than 1% prevalence.
Grey numbers in “Frequency at first forecast” indicate that these numbers
were omitted from the mean calculation, as described in the main text.

| | Date first | | | |
|---|---|---|---|---|
| | forecast to | | Difference | Frequency at first |
| Mutation | spread | Date >1% prevalence | (months) | forecast |
| S13I | 2020-07 | 2021-01 | 6 | 0.0005 |
| H69- | 2020-02 | 2020-09 | 7 | 0.0024 |
| V70- | 2020-02 | 2020-09 | 7 | 0.0024 |
| Y144- | 2020-02 | 2020-11 | 9 | 0.0024 |
| W152C | 2020-12 | 2021-01 | 1 | 0.0069 |
| K417N | 2020-10 | 2021-02 | 4 | 0.0004 |
| K417T | 2021-01 | N/A | 4 | 0.0010 |
| L452R | 2020-07 | 2021-01 | 6 | 0.0003 |
| E484K | 2020-06 | 2021-01 | 7 | 0.0001 |
| S494P | 2020-07 | N/A | 10 | 0.0002 |
| N501Y | 2020-09 | 2020-11 | 2 | 0.0004 |
| A570D | 2020-11 | 2020-11 | 0 | 0.0138 |
| D614G | 2020-02 | 2020-02 | 0 | 0.1609 |
| P681H | 2020-02 | 2020-11 | 9 | 0.0019 |
| | | Average | 5.14 | 0.0016 |

TABLE 5
When B.1.617 mutations would have been predicted to spread.
This table is the same format as Table 4, but with Spike mutations
from the B.1.617 lineage. The “Date first forecast to spread”
corresponds to the transition between dotted and solid lines
in that graph. “Difference (months)” is the difference
in months between when a mutation was first forecast to spread
and when it reached greater than 1% prevalence.

| | Date first | | | |
|---|---|---|---|---|
| | forecast to | Date >1% | | Frequency at first |
| Variant | spread | prevalence | Difference (months) | forecast |
| L452R | 2020-07 | 2021-01 | 6 | 0.000273 |
| E484Q | 2021-03 | N/A | 2 | 0.000886 |
| P681R | 2020-10 | N/A | 7 | 0.00034 |

TABLE 6
The AUROCs for each variable, and the best performing epidemiological
variable. Performance (AUROC) is measured both within the RBD and across
the whole spike protein. P- values represent difference from the best
performing epidemiological (Epi Score) variable in bold. Variables above
the bolded row indicate nominal complementarity within the RBD.

| | Spike | | RBD | |
|---|---|---|---|---|
| | AUROC | Spike p-value | AUROC | RBD p-value |
| Tarke_CD8_FreqResponse | 0.949 | 0.737 | 0.961 | 0.292 |
| codon-1-shape | 0.951 | 0.42 | 0.957 | 0.318 |
| codon-2-shape | 0.951 | 0.429 | 0.956 | 0.334 |
| log_prob_grammaticality | 0.95 | 0.898 | 0.954 | 0.367 |
| codon-0-shape | 0.951 | 0.347 | 0.954 | 0.342 |
| codon-2-entropy | 0.947 | 0.099 | 0.951 | 0.412 |
| Tarke_CD8_AvgResponse | 0.948 | 0.17 | 0.947 | 0.263 |
| FEL_a | 0.948 | 0.459 | 0.947 | 0.345 |
| MEME_a | 0.948 | 0.394 | 0.946 | 0.359 |
| EpitopeScore_S2H13 | 0.95 | 0.755 | 0.946 | 0.31 |
| EpitopeScore_S2E12 | 0.95 | 0.848 | 0.945 | 0.282 |
| EpitopeScore_S2H14 | 0.95 | 0.932 | 0.945 | 0.363 |
| EpitopeScore_REGN10987 | 0.95 | 0.538 | 0.944 | 0.527 |
| EpitopeScore_S2M11 | 0.949 | 0.244 | 0.944 | 0.76 |
| EpitopeScore_S2X35 | 0.95 | 0.581 | 0.944 | 0.508 |
| MEME_b− | 0.948 | 0.289 | 0.943 | 0.774 |
| Frac_Vars | 0.95 | 0.712 | 0.943 | 0.206 |
| Agerer_CD8_Allele Frequency | 0.95 | 0.714 | 0.943 | 0.689 |
| EpitopeScore_L28 | 0.95 | 0.471 | 0.943 | 1 |
| EpitopeScore_M28 | 0.95 | 0.279 | 0.943 | 1 |
| ACE2_Binding_Epitopes | 0.95 | 1 | 0.943 | 1 |
| EpiScore | 0.95 | N/A | 0.943 | N/A |
| EpitopeScore_X333 | 0.95 | 0.832 | 0.943 | 1 |
| Frac_HaplosWherePresent | 0.95 | 0.782 | 0.943 | 0.686 |
| EpitopeScore_LY-CoV555 | 0.949 | 0.226 | 0.943 | 0.943 |
| EpitopeScore_Brii-198 | 0.95 | 0.337 | 0.942 | 0.892 |
| codon-0-entropy | 0.944 | 0.011 | 0.942 | 0.856 |
| N_Countries | 0.95 | 0.233 | 0.942 | 0.279 |
| EpitopeScore_S2X259 | 0.95 | 0.685 | 0.942 | 0.657 |
| EpitopeScore_S2D106 | 0.95 | 0.231 | 0.942 | 0.699 |
| EpitopeScore_S304 | 0.95 | 0.368 | 0.942 | 0.455 |
| EpitopeScore_BD-368-2 | 0.949 | 0.21 | 0.942 | 0.613 |
| EpitopeScore_S2A4 | 0.95 | 0.509 | 0.942 | 0.368 |
| negative-selection | 0.949 | 0.01 | 0.941 | 0.273 |
| EpitopeScore_S309 | 0.95 | 0.389 | 0.941 | 0.292 |
| ACE2_BindingChange_Starr | 0.95 | 0.815 | 0.94 | 0.445 |
| codon-1-entropy | 0.945 | 0.033 | 0.938 | 0.022 |
| FEL_p | 0.946 | 0.022 | 0.936 | 0.309 |
| MEME_b+ | 0.946 | 0.044 | 0.934 | 0.256 |
| MEME_br | 0.946 | 0.029 | 0.929 | 0.314 |
| MEME_p | 0.945 | 0.001 | 0.929 | 0.267 |
| semantic_change | 0.946 | 0.078 | 0.929 | 0.245 |
| EpitopeScore_Brii-196 | 0.948 | 0.295 | 0.928 | 0.325 |
| FEL_b | 0.944 | 0.009 | 0.928 | 0.265 |
| MEME_w− | 0.945 | 0.023 | 0.927 | 0.269 |
| MEME_w+ | 0.945 | 0.023 | 0.927 | 0.269 |
| MaxEscapeFrac_Greaney | 0.947 | 0.224 | 0.926 | 0.297 |
| Tarke_CD4_FreqResponse | 0.948 | 0.307 | 0.926 | 0.277 |
| Tarke_CD4_AvgResponse | 0.948 | 0.262 | 0.926 | 0.261 |
| EpitopeScore_CT-P59 | 0.947 | 0.222 | 0.925 | 0.26 |
| EpitopeScore_REGN10933 | 0.948 | 0.285 | 0.925 | 0.318 |
| EpitopeScore_LY-CoV016 | 0.948 | 0.282 | 0.924 | 0.305 |
| EpitopeScore_Max | 0.946 | 0.098 | 0.923 | 0.266 |
| ExpressionChange_Starr | 0.947 | 0.12 | 0.919 | 0.183 |

TABLE 7
Example features and corresponding feature groups

| Variable name | Meaning | Feature group |
|---|---|---|
| negative-selection | Negative selection | Evolution |
| codon-2-shape | RNA shape constraint, codon position 3 | Evolution |
| codon-2-entropy | Entropy, codon position 3 | Evolution |
| codon-1-shape | RNA shape constraint, codon position 2 | Evolution |
| codon-1-entropy | Entropy, codon position 2 | Evolution |
| codon-0-shape | RNA shape constraint, codon position 1 | Evolution |
| codon-0-entropy | Entropy, codon position 1 | Evolution |
| FEL_a, FEL_b, FEL_p | Parameters from Fixed Effects Likelihood | Evolution |
| MEME_a, MEME_b+, | Parameters from Mixed Effects Model | Evolution |
| MEME_b−, MEME_br, | of Evolution | |
| MEME_p, MEME_w+, | | |
| MEME_w− | | |
| Agerer_CD8_Allele | The frequency of each escape mutation to | Immune |
| Frequency | recognition by CD8+ T-cells | |
| MaxEscapeFrac_Greaney | The maximum escape fraction across all | Immune |
| | conditions for that variant | |
| Tarke_CD8_FreqResponse | The percent of patient with a CD8+ T-cell | Immune |
| | response to that epitope | |
| Tarke_CD8_AvgResponse | The avg. CD8+ T-cell response strength in | Immune |
| | patients that responded | |
| Tarke_CD4_FreqResponse | The percent of patient with a CD4+ T-cell | Immune |
| | response to that epitope | |
| Tarke_CD4_AvgResponse | The avg. CD4+ T-cell response strength in | Immune |
| | patients that responded | |
| EpitopeScore_S309 | The percent contribution of a site for | Immune |
| | binding of the indicated antibody | |
| EpitopeScore_S304 | The percent contribution of a site for | Immune |
| | binding of the indicated antibody | |
| EpitopeScore_S2M11 | The percent contribution of a site for | Immune |
| | binding of the indicated antibody | |
| EpitopeScore_S2H14 | The percent contribution of a site for | Immune |
| | binding of the indicated antibody | |
| EpitopeScore_S2H13 | The percent contribution of a site for | Immune |
| | binding of the indicated antibody | |
| EpitopeScore_S2E12 | The percent contribution of a site for | Immune |
| | binding of the indicated antibody | |
| EpitopeScore_S2A4 | The percent contribution of a site for | Immune |
| | binding of the indicated antibody | |
| EpitopeScore_REGN10987 | The percent contribution of a site for | Immune |
| | binding of the indicated antibody | |
| EpitopeScore_REGN10933 | The percent contribution of a site for | Immune |
| | binding of the indicated antibody | |
| EpitopeScore_Max | Max percent contribution of a site to | Immune |
| | binding of any listed antibody | |
| EpitopeScore_S2M28 | The percent contribution of a site for | Immune |
| | binding of the indicated antibody | |
| EpitopeScore_LY-CoV555 | The percent contribution of a site for | Immune |
| | binding of the indicated antibody | |
| EpitopeScore_LY-CoV016 | The percent contribution of a site for | Immune |
| | binding of the indicated antibody | |
| EpitopeScore_S2L28 | The percent contribution of a site for | Immune |
| | binding of the indicated antibody | |
| EpitopeScore_CT-P59 | The percent contribution of a site for | Immune |
| | binding of the indicated antibody | |
| EpitopeScore_BD-368-2 | The percent contribution of a site for | Immune |
| | binding of the indicated antibody | |
| EpitopeScore_Brii-196 | The percent contribution of a site for | Immune |
| | binding of the indicated antibody | |
| N_Countries | The number of countries in which a variant | Epidemiology |
| | has been found (>1 seq) | |
| Frac_HaplosWherePresent | The fraction of unique haplotypes in which | Epidemiology |
| | the variant is found | |
| Frac_Vars | Mutation prevalence. The fraction of all | Epidemiology |
| | haplotype counts that contain this variant | |
| EpiScore | The exponentially weighted mean rank | Epidemiology |
| | across the other epidemiology variables | |
| ExpressionChange_Starr | Change in RBD expression due to the | Transmissibility |
| | variant | |
| ACE2_BindingChange_Starr | Change in ACE2 binding due to the variant | Transmissibility |
| ACE2_Binding_Epitopes | Percent contribution of that site to ACE2 | Transmissibility |
| | binding | |
| log_prob_grammaticality | The log probability (grammaticality) of a | Language |
| | variant | model |
| semantic_change | The semantic change of a variant | Language |
| | | model |

TABLE 8
Example mutations, EpiScore, and
most prevalent SARS-CoV-2 lineage

| | Epi | | Most prevalent |
|---|---|---|---|
| Mutation | Score | Spike region | lineage |
| D614G | 10.00 | Unannotated 2 | B.1.1.7 |
| N501Y | 9.99 | RBM | B.1.1.7 |
| P681H | 9.99 | Unannotated 2 | B.1.1.7 |
| H69− | 9.99 | Signal peptide + NTD | B.1.1.7 |
| V70− | 9.99 | Signal peptide + NTD | B.1.1.7 |
| T716I | 9.98 | Unannotated 2 | B.1.1.7 |
| Y144− | 9.98 | Signal peptide + NTD | B.1.1.7 |
| S982A | 9.97 | Unannotated 4 | B.1.1.7 |
| D1118H | 9.97 | Unannotated 4 | B.1.1.7 |
| A570D | 9.97 | Unannotated 2 | B.1.1.7 |
| A222V | 9.96 | Signal peptide + NTD | B.1.177 |
| E484K | 9.96 | RBM | B.1.351 |
| L18F | 9.95 | Signal peptide + NTD | B.1.177 |
| A701V | 9.94 | Unannotated 2 | B.1.351 |
| L5F | 9.94 | Signal peptide + NTD | B.1.1.7 |
| L452R | 9.94 | RBM | B.1.429 |
| T95I | 9.92 | Signal peptide + NTD | B.1.526 |
| Q677H | 9.92 | Unannotated 2 | B.1.2 |
| S477N | 9.92 | RBM | B.1.160 |
| D80A | 9.91 | Signal peptide + NTD | B.1.351 |
| N439K | 9.91 | RBM | B.1.258 |
| L242− | 9.91 | Signal peptide + NTD | B.1.351 |
| S98F | 9.91 | Signal peptide + NTD | B.1.221 |
| K417N | 9.91 | RBD\RBM 1 | B.1.351 |
| A243− | 9.90 | Signal peptide + NTD | B.1.351 |
| D215G | 9.90 | Signal peptide + NTD | B.1.351 |
| L241− | 9.89 | Signal peptide + NTD | B.1.351 |
| D138Y | 9.88 | Signal peptide + NTD | P.1 |
| H655Y | 9.88 | Unannotated 2 | P.1 |
| P26S | 9.87 | Signal peptide + NTD | P.1 |
| V1176F | 9.87 | Heptad repeat 2 | P.1 |
| S13I | 9.87 | Signal peptide + NTD | B.1.429 |
| T478K | 9.87 | RBM | B.1.1.519 |
| T1027I | 9.85 | Unannotated 4 | P.1 |
| Q675H | 9.85 | Unannotated 2 | B.1.1.214 |
| D253G | 9.85 | Signal peptide + NTD | B.1.526 |
| W152C | 9.84 | Signal peptide + NTD | B.1.429 |
| A67V | 9.83 | Signal peptide + NTD | B.1.525 |
| S494P | 9.83 | RBM | B.1.575 |
| V143− | 9.83 | Signal peptide + NTD | B.1.1.7 |
| R190S | 9.83 | Signal peptide + NTD | P.1 |
| T20N | 9.83 | Signal peptide + NTD | P.1 |
| P681R | 9.82 | Unannotated 2 | B.1.1.7 |
| G142− | 9.82 | Signal peptide + NTD | B.1.1.7 |
| K417T | 9.81 | RBD\RBM 1 | P.1 |
| T732A | 9.81 | Unannotated 2 | B.1.1.519 |
| S939F | 9.80 | Heptad repeat 1 | B.1.1.7 |
| G769V | 9.80 | Unannotated 2 | R.1 |
| M153T | 9.79 | Signal peptide + NTD | B.1.1.284 |
| A262S | 9.79 | Signal peptide + NTD | B.1.177 |
| A845S | 9.79 | Fusion Peptides | B.1.1.317 |
| D138H | 9.77 | Signal peptide + NTD | B.1.1.7 |
| K1191N | 9.77 | Heptad repeat 2 | B.1.1.7 |
| L189F | 9.77 | Signal peptide + NTD | B.1.258.17 |
| F888L | 9.77 | Unannotated 3 | B.1.525 |
| L141- | 9.77 | Signal peptide + NTD | A.2.5 |
| Q52R | 9.76 | Signal peptide + NTD | B.1.525 |
| V1228L | 9.76 | Unannotated 5 | B.1.596 |
| S12F | 9.76 | Signal peptide + NTD | B.1.1.7 |
| A1078S | 9.75 | Unannotated 4 | B.1.177 |
| T572I | 9.75 | Unannotated 2 | B.1.1.7 |
| P272L | 9.74 | Signal peptide + NTD | B.1.177 |
| L54F | 9.74 | Signal peptide + NTD | B.1.1.333 |
| H49Y | 9.74 | Signal peptide + NTD | B.1.564 |
| A688V | 9.74 | Unannotated 2 | B.1.1.7 |
| V1264L | 9.73 | Unannotated 5 | B.1.1.7 |
| L176F | 9.73 | Signal peptide + NTD | B.1.177.17 |
| V772I | 9.72 | Unannotated 2 | B.1.258.17 |
| A653V | 9.72 | Unannotated 2 | B.1.1.7 |
| E583D | 9.72 | Unannotated 2 | B.1.177.18 |
| W152L | 9.71 | Signal peptide + NTD | R.1 |
| T20I | 9.71 | Signal peptide + NTD | B.1.588 |
| A522S | 9.71 | RBD\RBM 2 | B.1.1.317 |
| N501T | 9.71 | RBM | B.1.517 |
| Q613H | 9.70 | Unannotated 2 | A.23.1 |
| S640F | 9.70 | Unannotated 2 | B.1.1.7 |
| W258L | 9.70 | Signal peptide + NTD | B.1.427 |
| A520S | 9.69 | RBD\RBM 2 | B.1.2 |
| V622F | 9.68 | Unannotated 2 | B.1.1.7 |
| G1219V | 9.68 | Unannotated 5 | B.1.1.7 |
| D80Y | 9.68 | Signal peptide + NTD | B.1.367 |
| N679K | 9.68 | Unannotated 2 | B.1.1.433 |
| T859N | 9.67 | Unannotated 3 | B.1.526.1 |
| G75V | 9.67 | Signal peptide + NTD | B.1.1.1 |
| T22I | 9.66 | Signal peptide + NTD | B.1.1.7 |
| M1237I | 9.66 | Unannotated 5 | B.1.1.7 |
| Q675R | 9.65 | Unannotated 2 | B.1.1.317 |
| M1229I | 9.65 | Unannotated 5 | B.1.1.7 |
| M153I | 9.65 | Signal peptide + NTD | B.1 |
| T859I | 9.65 | Unannotated 3 | B.1.2 |
| T76I | 9.65 | Signal peptide + NTD | B.1.1.7 |
| P812S | 9.63 | Unannotated 2 | B.1.1.7 |
| P812L | 9.63 | Unannotated 2 | B.1.234 |
| N440K | 9.63 | RBM | B.1.1.420 |
| D796Y | 9.63 | Unannotated 2 | B.1.474 |
| W152R | 9.61 | Signal peptide + NTD | B.1.1.7 |
| D1163Y | 9.61 | Heptad repeat 2 | B.1.177 |
| P1263L | 9.60 | Unannotated 5 | B.1 |
| P1162S | 9.60 | Unannotated 4 | B.1.1.7 |
| F157L | 9.60 | Signal peptide + NTD | A.23.1 |
| G1219C | 9.59 | Unannotated 5 | B.1.177.21 |
| D936Y | 9.58 | Heptad repeat 1 | B.1 |
| F490S | 9.58 | RBM | B.1.1.7 |
| A1020S | 9.58 | Unannotated 4 | B.1.177.87 |
| S221L | 9.58 | Signal peptide + NTD | B.1.1.7 |
| S254F | 9.56 | Signal peptide + NTD | B.1.1.7 |
| E484Q | 9.56 | RBM | B.1.617.1 |
| V367F | 9.56 | RBD\RBM 1 | A.23.1 |
| T29I | 9.56 | Signal peptide + NTD | B.1.1.486 |
| G181V | 9.55 | Signal peptide + NTD | B.1.1.7 |
| F157S | 9.54 | Signal peptide + NTD | B.1.526.1 |
| S255F | 9.52 | Signal peptide + NTD | C.30 |
| A27S | 9.51 | Signal peptide + NTD | B.1.1.7 |
| A879S | 9.49 | Unannotated 3 | B.1.351 |
| T1117I | 9.48 | Unannotated 4 | B.1.1.7 |
| T791I | 9.48 | Unannotated 2 | B.1.526.1 |
| Q1071L | 9.47 | Unannotated 4 | B.1.177.73 |
| Y144F | 9.47 | Signal peptide + NTD | B.1.1.7 |
| A899S | 9.46 | Unannotated 3 | B.1.351 |
| E1202Q | 9.45 | Unannotated 5 | B.1.1.316 |
| V308L | 9.45 | Unannotated 1 | B.1.1.7 |
| P1162L | 9.45 | Unannotated 4 | B.1.1.7 |
| P384L | 9.45 | RBD\RBM 1 | B.1.1.7 |
| P809S | 9.45 | Unannotated 2 | B.1.1.7 |
| S704L | 9.44 | Unannotated 2 | B.1.1.7 |
| H1101Y | 9.44 | Unannotated 4 | B.1.1.7 |
| H245Y | 9.44 | Signal peptide + NTD | B.1.1.7 |
| D950H | 9.43 | Heptad repeat 1 | B.1.526.1 |
| P26L | 9.43 | Signal peptide + NTD | B.1.1.372 |
| S256L | 9.42 | Signal peptide + NTD | B.1.177 |
| P9L | 9.42 | Signal peptide + NTD | B.1.1.7 |
| L938F | 9.42 | Heptad repeat 1 | B.1.1.7 |
| S1252F | 9.41 | Unannotated 5 | B.1.221 |
| K1073N | 9.41 | Unannotated 4 | C.30 |
| D796H | 9.41 | Unannotated 2 | B.1.1.318 |
| T19I | 9.38 | Signal peptide + NTD | B.1.1.7 |
| T307I | 9.36 | Unannotated 1 | B.1.1.7 |
| A706V | 9.36 | Unannotated 2 | B.1.1.7 |
| T547I | 9.36 | Unannotated 2 | B.1.1.7 |
| V1104L | 9.34 | Unannotated 4 | B.1.1.7 |
| D215Y | 9.34 | Signal peptide + NTD | B.1.126 |
| L822F | 9.34 | Fusion Peptides | B.1.1.7 |
| M177I | 9.33 | Signal peptide + NTD | B.1.1.7 |
| S940F | 9.33 | Heptad repeat 1 | B.1.234 |
| A684V | 9.33 | Unannotated 2 | B.1.1.7 |
| T51I | 9.32 | Signal peptide + NTD | B.1.177.62 |
| L452Q | 9.32 | RBM | B.1.1.1 |
| K558N | 9.32 | Unannotated 2 | B.1.1.7 |
| E96D | 9.32 | Signal peptide + NTD | B.1.1.7 |
| S94F | 9.32 | Signal peptide + NTD | B.1.1.7 |
| V1122L | 9.31 | Unannotated 4 | B.1.1.302 |
| L1063F | 9.31 | Unannotated 4 | B.1.362 |
| Y144V | 9.30 | Signal peptide + NTD | B.1.1.7 |
| D1118Y | 9.29 | Unannotated 4 | B.1.1.7 |
| G142D | 9.29 | Signal peptide + NTD | B.1.617.1 |
| T240I | 9.28 | Signal peptide + NTD | B.1.177.81 |
| A27V | 9.28 | Signal peptide + NTD | B.1.1.7 |
| G1124V | 9.27 | Unannotated 4 | B.1 |
| E154K | 9.27 | Signal peptide + NTD | B.1.617.1 |
| D80G | 9.27 | Signal peptide + NTD | B.1.526.1 |
| I68− | 9.27 | Signal peptide + NTD | B.1.1.7 |
| H69Y | 9.26 | Signal peptide + NTD | B.1.526.1 |
| L216F | 9.26 | Signal peptide + NTD | B.1.1.7 |
| T323I | 9.25 | RBD\RBM 1 | B.1.1.7 |
| D936N | 9.25 | Heptad repeat 1 | B.1.1.486 |
| T1273− | 9.25 | Unannotated 5 | B.1.1.7 |
| V70I | 9.24 | Signal peptide + NTD | B.1.1.7 |
| S689I | 9.24 | Unannotated 2 | B.1.1.28 |
| Y1272− | 9.24 | Unannotated 5 | B.1.1.7 |
| T678I | 9.24 | Unannotated 2 | B.1.1.7 |
| A67S | 9.23 | Signal peptide + NTD | B.1.1.7 |
| H1271− | 9.23 | Unannotated 5 | B.1.1.7 |
| A672V | 9.22 | Unannotated 2 | B.1.243 |
| T299I | 9.20 | Signal peptide + NTD | B.1.1.7 |
| A846V | 9.20 | Fusion Peptides | B.1.1.7 |
| F140− | 9.19 | Signal peptide + NTD | B.1.1.7 |
| L1270− | 9.19 | Unannotated 5 | B.1.1.7 |
| V1268− | 9.19 | Unannotated 5 | B.1.1.7 |
| S71− | 9.18 | Signal peptide + NTD | B.1.1.7 |
| K1269− | 9.18 | Unannotated 5 | B.1.1.7 |
| A771S | 9.17 | Unannotated 2 | B.1.2 |
| A1070V | 9.16 | Unannotated 4 | B.1.2 |
| A1020V | 9.16 | Unannotated 4 | B.1.1.7 |
| A892V | 9.15 | Unannotated 3 | B.1.1.7 |
| C1247F | 9.15 | Unannotated 5 | B.1.177 |
| P330S | 9.14 | RBD\RBM 1 | B.1.1.7 |
| F565L | 9.14 | Unannotated 2 | P.2 |
| Q414K | 9.14 | RBD\RBM 1 | B.1.214.2 |
| D215H | 9.14 | Signal peptide + NTD | B.1.177 |
| G1267− | 9.13 | Unannotated 5 | B.1.2 |
| I818V | 9.13 | Fusion Peptides | B.1.1.7 |
| M731I | 9.13 | Unannotated 2 | B.1.1.7 |
| G446V | 9.12 | RBM | B.1.1.7 |
| R214L | 9.12 | Signal peptide + NTD | B.1.1.7 |
| D1257− | 9.12 | Unannotated 5 | B.1.2 |
| Q1071H | 9.11 | Unannotated 4 | B.1.617.1 |
| E1262− | 9.11 | Unannotated 5 | B.1 |
| P1263− | 9.11 | Unannotated 5 | B.1 |
| K1266− | 9.11 | Unannotated 5 | B.1 |
| V1264− | 9.10 | Unannotated 5 | B.1 |

What is claimed is:

1. A method for predicting spread of a mutation of a pathogen, the method comprising:
obtaining values of features of the mutation of the pathogen;
applying a predictive model to the values of features of the mutation to predict a score indicative of a likelihood of spread of the mutation, wherein the predictive model is generated using training data derived from prior surveillance data of the pathogen corresponding to one or more previous spreads of the pathogen; and
determining whether the mutation of the pathogen will spread according to the predicted score.

2. The method of claim 1, wherein features of the mutation comprise one or more of epidemiology features, evolution features, transmissibility features, language model features, or immune features.

3. The method of claim 2, wherein epidemiology features comprise one or more of mutation frequency, the fraction of unique variant sequences that contain an amino acid mutation, the number of countries in which a mutation was been observed, or an epidemiology score representing an exponentially weighted mean ranking across mutation frequency, the fraction of unique variant sequences that contain an amino acid mutation, and the number of countries in which a mutation was been observed.

4. The method of claim 2 or 3, wherein language model features comprise one or more of grammaticality or semantic change scores.

5. The method of any one of claims 2-4, wherein transmissibility features comprise one or more of change in receptor binding domain (RBD) expression or ACE2 binding change.

6. The method of any one of claims 2-5, wherein immune features comprise one or more of frequency of a mutation in cytotoxic lymphocyte epitopes, percent or average CD8+ T-cell response to an epitope, percent or average CD4+ T-cell response to an epitope, an antibody binding score representing percent contribution of a site to binding of an antibody, or a maximum escape fraction for a mutation.

7. The method of any one of claims 2-6, wherein evolution features comprise one or more of positive selection features, Codon-SHAPE feature, or viral entropy features.

8. The method of claim 1, wherein applying the predictive model to features of the mutation comprises applying the predictive model only to epidemiology features.

9. The method of claim 8, wherein applying the predictive model comprises applying the predictive model only to an epidemiology score.

10. The method of any one of claims 1-9, wherein the predictive model exhibits an area under the receiving operating curve (AUROC) value of at least 0.90 for predicting 1 month in advance of a forecasted spread.

11. The method of any one of claims 1-9, wherein the predictive model exhibits an area under the receiving operating curve (AUROC) value of at least 0.85 for predicting 2 months in advance of a forecasted spread.

12. The method of any one of claims 1-9, wherein the predictive model exhibits an area under the receiving operating curve (AUROC) value of at least 0.80 for predicting at least 3 months in advance of a forecasted spread.

13. The method of any one of claims 1-9, wherein the predictive model exhibits an area under the receiving operating curve (AUROC) value of at least 0.60 for predicting at least 4 months in advance of a forecasted spread.

14. The method of any one of claims 1-9, wherein the predictive model exhibits an area under the receiving operating curve (AUROC) value of at least 0.70 for predicting at least 4 months in advance of a forecasted spread.

15. The method of any one of claims 1-9, wherein the predictive model exhibits an area under the receiving operating curve (AUROC) value of at least 0.80 for predicting at least 4 months in advance of a forecasted spread.

16. The method of any one of claims 1-9, wherein the predictive model exhibits an area under the receiving operating curve (AUROC) value of at least 0.85 for predicting at least 4 months in advance of a forecasted spread.

17. The method of any one of claims 1-9, wherein the predictive model exhibits an area under the receiving operating curve (AUROC) value of at least 0.87 for predicting at least 4 months in advance of a forecasted spread.

18. The method of any one of claims 1-17, wherein the mutation is an amino acid mutation of a protein of the pathogen.

19. The method of any one of claims 1-17, wherein the mutation is a nucleic acid mutation corresponding to an amino acid change of a protein of the pathogen.

20. The method of claim 18 or 19, further comprising predicting impact of the mutation on therapeutic efficacy of therapeutic antibody.

21. The method of claim 20, wherein predicting impact of the mutation comprises:
mapping the mutation to a specific amino acid of a protein of the pathogen; and
determining a contribution of the mutation of the specific amino acid to a binding energy between the therapeutic antibody and the protein of the pathogen.

22. The method of any one of claims 1-21, further comprising:
subsequent to determining that the mutation of the pathogen will spread according to the predicted score, identifying a pathogen variant likely to spread, the pathogen variant comprising at least the determined mutation that will spread.

23. The method of claim 22, wherein the pathogen variant further comprises at least two, at least three, at least four, at least five, at least six, at least seven, at least eight, at least nine, at least ten, at least eleven, at least twelve, at least thirteen, at least fourteen, at least fifteen at least sixteen, at least seventeen, at least eighteen, at least nineteen, at least twenty, at least twenty five, at least thirty, at least thirty five, at least forty, at least forty five, at least fifty, at least fifty five, at least sixty, at least sixty five, at least seventy, at least seventy five, at least eighty, at least eighty five, at least ninety, at least ninety five, or at least a hundred additional mutations that are predicted to likely spread.

24. The method of claim 22 or 23, wherein the identification of the pathogen variant likely to spread is based on one or more prior variants of interest or variants of concern.

25. The method of claim 24, wherein the identification of the pathogen variant likely to spread is based on one or more prior variants of interest or variants of concern and additional one or more mutations that occur at a rate of at least a threshold percentage of a most prevalent variant in the lineage.

26. The method of claim 25, wherein the threshold percentage is at least 50%, at least 55%, at least 60%, at least 65%, at least 70%, at least 75%, at least 80%, at least 85%, at least 90%, or at least 95%.

27. A method for training a predictive model capable of forecasting one or more spreading mutations of a pathogen, the method comprising:
obtaining one or more of prior surveillance data of the pathogen;
defining spread of one or more mutations in the prior surveillance data of the pathogen;
performing a feature selection process to identify one or more features informative for predicting spread of the defined one or more mutations; and
training a predictive model using training data comprising values of the identified one or more features, the training data derived from the surveillance data of the pathogen.

28. The method of claim 27, wherein defining spread of one or more mutations comprises:
for a mutation, determining one or more fold changes in frequency of the mutation within a time window in comparison to a previous time window; and
comparing the determined one or more fold changes to a threshold fold-change value.

29. The method of claim 28, wherein each of the one or more fold changes in frequency of the mutation is calculated for a country.

30. The method of claim 28, wherein each of the one or more fold changes in frequency of the mutation is calculated for a state.

31. The method of any one of claims 27-30, wherein defining spread of one or more mutations comprises:
determining spread of a first mutation of the pathogen corresponding to a first wave; and
determining spread of a second mutation of the pathogen corresponding to a second wave.

32. The method of claim 31, wherein the first wave and the second wave occur within 1 year.

33. The method of claim 31, wherein the first wave and the second wave are separated by at least 1 year.

34. The method of any one of claims 27-33, wherein the one or more features informative for predicting spread of the defined one or more mutations comprise one or more of epidemiology features, evolution features, transmissibility features, language model features, or immune features.

35. The method of claim 34, wherein epidemiology features comprise one or more of mutation frequency, the fraction of unique variant sequences that contain an amino acid mutation, the number of countries in which a mutation was been observed, or an epidemiology score representing an exponentially weighted mean ranking across mutation frequency, the fraction of unique variant sequences that contain an amino acid mutation, and the number of countries in which a mutation was been observed.

36. The method of claim 34 or 35, wherein language model features comprise one or more of grammaticality or semantic change scores.

37. The method of any one of claims 34-36, wherein transmissibility features comprise one or more of change in receptor binding domain (RBD) expression or ACE2 binding change.

38. The method of any one of claims 34-37, wherein immune features comprise one or more of frequency of a mutation in cytotoxic lymphocyte epitopes, percent or average CD8+ T-cell response to an epitope, percent or average CD4+ T-cell response to an epitope, an antibody binding score representing percent contribution of a site to binding of an antibody, or a maximum escape fraction for a mutation.

39. The method of any one of claims 34-38, wherein evolution features comprise one or more of positive selection features, Codon-SHAPE feature, or viral entropy features.

40. The method of any one of claims 1-39, wherein the pathogen is an epidemic or pandemic causing pathogen.

41. The method of any one of claims 1-40, wherein the pathogen is either influenza or SARS-CoV-2.

42. The method of claim 41, wherein the pathogen is SARS-COV-2 and wherein the mutation is located on a receptor binding domain (RBD) or on a Spike protein.

43. The method of any one of claims 1-42, wherein the surveillance data comprises one or more of genomic, transcriptomic, or proteomic surveillance data.

44. A non-transitory computer readable medium for predicting spread of a mutation of a pathogen, the non-transitory computer readable medium comprising instructions that, when executed by a processor, cause the processor to:
obtain features of the mutation of the pathogen;
apply a predictive model to features of the mutation to predict a score indicative of a likelihood of spread of the mutation, wherein the predictive model is generated using training data derived from prior surveillance data of the pathogen corresponding to one or more previous spreads of the pathogen; and
determine whether the mutation of the pathogen will spread according to the predicted score.

45. The non-transitory computer readable medium of claim 44, wherein features of the mutation comprise one or more of epidemiology features, evolution features, transmissibility features, language model features, or immune features.

46. The non-transitory computer readable medium of claim 45, wherein epidemiology features comprise one or more of mutation frequency, the fraction of unique variant sequences that contain an amino acid mutation, the number of countries in which a mutation was been observed, or an epidemiology score representing an exponentially weighted mean ranking across mutation frequency, the fraction of unique variant sequences that contain an amino acid mutation, and the number of countries in which a mutation was been observed.

47. The non-transitory computer readable medium of claim 45 or 46, wherein language model features comprise one or more of grammaticality or semantic change scores.

48. The non-transitory computer readable medium of any one of claims 45-47, wherein transmissibility features comprise one or more of change in receptor binding domain (RBD) expression or ACE2 binding change.

49. The non-transitory computer readable medium of any one of claims 45-48, wherein immune features comprise one or more of frequency of a mutation in cytotoxic lymphocyte epitopes, percent or average CD8+ T-cell response to an epitope, percent or average CD4+ T-cell response to an epitope, an antibody binding score representing percent contribution of a site to binding of an antibody, or a maximum escape fraction for a mutation.

50. The non-transitory computer readable medium of any one of claims 45-49, wherein evolution features comprise one or more of positive selection features, Codon-SHAPE feature, or viral entropy features.

51. The non-transitory computer readable medium of claim 44, wherein the instructions that cause the processor to apply the predictive model to features of the mutation further comprises instructions that, when executed by the processor, cause the processor to apply the predictive model only to epidemiology features.

52. The non-transitory computer readable medium of claim 51, wherein the instructions that cause the processor to apply the predictive model comprises instructions that, when executed by the processor, cause the processor to apply the predictive model only to an epidemiology score.

53. The non-transitory computer readable medium of any one of claims 44-52, wherein the predictive model exhibits an area under the receiving operating curve (AUROC) value of at least 0.90 for predicting 1 month in advance of a forecasted spread.

54. The non-transitory computer readable medium of any one of claims 44-52, wherein the predictive model exhibits an area under the receiving operating curve (AUROC) value of at least 0.85 for predicting 2 months in advance of a forecasted spread.

55. The non-transitory computer readable medium of any one of claims 44-52, wherein the predictive model exhibits an area under the receiving operating curve (AUROC) value of at least 0.80 for predicting at least 3 months in advance of a forecasted spread.

56. The non-transitory computer readable medium of any one of claims 44-52, wherein the predictive model exhibits an area under the receiving operating curve (AUROC) value of at least 0.60 for predicting at least 4 months in advance of a forecasted spread.

57. The non-transitory computer readable medium of any one of claims 44-52, wherein the predictive model exhibits an area under the receiving operating curve (AUROC) value of at least 0.70 for predicting at least 4 months in advance of a forecasted spread.

58. The non-transitory computer readable medium of any one of claims 44-52, wherein the predictive model exhibits an area under the receiving operating curve (AUROC) value of at least 0.80 for predicting at least 4 months in advance of a forecasted spread.

59. The non-transitory computer readable medium of any one of claims 44-52, wherein the predictive model exhibits an area under the receiving operating curve (AUROC) value of at least 0.85 for predicting at least 4 months in advance of a forecasted spread.

60. The non-transitory computer readable medium of any one of claims 44-52, wherein the predictive model exhibits an area under the receiving operating curve (AUROC) value of at least 0.87 for predicting at least 4 months in advance of a forecasted spread.

61. The non-transitory computer readable medium of any one of claims 44-60, wherein the mutation is an amino acid mutation of a protein of the pathogen.

62. The non-transitory computer readable medium of any one of claims 44-60, wherein the mutation is a nucleic acid mutation corresponding to an amino acid change of a protein of the pathogen.

63. The non-transitory computer readable medium of claim 61 or 62, further comprising instructions that, when executed by the processor, cause the processor to predict impact of the mutation on therapeutic efficacy of therapeutic antibody.

64. The non-transitory computer readable medium of claim 63, wherein the instructions that cause the processor to predict impact of the mutation further comprises instructions that, when executed by the processor, cause the processor to:
map the mutation to a specific amino acid of a protein of the pathogen; and
determine a contribution of the mutation of the specific amino acid to a binding energy between the therapeutic antibody and the protein of the pathogen.

65. The non-transitory computer readable medium of any one of claims 44-64, further comprising instructions that, when executed by the processor, cause the processor to:
subsequent to the determination that the mutation of the pathogen will spread according to the predicted score, identify a pathogen variant likely to spread, the pathogen variant comprising at least the determined mutation that will spread.

66. The non-transitory computer readable medium of claim 65, wherein the pathogen variant further comprises at least two, at least three, at least four, at least five, at least six, at least seven, at least eight, at least nine, at least ten, at least eleven, at least twelve, at least thirteen, at least fourteen, at least fifteen at least sixteen, at least seventeen, at least eighteen, at least nineteen, at least twenty, at least twenty five, at least thirty, at least thirty five, at least forty, at least forty five, at least fifty, at least fifty five, at least sixty, at least sixty five, at least seventy, at least seventy five, at least eighty, at least eighty five, at least ninety, at least ninety five, or at least a hundred additional mutations that are predicted to likely spread.

67. The non-transitory computer readable medium of claim 65 or 66, wherein the identification of the pathogen variant likely to spread is based on one or more prior variants of interest or variants of concern.

68. The non-transitory computer readable medium of claim 67, wherein the identification of the pathogen variant likely to spread is based on one or more prior variants of interest or variants of concern and additional one or more mutations that occur at a rate of at least a threshold percentage of a most prevalent variant in the lineage.

69. The non-transitory computer readable medium of claim 68, wherein the threshold percentage is at least 50%, at least 55%, at least 60%, at least 65%, at least 70%, at least 75%, at least 80%, at least 85%, at least 90%, or at least 95%.

70. A non-transitory computer readable medium for training a predictive model capable of forecasting one or more spreading mutations of a pathogen, the non-transitory computer readable medium comprising instructions that, when executed by a processor, cause the processor to:
obtain one or more of prior surveillance data of the pathogen;
define spread of one or more mutations in the prior surveillance data of the pathogen;
perform a feature selection process to identify one or more features informative for predicting spread of the defined one or more mutations; and
train a predictive model using training data comprising values of the identified one or more features, the training data derived from the surveillance data of the pathogen.

71. The non-transitory computer readable medium of claim 70, wherein the instructions that cause the processor to define spread of one or more mutations further comprises instructions that, when executed by the processor, cause the processor to:
for a mutation, determine one or more fold changes in frequency of the mutation within a time window in comparison to a previous time window; and
compare the determined one or more fold changes to a threshold fold-change value.

72. The non-transitory computer readable medium of claim 71, wherein each of the one or more fold changes in frequency of the mutation is calculated for a country.

73. The non-transitory computer readable medium of claim 71, wherein each of the one or more fold changes in frequency of the mutation is calculated for a state.

74. The non-transitory computer readable medium of any one of claims 70-73, wherein the instructions that cause the processor to define spread of one or more mutations further comprises instructions that, when executed by the processor, cause the processor to:
determine spread of a first mutation of the pathogen corresponding to a first wave; and
determine spread of a second mutation of the pathogen corresponding to a second wave.

75. The non-transitory computer readable medium of claim 74, wherein the first wave and the second wave occur within 1 year.

76. The non-transitory computer readable medium of claim 74, wherein the first wave and the second wave are separated by at least 1 year.

77. The non-transitory computer readable medium of any one of claims 70-76, wherein the one or more features informative for predicting spread of the defined one or more mutations comprise one or more of epidemiology features, evolution features, transmissibility features, language model features, or immune features.

78. The non-transitory computer readable medium of claim 77, wherein epidemiology features comprise one or more of mutation frequency, the fraction of unique variant sequences that contain an amino acid mutation, the number of countries in which a mutation was been observed, or an epidemiology score representing an exponentially weighted mean ranking across mutation frequency, the fraction of unique variant sequences that contain an amino acid mutation, and the number of countries in which a mutation was been observed.

79. The non-transitory computer readable medium of claim 77 or 78, wherein language model features comprise one or more of grammaticality or semantic change scores.

80. The non-transitory computer readable medium of any one of claims 77-79, wherein transmissibility features comprise one or more of change in receptor binding domain (RBD) expression or ACE2 binding change.

81. The non-transitory computer readable medium of any one of claims 77-80, wherein immune features comprise one or more of frequency of a mutation in cytotoxic lymphocyte epitopes, percent or average CD8+ T-cell response to an epitope, percent or average CD4+ T-cell response to an epitope, an antibody binding score representing percent contribution of a site to binding of an antibody, or a maximum escape fraction for a mutation.

82. The non-transitory computer readable medium of any one of claims 77-81, wherein evolution features comprise one or more of positive selection features, Codon-SHAPE feature, or viral entropy features.

83. The non-transitory computer readable medium of any one of claims 44-82, wherein the pathogen is an epidemic or pandemic causing pathogen.

84. The non-transitory computer readable medium of any one of claims 44-83, wherein the pathogen is either influenza or SARS-COV-2.

85. The non-transitory computer readable medium of claim 84, wherein the pathogen is SARS-COV-2 and wherein the mutation is located on a receptor binding domain (RBD) or on a Spike protein.

86. The non-transitory computer readable medium of any one of claims 44-85, wherein the surveillance data comprises one or more of genomic, transcriptomic, or proteomic surveillance data.

87. A method for identifying a pathogen variant likely to spread, the method comprising:
obtaining values of features of one or more mutations of a pathogen;
for one of the one or more mutations:
applying a predictive model to values of features of the mutation to predict a score indicative of a likelihood of spread of the mutation, wherein the predictive model is generated using training data derived from prior surveillance data of the pathogen corresponding to one or more previous spreads of the pathogen: and
determining that the mutation will spread according to the predicted score; and
identifying a pathogen variant likely to spread, the pathogen variant comprising at least the determined mutation that will spread.

88. The method of claim 87, wherein the pathogen variant further comprises at least two, at least three, at least four, at least five, at least six, at least seven, at least eight, at least nine, at least ten, at least eleven, at least twelve, at least thirteen, at least fourteen, at least fifteen at least sixteen, at least seventeen, at least eighteen, at least nineteen, at least twenty, at least twenty five, at least thirty, at least thirty five, at least forty, at least forty five, at least fifty, at least fifty five, at least sixty, at least sixty five, at least seventy, at least seventy five, at least eighty, at least eighty five, at least ninety, at least ninety five, or at least a hundred additional mutations that are predicted to likely spread.

89. The method of claim 87 or 88, wherein the identification of the pathogen variant likely to spread is based on one or more prior variants of interest or variants of concern.

90. The method of claim 89, wherein the identification of the pathogen variant likely to spread is based on one or more prior variants of interest or variants of concern and additional one or more mutations that occur at a rate of at least a threshold percentage of a most prevalent variant in the lineage.

91. The method of claim 90, wherein the threshold percentage is at least 50%, at least 55%, at least 60%, at least 65%, at least 70%, at least 75%, at least 80%, at least 85%, at least 90%, or at least 95%
